SEQUENCE LISTING (24 sequences)

SEQ ID NO: 1 - RNA, 129 nt, synthetic construct
  misc_feature 1..129: note = Synthetic Construct
  source 1..129: mol_type = other RNA
ggcgaactagtattcttctggtccccacagactcagagagaacccgccaccatgagagtgatggcccccagaaccctgatcctgctgctgtctggcgccctggccctgacagagacatgggccggaagc

SEQ ID NO: 2 - RNA, 488 nt, synthetic construct
  misc_feature 1..488: note = Synthetic Construct
  source 1..488: mol_type = other RNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 3 - RNA, 51 nt, synthetic construct
  misc_feature 1..51: note = Synthetic Construct
  source 1..51: mol_type = other RNA
ggcgaactagtattcttctggtccccacagactcagagagaacccgccacc

SEQ ID NO: 4 - DNA, 51 bp, synthetic construct
  misc_feature 1..51: note = Synthetic Construct
  source 1..51: mol_type = other DNA
ggcgaactagtattcttctggtccccacagactcagagagaacccgccacc

SEQ ID NO: 5 - RNA, 39 nt, synthetic construct
  misc_feature 1..39: note = Synthetic Construct
  source 1..39: mol_type = other RNA
ttcttctggtccccacagactcagagagaacccgccacc

SEQ ID NO: 6 - DNA, 39 bp, synthetic construct
  misc_feature 1..39: note = Synthetic Construct
  source 1..39: mol_type = other DNA
ttcttctggtccccacagactcagagagaacccgccacc

SEQ ID NO: 7 - RNA, 78 nt, synthetic construct
  misc_feature 1..78: note = Synthetic Construct
  source 1..78: mol_type = other RNA
atgagagtgatggcccccagaaccctgatcctgctgctgtctggcgccctggccctgacagagacatgggccggaagc

SEQ ID NO: 8 - DNA, 78 bp, synthetic construct
  misc_feature 1..78: note = Synthetic Construct
  source 1..78: mol_type = other DNA
atgagagtgatggcccccagaaccctgatcctgctgctgtctggcgccctggccctgacagagacatgggccggaagc

SEQ ID NO: 9 - protein, 26 aa, synthetic construct
  REGION 1..26: note = Synthetic Construct
MRVMAPRTLILLLSGALALTETWAGS

SEQ ID NO: 10 - RNA, 165 nt, synthetic construct
  misc_feature 1..165: note = Synthetic Construct
  source 1..165: mol_type = other RNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 11 - DNA, 165 bp, synthetic construct
  misc_feature 1..165: note = Synthetic Construct
  source 1..165: mol_type = other DNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 12 - protein, 55 aa, synthetic construct
  REGION 1..55: note = Synthetic Construct
IVGIVAGLAVLAVVVIGAVVATVMCRRKSSGGKGGSYSQAASSDSAQGSDVSLTA

SEQ ID NO: 13 - RNA, 317 nt, synthetic construct
  misc_feature 1..317: note = Synthetic Construct
  source 1..317: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 14 - DNA, 311 bp, synthetic construct
  misc_feature 1..311: note = Synthetic Construct
  source 1..311: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 15 - RNA, 136 nt, synthetic construct
  misc_feature 1..136: note = Synthetic Construct
  source 1..136: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 16 - DNA, 136 bp, synthetic construct
  misc_feature 1..136: note = Synthetic Construct
  source 1..136: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 17 - RNA, 143 nt, synthetic construct
  misc_feature 1..143: note = Synthetic Construct
  source 1..143: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccg

SEQ ID NO: 18 - DNA, 143 bp, synthetic construct
  misc_feature 1..143: note = Synthetic Construct
  source 1..143: mol_type = other DNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccg

SEQ ID NO: 19 - RNA, 30 nt, synthetic construct
  misc_feature 1..30: note = Synthetic Construct
  source 1..30: mol_type = other RNA
ggcggctctggaggaggcggctccggaggc

SEQ ID NO: 20 - DNA, 30 bp, synthetic construct
  misc_feature 1..30: note = Synthetic Construct
  source 1..30: mol_type = other DNA
ggcggctctggaggaggcggctccggaggc

SEQ ID NO: 21 - protein, 10 aa, synthetic construct
  REGION 1..10: note = Synthetic Construct
GGSGGGGSGG

SEQ ID NO: 22 - DNA, 129 bp, synthetic construct
  misc_feature 1..129: note = Synthetic Construct
  source 1..129: mol_type = other DNA
ggcgaactagtattcttctggtccccacagactcagagagaacccgccaccatgagagtgatggcccccagaaccctgatcctgctgctgtctggcgccctggccctgacagagacatgggccggaagc

SEQ ID NO: 23 - DNA, 488 bp, synthetic construct
  misc_feature 1..488: note = Synthetic Construct
  source 1..488: mol_type = other DNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 24 - RNA, 740 nt, synthetic construct
  misc_feature 1..740: note = Synthetic Construct
  misc_feature 1..2: note = Linked by (5'->5')-pp(s)p- as shown in Table 6 and in Fig. 18 of the specification
  misc_difference 132: note = n = A,T,C,G or U
  misc_feature 132: note = Exists as polynucleotide sequence(s) encoding one or more neoepitopes, as defined in the specification (e.g., FIG. 17)
  source 1..740: mol_type = other RNA
ggggcgaactagtattcttctggtccccacagactcagagagaacccgccaccatgagagtgatggcccccagaaccctgatcctgctgctgtctggcgccctggccctgacagagacatgggccggaagcnatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2023/0390485 A1
(43) Pub. Date: Dec. 7, 2023

(54) SYSTEMS AND METHODS FOR PRODUCING PHARMACEUTICAL COMPOSITIONS USING PERISTALTIC PUMPS AND DAMPENERS

(71) Applicants: Genentech, South San Francisco, CA (US); BioNTech SE, Mainz (DE)

(72) Inventors: Robert Daniel OVADIA, Daly City, CA (US); Christopher Andrew Petry, Oakland, CA (US); Fredric John Lim, Fremont, CA (US); Heinrich Haas, Mainz (DE); Sebastian Hoerner, Mainz (DE); Ferdia Bates, Mainz (DE); Tobias Kind, Mainz (DE); Peer Andre Berger, Mainz (DE)

(21) Appl. No.: 18/180,073

(22) Filed: Mar. 7, 2023

Related U.S. Application Data
(63) Continuation of application No. PCT/US21/49261, filed on Sep. 7, 2021.
(60) Provisional application No. 63/075,723, filed on Sep. 8, 2020.

(51) Int. Cl. A61M 5/142

(52) U.S. Cl. CPC A61M 5/14228; A61M 2205/07

(57) ABSTRACT

Provided herein are methods and systems for use with peristaltic pumps. More particularly, provided herein are peristaltic pump systems that include a dampener for reducing the pulsations of the flowrate from the peristaltic pump system to produce, mix, transfer and/or manufacture pharmaceutical compositions and formulations, including pharmaceutical compositions and formulations comprising lipids and RNA.

### CROSS-REFERENCE TO RELATED APPLICATIONS

This application claims priority to U.S. Provisional Patent Application No. 63/075,723, filed Sep. 8, 2020, which is incorporated herein by reference in its entirety.

### SEQUENCE LISTING

The present application contains a Sequence Listing, The content of the following submission on ASCII text file is incorporated herein by reference in its entirety: a computer readable form (CRF) of the Sequence Listing (file name: 146392048140SEQLIST.TXT, date recorded: Sep. 3, 2021, size: 9,549 bytes).

### FIELD

This disclosure relates to methods and systems for producing, mixing, transferring and/or manufacturing pharmaceutical compositions and formulations. In some embodiments, tubing kits for use with peristaltic pumps for forming a pharmaceutical mixture are provided. More particularly, this disclosure relates to peristaltic pump systems that includes a dampener for reducing the pulsations of the flowrate from the peristaltic pump system to produce, mix, transfer and/or manufacture pharmaceutical compositions and formulations, including pharmaceutical compositions and formulations comprising lipids (e.g., liposomes or lipoplexes) and RNA.

### BACKGROUND

Peristaltic pumps are positive displacement pumps that can be used for pumping a variety of fluids. Typically, a peristaltic pump includes a circulate pump casing with a tube fitted or connected inside of the casing and a rotor that compresses the tube. The rotor includes a plurality of rollers attached to the external circumference of the rotor. As the rotor turns, the part of the tube compressed is occluded, thereby forcing the fluid to be moved through the tube. Because a fixed amount of fluid is pumped per rotation, peristaltic pumps can be used to roughly measure the amount of fluid to be pumped.

One major drawback of peristaltic pumps is that they can provide uneven flow. Due to the use of rollers to compress the tubing inside the pump casing, the flow rate from a peristaltic pump pulses or oscillates. Accordingly, peristaltic pumps are less suitable where a smooth consistent flow is required.

Nucleic acids like DNA and RNA are of increasing interest for various therapeutic treatments. Various reports describe approaches for administration of nucleic acids. See, e.g., U.S. Pat. No. 10,485,884, herein incorporated by reference for all purposes. One approach is leveraging the ability of cationic liposomes (which induce DNA/RNA condensations) to facilitate cellular uptake of DNA or RNA into specific cells. The cationic liposomes usually consist of a cationic lipid, like DOTMA and/or DOTAP, and one or more helper lipids, like DOPE. So-called lipoplexes' can be formed from the cationic (positively charged) liposomes and the anionic (negatively charged) nucleic acid. Lipoplexes may form spontaneously by mixing the nucleic acid with the liposomes, driven by electrostatic interactions between the positively charged liposomes and the negatively charged nucleic acid. Accordingly, methods and systems for producing, mixing, transferring and/or manufacturing pharmaceutical compositions and formulations comprising RNA and lipid (e.g., liposomes) are required.

### SUMMARY

Provided herein are peristaltic pump systems that include a dampener for reducing the pulsations or oscillations of the flowrate from the peristaltic pumps in the system. In some embodiments, these systems are useful to produce, mix, transfer and/or manufacture pharmaceutical compositions and formulations, including, e.g., compositions and formulations comprising RNA and lipids, including lipoplexes or liposomes. As explained above, the flowrate from a peristaltic pump can pulse or oscillate over time due to the nature of peristaltic pumps. Thus, peristaltic pumps may not be suitable for certain uses where a smooth or consistent flow rate is required. An example of such a use can be when the peristaltic pumps are used to displace pharmaceutical compositions. These pharmaceutical compositions can include delicate and expensive components. In addition, the amount of a given component in the pharmaceutical composition can be critical to whether that pharmaceutical composition will be effective and safe for its intended use. For example, pharmaceutical compositions and formulations comprising RNA and lipids (such as lipoplexes or liposomes) are sensitive to: (1) the dynamic ratio of nucleic acids and lipids/liposomes when they are formed upon mixing and; (2) the average flow rate used during mixing. If the dynamic flow rate of the nucleic acids varies dynamically during operation, the ratio of nucleic acids to lipids/liposomes will vary throughout the mixing operation resulting in greater heterogeneity in the quality attributes of the resulting lipoplexes (size, polydispersity index, surface charge, etc.). Syringe pumps can be used to mix nucleic acids and liposomes/lipids (including, e.g., RNA and lipids) to form lipoplexes useful in manufacturing RNA vaccines (see, e.g., Oberli M. A et al. Nano Lett. 2017, 17, 1326-1335, or Kauffman, K. J. et al. Nano Lett. 2015, 15, 7300-7306; see also WO2019077053). Syringe pumps generate flow that has relatively low pulsation and the mixing ratio of two or more solutions can be well-controlled. However, normal syringes do not offer a closed, aseptic boundary outside of a Grade A cleanroom environment because the inside of the syringe barrel is exposed to the ambient environment before the syringe plunger is pulled to load fluid into the syringe barrel. This is particularly critical for systems in which the liposomes and/or lipoplex are too big to pass through a sterilizing grade filter and cannot be terminally sterilized without significant degradation. Peristaltic pumps, by contrast, can be used with fully closed fluid paths that do not require operation in a Grade A cleanroom environment to maintain aseptic processing and sterility assurance. This is a large advantage, because Grade A cleanroom environments are costly to maintain and require time-consuming environmental monitoring controls. Accordingly, Applicants have discovered methods and systems using peristaltic pumps that includes a dampener to drastically reduce the pulsations or oscillations from peristaltic pumps useful to produce, mix, transfer, and/or manufacture pharmaceutical compositions and formulations, including, e.g., compositions and formulations comprising RNA and lipids, including lipoplexes or liposomes, e.g., RNA vaccines. Although the dampeners disclosed herein are discussed in combination with peristaltic pumps, the pump system does not necessarily have to be a peristaltic pump system, as dampeners could be combined with any pumping system that generates pulses as part of its mechanism of action (including, e.g., membrane, piston, etc.). For example, a syringe pump system can use the dampeners disclosed herein. In some embodiments, these methods and systems using peristaltic pumps that includes a dampener are suitable to ensure smooth or consistent flow rate to produce, mix, transfer and/or manufacture pharmaceutical compositions comprising RNA and lipids (including lipoplexes or liposomes), including, e.g., RNA vaccines.

In some embodiments, a tubing kit for forming a mixture includes: a first portion of tubing configured to be fluidly connected to a container containing a first composition; a second portion of tubing configured to be fluidly connected to a container containing a second composition; a dampener fluidly connected to the first portion of tubing and fluidly connected to the second portion of tubing; a mixer for mixing the first composition from the first portion of tubing and the second composition from the second portion of tubing; a mixture container for collecting the mixed first composition and second composition from the mixer, wherein the first portion of tubing is configured to be connected to at least one peristaltic pump head for pumping the first composition from the container containing the first composition to the mixture container, and the second portion of tubing is configured to be connected to at least one peristaltic pump head for pumping the second composition from the container containing the second composition to the mixture container. In some embodiments, the dampener comprises an enclosed volume of fluid. In some embodiments, the fluid is air. In some embodiments, the dampener is a tubing dampener. In some embodiments, the dampener comprises a flexible membrane. In some embodiments, the tubing kit includes a first tee connector that fluidly connects the dampener, the first portion of tubing, and a first mixer input portion of tubing, wherein the first mixer input portion of tubing fluidly connects to the mixer. In some embodiments, the tubing kit includes a second tee connector that fluidly connects the dampener, the second portion of tubing, and a second mixer input portion of tubing, wherein the second mixer input portion of tubing fluidly connects to the mixer. In some embodiments, the first portion of tubing comprises a first segment of tubing and a second segment of tubing, wherein the first segment of tubing and the second segment of tubing are fluidly connected in parallel. In some embodiments, the first segment of tubing is configured to be connected to a first peristaltic pump head, and the second segment of tubing is configured to be connected to a second peristaltic pump head. In some embodiments, the second portion of tubing comprises a third segment of tubing and a fourth segment of tubing, wherein the third portion of tubing and the fourth portion of tubing are fluidly connected in parallel. In some embodiments, the third segment of tubing is configured to be connected to a third peristaltic pump head, and the fourth segment of tubing is configured to be connected to a fourth peristaltic pump head. In some embodiments, the mixer comprises an input fluidly connected to the first portion of tubing, an input fluidly connected to the second portion of tubing, and output fluidly connected to the mixture container. In some embodiments, the mixer comprises a Y-connector, a helical mixer, or a static mixer. In some embodiments, the tubing kit includes a first dampener connector that fluidly connects the first portion of tubing to the dampener and to the mixer and a second dampener connector that fluidly connects the second portion of the tubing to the dampener and to the mixer. In some embodiments, the mixture container is a bag, vessel, or bottle.

In some embodiments, a system for forming a pharmaceutical composition or mixture of pharmaceutical compositions, the system includes: a first container containing a first pharmaceutical composition; a second container containing a second pharmaceutical composition; a first portion of tubing fluidly connected to the first container; a second portion of tubing fluidly connected to the second container; a dampener fluidly connected to the first portion of tubing and fluidly connected to the second portion of tubing; a mixer for mixing the first pharmaceutical composition from the first portion of tubing and the second pharmaceutical composition from the second portion of tubing; and a mixture container for collecting the mixed first pharmaceutical composition and second pharmaceutical composition from the mixer. In some embodiments, the system includes at least one peristaltic pump head connected to the first portion of tubing for pumping the first composition from the container containing the first composition to the mixture container, and at least one peristaltic pump connected to the second portion of tubing for pumping the second composition from the container containing the first composition to the mixture container. In some embodiments, the first composition or second composition comprises a nucleic acid, one or more lipids, one or more proteins, or a buffer. In some embodiments, the first composition comprises a nucleic acid and the second composition comprises one or more lipids. In some embodiments, the first composition comprises RNA and the second composition comprises one or more lipids. In some embodiments, the RNA comprises one or more polynucleotides encoding 10-20 neoepitopes resulting from cancer-specific somatic mutations present in a tumor specimen. In some embodiments, the RNA is formulated in a lipoplex nanoparticle or liposome. In some embodiments, the lipoplex nanoparticle or liposome comprises one or more lipids that form a multilamellar structure that encapsulates the RNA. In some embodiments, the one or more lipids comprises at least one cationic lipid and at least one helper lipid. In some embodiments, the one or more lipids comprises (R)-N,N,N-trimethyl-2,3-dioleyloxy-1-propanaminium chloride (DOTMA) and 1,2-dioleoyl-sn-glycero-3-phosphoethanolamine (DOPE). In some embodiments, at physiological pH the overall charge ratio of positive charges to negative charges of the liposome is 1.3:2 (0.65). In some embodiments, the RNA comprises an RNA molecule comprising, in the 5′→3′ direction: (1) a 5′ cap; (2) a 5′ untranslated region (UTR); (3) a polynucleotide sequence encoding a secretory signal peptide; (4) a polynucleotide sequence encoding the one or more neoepitopes resulting from cancer-specific somatic mutations present in a tumor specimen; (5) a polynucleotide sequence encoding at least a portion of a transmembrane and cytoplasmic domain of a major histocompatibility complex (MHC) molecule; (6) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (7) a poly(A) sequence. In some embodiments, the RNA molecule further comprises a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction. In some embodiments, the amino acid linker comprises the sequence GGSGGGGSGG (SEQ ID NO:21). In some embodiments, the polynucleotide sequence encoding the amino acid linker comprises the sequence GGCGGCUCUGGAGGAGGCGGCUCCGGAGGC (SEQ ID NO:19). In some embodiments, the RNA molecule further comprises, in the 5′→3′ direction: at least a second linker-epitope module, wherein the at least second linker-epitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-epitope module is different from the neoepitope of the second linker-epitope module. In some embodiments, the RNA molecule comprises 5 linker-epitope modules, and wherein the 5 linker-epitope modules each encode a different neoepitope. In some embodiments, the RNA molecule comprises 10 linker-epitope modules, and wherein the 10 linker-epitope modules each encode a different neoepitope. In some embodiments, the RNA molecule comprises 20 linker-epitope modules, and wherein the 20 linker-epitope modules each encode a different neoepitope. In some embodiments, the RNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule. In some embodiments, the 5′ cap comprises a D1 diastereoisomer of the structure:

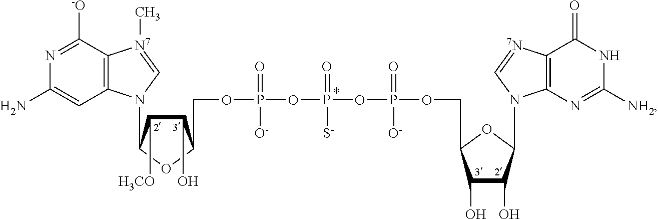

In some embodiments, the 5′ UTR comprises the sequence UUCUUCUGGUCCCCACAGACUCAGAGAGAACCCGCCACC (SEQ ID NO:5). In some embodiments, the 5′ UTR comprises the sequence

GGCGAACUAGUAUUCUUCUGGUCCCCACAGACUCAGAGAG
AACCCGCCACC

In some embodiments, the secretory signal peptide comprises the amino acid sequence MRVMAPRTLILLLSGALALTETWAGS (SEQ ID NO:9). In some embodiments, the polynucleotide sequence encoding the secretory signal peptide comprises the sequence
AUGAGAGUGAUGGCCCCCAGAACCCUGAUCCUGCUGCUGUCUGGCGCCCUGGC CCUGACAGAGACAUGGGCCGGAAGC (SEQ ID NO:7). In some embodiments, the at least portion of the transmembrane and cytoplasmic domain of the MHC molecule comprises the amino acid sequence

IVGIVAGLAVLAVVVIGAVVATVMCRRKSSGGKGGSYSQA
ASSDSAQGSDVSLTA

In some embodiments, the polynucleotide sequence encoding the at least portion of the transmembrane and cytoplasmic domain of the MHC molecule comprises the sequence
AUCGUGGGAAUUGUGGCAGGACUGGCAGUGCUGGCCGUGGUGGUGAUCGGAG CCGUGGUGGCUACCGUGAUGUGCAGACGGAAGUCCAGCGGAGGCAAGGGCGGC AGCUACAGCCAGGCCGCCAGCUCUGAUAGCGCCCAGGGCAGCGACGUGUCACU GACAGCC (SEQ ID NO:10). In some embodiments, the 3′ untranslated region of the AES mRNA comprises the sequence
CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACU CACCACCUCUGCUAGUUCCAGACACCUCC (SEQ ID NO:15). In some embodiments, the non-coding RNA of the mitochondrially encoded 12S RNA comprises the sequence CAAGCACGCAGCAAUGCAGCUCAAAACGCUUAGCCUAGCCACACCCCCACGGG AAACAGCAGUGAUUAACCUUUAGCAAUAAACGAAAGUUUAACUAAGCUAUAC UAACCCCAGGGUUGGUCAAUUUCGUGCCAGCCACACCG (SEQ ID NO:17). In some embodiments, the 3′ UTR comprises the sequence

CUCGAGCUGGUACUGCAUGCACGCAAUGCUAGCUGCCCCU
UUCCCGUCCUGGGUACCCCGAGUCUCCCCCGACCUCGGGU
CCCAGGUAUGCUCCCACCUCCACCUGCCCCACUCACCACC
UCUGCUAGUUCCAGACACCUCCCAAGCACGCAGCAAUGCA
GCUCAAAACGCUUAGCCUAGCCACACCCCCACGGGAAACA
GCAGUGAUUAACCUUUAGCAAUAAACGAAAGUUUAACUAA
GCUAUACUAACCCCAGGGUUGGUCAAUUUCGUGCCAGCCA
CACCGAGACCUGGUCCAGAGUCGCUAGCCGCGUCGCU

In some embodiments, the poly(A) sequence comprises 120 adenine nucleotides. In some embodiments, the RNA comprises an RNA molecule comprising, in the 5′→3′ direction: the polynucleotide sequence
GGCGAACUAGUAUUCUUCUGGUCCCCACAGACUCAGAGAGAACCCGCCACCAU GAGAGUGAUGGCCCCCAGAACCCUGAUCCUGCUGCUGUCUGGCGCCCUGGCCC UGACAGAGACAUGGGCCGGAAGC (SEQ ID NO:1); a polynucleotide sequence encoding the one or more neoepitopes resulting from cancer-specific somatic mutations present in the tumor specimen; and the polynucleotide sequence

(SEQ ID NO: 2)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCCUAGUAACUCGAGCUGGUACUGCAUGCACGCA
AUGCUAGCUGCCCCUUUCCCGUCCUGGGUACCCCGAGUCUCCCCCGACC
UCGGGUCCCAGGUAUGCUCCCACCUCCACCUGCCCCACUCACCACCUCU
GCUAGUUCCAGACACCUCCCAAGCACGCAGCAAUGCAGCUCAAAACGCU
UAGCCUAGCCACACCCCCACGGGAAACAGCAGUGAUUAACCUUUAGCAA
UAAACGAAAGUUUAACUAAGCUAUACUAACCCCAGGGUUGGUCAAUUUC
GUGCCAGCCACACCGAGACCUGGUCCAGAGUCGCUAGCCGCGUCGCU.

In some embodiments, a method for transferring pharmaceutical composition using peristaltic pumps includes: pumping a first composition from a first container through a first portion of tubing using at least one peristaltic pump; pumping a second composition from a second container through a second portion of tubing using at least one peristaltic pump; and dampening pulse in a fluid flow of the first composition in the first portion of tubing and dampening pulses in a fluid flow of the second composition in the second portion of tubing using a dampener fluid connected to the first portion of tubing and fluidly connected to the second portion of tubing. In some embodiments, the method includes mixing the first composition from the first portion of tubing and the second composition from the second portion of tubing in a mixer fluidly connected to the first portion of tubing and the second portion of tubing. In some embodiments, the method includes depositing the mixture containing the first composition and the second composition into a mixture container that is fluidly connected to the mixture. In some embodiments, the first composition or second composition comprises a nucleic acid, one or more lipids, one or more proteins, or a buffer. In some embodiments, the first composition comprises a nucleic acid and the second composition comprises one or more lipids. In some embodiments, the first composition comprises RNA and the second composition comprises one or more lipids. In some embodiments, the RNA comprises one or more polynucleotides encoding 10-20 neoepitopes resulting from cancer-specific somatic mutations present in a tumor specimen. In some embodiments, the RNA is formulated in a lipoplex nanoparticle or liposome. In some embodiments, the lipoplex nanoparticle or liposome comprises one or more lipids that form a multilamellar structure that encapsulates the RNA. In some embodiments, the one or more lipids comprises at least one cationic lipid and at least one helper lipid. In some embodiments, the one or more lipids comprises (R)-N,N,N-trimethyl-2,3-dioleyloxy-1-propanaminium chloride (DOTMA) and 1,2-dioleoyl-sn-glycero-3-phosphoethanolamine (DOPE). In some embodiments, at physiological pH the overall charge ratio of positive charges to negative charges of the liposome is 1.3:2 (0.65). In some embodiments, the RNA comprises an RNA molecule comprising, in the 5′→3′ direction: (1) a 5′ cap; (2) a 5′ untranslated region (UTR); (3) a polynucleotide sequence encoding a secretory signal peptide; (4) a polynucleotide sequence encoding the one or more neoepitopes resulting from cancer-specific somatic mutations present in a tumor specimen; (5) a polynucleotide sequence encoding at least a portion of a transmembrane and cytoplasmic domain of a major histocompatibility complex (MHC) molecule; (6) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (7) a poly(A) sequence. In some embodiments, the RNA molecule further comprises a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction. In some embodiments, the amino acid linker comprises the sequence GGSGGGGSGG (SEQ ID NO:21). In some embodiments, the polynucleotide sequence encoding the amino acid linker comprises the sequence GGCGGCUCUGGAGGAGGCGGCUCCGGAGGC (SEQ ID NO:19). In some embodiments, the RNA molecule further comprises, in the 5′→3′ direction: at least a second linker-epitope module, wherein the at least second linker-epitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′ 43′ direction; and wherein the neoepitope of the first linker-epitope module is different from the neoepitope of the second linker-epitope module. In some embodiments, the RNA molecule comprises 5 linker-epitope modules, and wherein the 5 linker-epitope modules each encode a different neoepitope. In some embodiments, the RNA molecule comprises 10 linker-epitope modules, and wherein the 10 linker-epitope modules each encode a different neoepitope. In some embodiments, the RNA molecule comprises 20 linker-epitope modules, and wherein the 20 linker-epitope modules each encode a different neoepitope. In some embodiments, the RNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule. In some embodiments, the 5′ cap comprises a D1 diastereoisomer of the structure:

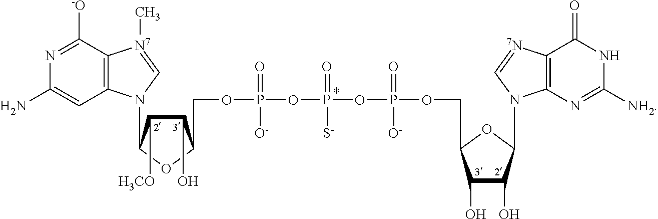

In some embodiments, the 5′ UTR comprises the sequence UUCUUCUGGUCCCCACAGACUCAGAGAGAACCCGCCACC (SEQ ID NO:5). In some embodiments, the 5′ UTR comprises the sequence

GGCGAACUAGUAUUCUUCUGGUCCCCACAGACUCAGAGAG
AACCCGCCACC

In some embodiments, the secretory signal peptide comprises the amino acid sequence MRVMAPRTLILLLSGALALTETWAGS (SEQ ID NO:9). In some embodiments, the polynucleotide sequence encoding the secretory signal peptide comprises the sequence AUGAGAGUGAUGGCCCCCAGAACCCUGAUCCUGCUGCUGUCUGGCGCCCUGGC CCUGACAGAGACAUGGGCCGGAAGC (SEQ ID NO:7). In some embodiments, the at least portion of the transmembrane and cytoplasmic domain of the MHC molecule comprises the amino acid sequence

IVGIVAGLAVLAVVVIGAVVATVMCRRKSSGGKGGSYSQA
ASSDSAQGSDVSLTA

In some embodiments, the polynucleotide sequence encoding the at least portion of the transmembrane and cytoplasmic domain of the MHC molecule comprises the sequence
AUCGUGGGAAUUGUGGCAGGACUGGCAGUGCUGGCCGUGGUGGUGAUCGGAG CCGUGGUGGCUACCGUGAUGUGCAGACGGAAGUCCAGCGGAGGCAAGGGCGGC AGCUACAGCCAGGCCGCCAGCUCUGAUAGCGCCCAGGGCAGCGACGUGUCACU GACAGCC (SEQ ID NO:10). In some embodiments, the 3′ untranslated region of the AES mRNA comprises the sequence CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACU CACCACCUCUGCUAGUUCCAGACACCUCC (SEQ ID NO:15). In some embodiments, the non-coding RNA of the mitochondrially encoded 12S RNA comprises the sequence CAAGCACGCAGCAAUGCAGCUCAAAACGCUUAGCCUAGCCACACCCCCACGGG AAACAGCAGUGAUUAACCUUUAGCAAUAAACGAAAGUUUAACUAAGCUAUAC UAACCCCAGGGUUGGUCAAUUUCGUGCCAGCCACACCG (SEQ ID NO:17). In some embodiments, the 3′ UTR comprises the sequence

CUCGAGCUGGUACUGCAUGCACGCAAUGCUAGCUGCCCCU
UUCCCGUCCUGGGUACCCCGAGUCUCCCCCGACCUCGGGU
CCCAGGUAUGCUCCCACCUCCACCUGCCCCACUCACCACC
UCUGCUAGUUCCAGACACCUCCCAAGCACGCAGCAAUGCA
GCUCAAAACGCUUAGCCUAGCCACACCCCCACGGGAAACA
GCAGUGAUUAACCUUUAGCAAUAAACGAAAGUUUAACUAA
GCUAUACUAACCCCAGGGUUGGUCAAUUUCGUGCCAGCCA
CACCGAGACCUGGUCCAGAGUCGCUAGCCGCGUCGCU

In some embodiments, the poly(A) sequence comprises 120 adenine nucleotides. In some embodiments, the RNA comprises an RNA molecule comprising, in the 5′→3′ direction: the polynucleotide sequence GGCGAACUAGUAUUCUUCUGGUCCCCACAGACUCAGAGAGAACCCGCCACCAU GAGAGUGAUGGCCCCCAGAACCCUGAUCCUGCUGCUGUCUGGCGCCCUGGCCC UGACAGAGACAUGGGCCGGAAGC (SEQ ID NO:1); a polynucleotide sequence encoding the one or more neoepitopes resulting from cancer-specific somatic mutations present in the tumor specimen; and the polynucleotide sequence

(SEQ ID NO: 2)
AUCGUGGGAAUUGUGGCAGGACUGGCAGUGCUGGCCGUGGUGGUGAUC
GGAGCCGUGGUGGCUACCGUGAUGUGCAGACGGAAGUCCAGCGGAGGC
AAGGGCGGCAGCUACAGCCAGGCCGCCAGCUCUGAUAGCGCCCAGGGC
AGCGACGUGUCACUGACAGCCUAGUAACUCGAGCUGGUACUGCAUGCA
CGCAAUGCUAGCUGCCCCUUUCCCGUCCUGGGUACCCCGAGUCUCCCC
CGACCUCGGGUCCCAGGUAUGCUCCCACCUCCACCUGCCCCACUCACC
ACCUCUGCUAGUUCCAGACACCUCCCAAGCACGCAGCAAUGCAGCUCA
AAACGCUUAGCCUAGCCACACCCCCACGGGAAACAGCAGUGAUUAACC
UUUAGCAAUAAACGAAAGUUUAACUAAGCUAUACUAACCCCAGGGUUG
GUCAAUUUCGUGCCAGCCACACCGAGACCUGGUCCAGAGUCGCUAGCC
GCGUCGCU.

In some embodiments, a method for transferring pharmaceutical compositions using peristaltic pumps includes: pumping a first composition from a first container through a first portion of tubing using at least one peristaltic pump head at a first flow rate; pumping a second composition from a second container through a second portion of tubing using at least one peristaltic pump at a second flow rate; and dampening pulses in a fluid flow of the first composition in the first portion of tubing and dampening pulses in a fluid flow of the second composition in the second portion of tubing using a dampener fluidly connected to the first portion of tubing and fluidly connected to the second portion of tubing, wherein the level of pulsation (LoP) of the flow rate of the first flow rate in the first portion of tubing after the dampener is less than 10 and the level of pulsation (LoP) of the flow rate of the second flow rate in the second portion of tubing after the dampener is less than 10.

In some embodiments, a method for manufacturing a pharmaceutical composition including a nucleic acid and one or more lipids includes: pumping a first composition comprising a nucleic acid from a first container through a first portion of tubing using at least one peristaltic pump head at a first flow rate; pumping a second composition comprising one or more lipids from a second container through a second portion of tubing using at least one peristaltic pump head at a second flow rate; dampening pulses in a fluid flow of the first composition in the first portion of tubing and dampening pulses in a fluid flow of the second composition in the second portion of tubing using a dampener fluidly connected to the first portion of tubing and fluidly connected to the second portion of tubing; mixing the first composition comprising the nucleic acid from the first portion of tubing and the second composition comprising the one or more lipids from the second portion of tubing in a mixer fluidly connected to the first portion of tubing and the second portion of tubing; and depositing a composition comprising the nucleic acid and the one or more lipids into a container that is fluidly connected to the mixture.

In some embodiments, the first portion of tubing and the second portion of tubing are configured to be connected to pump heads of the same peristaltic pump. In some embodiments, the first portion of tubing, the second portion of tubing, the dampener, the mixer, and/or the mixture container are made up of single-use materials. the tubing kit or system is an aespetic, closed tubing kit or system; or the methods are performed in an aespetic, closed system.

In some embodiments, a tubing kit for forming a mixture includes: a first portion of tubing configured to be fluidly connected to a first container containing a first composition; a second portion of tubing configured to be fluidly connected to a second container containing a second composition; a tubing dampener comprising an enclosed volume of fluid fluidly connected to the first portion of tubing and the second portion of tubing; a mixer fluidly connected to the first portion of tubing and the second portion of tubing downstream from the fluid dampener and configured to mix the first composition from the first portion of tubing and the second composition from the second portion of tubing; a mixture container fluidly connected to the mixer and configured to collect the mixed first composition and second composition from the mixer, wherein the first portion of tubing is configured to be connected to a first peristaltic pump head upstream the tubing dampener for pumping the first composition from the first container to the mixture container, and the second portion of tubing is configured to be connected to a second peristaltic pump head upstream the tubing dampener for pumping the second composition from the second container to the mixture container.

In some embodiments, a system for forming a pharmaceutical composition or mixture of pharmaceutical compositions includes: a first container containing a first pharmaceutical composition; a second container containing a second pharmaceutical composition; a first portion of tubing fluidly connected to the first container; a second portion of tubing fluidly connected to the second container; a peristaltic pump comprising a first peristaltic pump head connected to the first portion of tubing for pumping the first pharmaceutical composition from the first container and a second peristaltic pump head connected to the second portion of tubing for pumping the second pharmaceutical composition from the second container; a tubing dampener comprising an enclosed volume of fluid fluidly connected to the first portion of tubing and the second portion of tubing downstream from the peristaltic pump; a mixer fluidly connected to the first portion of tubing and the second portion of tubing downstream from the fluid dampener and configured to mix the first pharmaceutical composition from the first portion of tubing and the second pharmaceutical composition from the second portion of tubing; and a mixture container fluidly connected to the mixer and configured to collect the mixed first pharmaceutical composition and second pharmaceutical composition from the mixer.

In some embodiments, a method for transferring pharmaceutical compositions using peristaltic pumps includes: pumping a first composition from a first container through a first portion of tubing using a first peristaltic pump head; pumping a second composition from a second container through a second portion of tubing using a second peristaltic pump head; and dampening pulse in a fluid flow of the first composition in the first portion of tubing downstream the first peristaltic pump head and dampening pulses in a fluid flow of the second composition in the second portion of tubing downstream the second peristaltic pump head using a tubing dampener comprising an enclosed volume of fluid fluidly connected to the first portion of tubing and the second portion of tubing; mixing the first composition from the first portion of tubing and the second composition from the second portion of tubing in a mixer fluidly connected to the first portion of tubing and the second portion of tubing downstream from the fluid dampener; and depositing a composition comprising mixed first and second composition into a container that is fluidly connected to the mixer.

In some embodiments, a tubing kit for forming a mixture includes a first portion of tubing configured to be fluidly connected to a container containing a first composition; a second portion of tubing configured to be fluidly connected to a container containing a second composition; a first dampener fluidly connected to the first portion of tubing; a second dampener fluidly connected to the second portion of tubing; a mixer for mixing the first composition from the first portion of tubing and the second composition from the second portion of tubing; a mixture container for collecting the mixed first composition and second composition from the mixer, wherein the first portion of tubing is configured to be connected to at least one peristaltic pump head for pumping the first composition from the container containing the first composition to the mixture container, and the second portion of tubing is configured to be connected to at least one peristaltic pump head for pumping the second composition from the container containing the second composition to the mixture container. In some embodiments, the first and/or second dampener comprises an enclosed volume of fluid. In some embodiments, the fluid is air. In some embodiments, the first and/or second dampener is a tubing dampener. In some embodiments, the dampener comprises a flexible membrane. In some embodiments, the tubing kit includes a first tee connector that fluidly connects the first dampener, the first portion of tubing, and a first mixer input portion of tubing, wherein the first mixer input portion of tubing fluidly connects to the mixer. In some embodiments, the tubing kit includes a second tee connector that fluidly connects the second dampener, the second portion of tubing, and a second mixer input portion of tubing, wherein the second mixer input portion of tubing fluidly connects to the mixer. In some embodiments, the first portion of tubing comprises a first segment of tubing and a second segment of tubing, wherein the first segment of tubing and the second segment of tubing are fluidly connected in parallel. In some embodiments, the first segment of tubing is configured to be connected to a first peristaltic pump head, and the second segment of tubing is configured to be connected to a second peristaltic pump head. In some embodiments, wherein the second portion of tubing comprises a third segment of tubing and a fourth segment of tubing, wherein the third portion of tubing and the fourth portion of tubing are fluidly connected in parallel. In some embodiments, wherein the third segment of tubing is configured to be connected to a third peristaltic pump head, and the fourth segment of tubing is configured to be connected to a fourth peristaltic pump head. In some embodiments, the mixer comprises an input fluidly connected to the first portion of tubing, an input fluidly connected to the second portion of tubing, and output fluidly connected to the mixture container. In some embodiments, the mixer comprises a Y-connector, a helical mixer, or a static mixer. In some embodiments, the tubing kit includes a first dampener connector that fluidly connects the first portion of tubing to the first dampener and to the mixer and a second dampener connector that fluidly connects the second portion of the tubing to the second dampener and to the mixer. In some embodiments, the mixture container is a bag, vessel, or bottle.

In some embodiments, a pulsation dampener for a fluid pump includes a bioproces sing bag comprising a fluid inlet and a fluid outlet, wherein the fluid inlet is configured to be fluidly connected downstream of a fluid pump; a housing configured to receive the bioproces sing bag, wherein the housing comprises a base and plurality of sidewalls forming a cavity for the bioprocessing bag and at least one sidewall comprises one or more notches configured to provide access to the fluid inlet and fluid outlet of the bioprocessing bag; and a housing lid configured to attach to the plurality of sidewalls and close the housing. In some embodiments, the bioprocessing bag comprises a gas inlet, wherein the gas inlet is configured to be fluidly connected to a gas source. In some embodiments, the one or more notches are configured to provide access to the gas inlet of the bio processing bag. In some embodiments, the base of housing comprises a window. In some embodiments, the window comprises an opening in the base of the housing or a transparent material in the base of the housing. In some embodiments, the housing lid comprises a window. In some embodiments, the window comprises an opening in the housing lid or a transparent material in the housing lid. In some embodiments, the dampener includes a front plate configured to be connected to at least one sidewall of housing and/or the housing lid, wherein the front plate comprises at least one aperture configured to receive the fluid inlet and fluid outlet. In some embodiments, the fluid pump is a cyclic pump. In some embodiments, the cyclic pump is a peristaltic pump. In some embodiments, the fluid outlet is configured to be fluidly connected to a fluid storage container. In some embodiments, the fluid outlet comprises a check valve.

Additional advantages will be readily apparent to those skilled in the art from the following detailed description. The examples and descriptions herein are to be regarded as illustrative in nature and not restrictive.

### BRIEF DESCRIPTION OF THE DRAWINGS

Exemplary embodiments are described with reference to the accompanying figures, in which:

FIG. 1 illustrates an example of an experimental setup to measure flowrates of a peristaltic pump in accordance with some embodiments disclosed herein.

FIG. 2 illustrates the flowrate of water through the peristaltic pump achieved in Experiment 1.

FIG. 3 illustrates an example of an experimental setup to measure flowrates of a peristaltic pump(s) with a dampener in accordance with some embodiments disclosed herein.

FIG. 4 is an image of syringe dampeners with various tee connectors as disclosed herein.

FIG. 5 illustrates the flowrate through the peristaltic pump achieved in Experiment 2.

FIG. 6 illustrates the flowrate through the peristaltic pump achieved in Experiment 4.

FIG. 7 illustrates the flowrate through the peristaltic pump achieved in Experiment 6.

FIG. 8 is an image of a membrane dampener with a tee connector as disclosed herein.

FIG. 9 illustrates an example of a tee connector tubing connector in accordance with some embodiments disclosed herein.

FIG. 10 illustrates an example of a cross tubing connector in accordance with some embodiments disclosed herein.

FIG. 11 illustrates an example of an experimental setup to measure flowrates of a two source peristaltic pump system with dampener(s) in accordance with some embodiments disclosed herein.

FIG. 12 illustrates an example of a dampener loop connecting two separate fluid lines.

FIG. 13 illustrates the flowrate of water through the peristaltic pump system achieved in Experiment 19.

FIG. 14 illustrates the flowrate of water through the peristaltic pump achieved in Experiment 20.

FIG. 15 illustrates an example of a tubing kit in accordance with some embodiments disclosed herein.

FIG. 16 shows the general structure of an exemplary RNA molecule (i.e., a poly-neoepitope RNA). This figure is a schematic illustration of the general structure of the RNA drug substance with constant 5′-cap (beta-S-ARCA (D1)), 5′-and 3′-untranslated regions (hAg-Kozak and FI, respectively), N- and C-terminal fusion tags (sec_{2.0} and MITD, respectively), and poly(A)-tail (A120) as well as tumor-specific sequences encoding the neoepitopes (neol to 10) fused by GS-rich linkers.

FIG. 17 is the ribonucleotide sequence (5′->3′) of the constant region of an exemplary RNA molecule (SEQ ID NO:24). The linkage between the first two G residues is the unusual bond (5′→5′)-pp s p- as shown in FIG. 18 for the 5′ capping structure. The insertion site for patient cancer-specific sequences is between the C131 and A132 residues (marked in bold text). “N” refers to the position of polynucleotide sequence(s) encoding one or more (e.g., 1-20) neoepitopes (separated by optional linkers).

FIG. 18 is the 5′-capping structure beta-S-ARCA(D1) (m₂^{7·2′·O} GppₛpG) used at the 5′ end of the RNA constant regions. The stereogenic P center is Rp-configured in the “D1” isomer. Note: Shown in red are the differences between beta-S-ARCA(D1) and the basic cap structure m⁷GpppG; an —OCH3 group at the C2′ position of the building block m⁷ G and substitution of a non-bridging oxygen at the beta-phosphate by sulphur. Owing to the presence of a stereogenic P center (labelled with *), the phosphorothioate cap analogue beta-S-ARCA exists as two diastereomers. Based on their elution order in reversed-phase high-performance liquid chromatography, these have been designated as 01 and 02.

FIG. 19 is a schematic overview of an HPPD device used in an experiment explained herein.

FIG. 20 illustrates an experimental setup utilizing a glass bottle as an HPPD.

FIG. 21 is a graph depicting the time taken for the glass bottle dampener of FIG. 20 to achieve constant pressure and the time for the same HPPD to dissipate this acquired pressure after the pump has stopped.

FIG. 22 is a graph depicting the displacement of the glass bottle dampener of FIG. 20 at various flow rates over a constant duration.

FIG. 23 is a graph displaying the pressure differential over the glass bottle HPPD device (pressure before versus pressure after the HPPD) as a function of increased air pocket size.

FIG. 24 illustrates the laboratory flask-based HPPD (left) and the same device with a removable liner, permitting the HPPD to be treated theoretically as a single use device (right).

FIG. 25 is a graph depicting the efficiency of the HPPD dampener of FIG. 24 with and without the liner (i.e., bladder dampener).

FIG. 26 illustrates a flexible, single use HPPD design based on a modified bioprocessing bag that includes an elevated inlet tube, to ensure no back washing of the pumped fluid in the event of high backpressure, and an additional third point permitting the insertion of gas to prefill the bag with a gas cushion to improve priming efficiency of the device.

FIG. 27 illustrates the bioprocessing bag dampener with a carton casing.

FIG. 28A is an exploded view of a HPPD in accordance with some embodiments disclosed herein.

FIG. 28B is a housing of a HPPD in accordance with some embodiments disclosed herein.

FIG. 28C is an in-use HPPD in accordance with some embodiments disclosed herein.

FIG. 29A illustrates peristaltic pump experimental setup and flow profile results.

FIG. 29B illustrates a syringe pump experimental setup and flow profile results.

FIG. 29C illustrates a peristaltic pump experimental setup with a HPPD dampener disclosed herein and flow profile results.

FIG. 30 illustrates a commercial Cole-Parmer HPPD.

FIG. 31A illustrates a flow profile of a Cole-Parmer HPPD setup.

FIG. 31B illustrates a flow profile of an HPPD dampener disclosed herein.

FIG. 32 illustrates dead volume of an HPPD dampener disclosed herein and the pressure at the dampener inlet with increasing flow rate.

FIG. 33A illustrates influence of inner diameter of 1.6 mm on pulsation as disclosed herein.

FIG. 33B illustrates influence of inner diameter of 3.2 mm on pulsation as disclosed herein.

FIG. 33C illustrates influence of inner diameter of 6 mm on pulsation as disclosed herein.

FIG. 34A illustrates influence of tubing length of 1 meter on pulsation as disclosed herein.

FIG. 34B illustrates influence of tubing length of 2 meters on pulsation as disclosed herein.

FIG. 34C illustrates influence of tubing length of 20 meters on pulsation as disclosed herein.

### DETAILED DESCRIPTION

Applicants have discovered methods and systems using peristaltic pumps that includes a dampener to drastically reduce the pulsations or oscillations from peristaltic pumps. The methods and systems disclosed herein can minimize the number of parts needed to achieve dampening. In addition, the kits and systems disclosed herein can be a single use, disposable kits and systems, which can provide major benefits to produce, mix, transfer and/or manufacture pharmaceutical compositions and formulations, including, e.g., compositions and formulations comprising RNA and lipids, including lipoplexes or liposomes. In some embodiments disclosed herein, the compositions and formulations comprising RNA and lipids, including lipoplexes or liposomes, are RNA vaccines.

The disclosure also provides a peristaltic pump, dampener and tubing kit system suitable for use with two fluid sources, including, e.g., the pharmaceutical compositions described herein, and in particular, a first pharmaceutical composition comprising RNA, RNA molecules or RNA vaccine, and a second pharmaceutical composition comprising one or more lipids, which can be mixed to create, transfer or manufacture a final pharmaceutical composition comprising RNA-lipoplexes, RNA liposomes or a RNA vaccine. In some embodiments the methods and systems described herein are useful in GMP manufacturing processes which require substantial reduction in the pulsation or oscillations of flow rates normally observed when using peristaltic pumps.

### I. Definitions

Before describing the invention in detail, it is to be understood that the terminology used herein is for the purpose of describing particular embodiments only, and is not intended to be limiting.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a” “an” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes” “including” “comprises” and/or “comprising” when used herein, specify the presence of stated features, integers, steps, operations, elements, components, and/or units but do not preclude the presence or addition of one or more other features, integers, steps, operations, elements, components, units, and/or groups thereof.

Throughout this disclosure, various aspects are presented in a range format. It should be understood that the description in range format is merely for convenience and brevity and should not be construed as an inflexible limitation on the disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed in the disclosure. The upper and lower limits of these smaller ranges may independently be included in the smaller ranges, and are also encompassed in the disclosure, subject to any specifically excluded limit in the stated range. Where the stated range includes one or both of the limits, ranges excluding either or both of those included limits are also included in the disclosure. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1% as understood by a person of skill in the art.

As used herein, a composition refers to any mixture of one or more products, substances, or compounds, including cells. It may be a solution, a suspension, liquid, powder, a paste, aqueous, non-aqueous or any combination thereof.

As used herein, a “peristaltic pump” refers to a type of positive displacement pump that can be used for pumping a variety of fluids. Peristaltic pumps include, but are not limited to, Masterflex Pump (HV-77921-75) and Watson Marlow Flexicon (PD12I). Typically, peristaltic pumps are used in biopharma for two reasons: (1) the system can pump fluid via a closed system (i.e., no exposed pump parts come in contact with the fluid; and (2) the shear stresses are mild.

The term “dampener” refers to any component, device, or mechanism that can reduce pulsations and/or oscillations of the flowrate from a pump, including, e.g., a peristaltic pump.

The term “tubing kit” refers to an assembly of tubes/tubing and other components that can interact with the tubes/tubing.

The term “pharmaceutical composition” or “pharmaceutical formulation” or “pharmaceutical compositions and formulations” refers to a preparation which is in such form as to permit the biological activity of an active ingredient contained therein to be effective, and which contains no additional components which are unacceptably toxic to a subject to which the pharmaceutical composition would be administered. Such formulations are sterile. “Pharmaceutically acceptable” excipients (vehicles, additives) are those which can reasonably be administered to a subject mammal to provide an effective dose of the active ingredient employed. Pharmaceutically acceptable carrier includes, but is not limited to, a buffer, excipient, stabilizer, or preservative. In some embodiments described herein, the pharmaceutical compositions comprise a nucleic acid (including, e.g., RNA, mRNA or RNA vaccine) and/or one or more lipids (including, e.g., a cationic lipid and/or a neutral “helper” lipid).

An “individual”, “subject” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

The term “nucleic acids” or “nucleic acid molecule” or “polynucleotide” includes any compound and/or substance that comprises a polymer of nucleotides. Each nucleotide is composed of a base, specifically a purine or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA), genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “RNA” or “RNA molecule” relates to a molecule comprising ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

The term “RNA” also includes and preferably relates to “mRNA” which means “messenger RNA” and relates to a “transcript” which may be produced using DNA as template and encodes a peptide or protein. mRNA typically comprises a 5′ non translated region (5′-UTR), a protein or peptide coding region and a 3′ non translated region (3′-UTR). mRNA has a limited halftime in cells and in vitro. mRNA can be produced by in vitro transcription using a DNA template or chemical synthesis. The in vitro transcription methodology is known to the skilled person. For example, there is a variety of in vitro transcription kits commercially available.

An “RNA vaccine” as used herein refers to an RNA, RNA polynucleotide or RNA molecule which encodes one or more antigens and induces an immune response (e.g., protective immunity against the antigen) when administered to a subject or individual. Several RNA vaccines have been described. See, e.g., Pardi et al. “mRNA vaccines—a new era in vaccinology”. Nat Rev Drug Discov 17, 261-279 (2018). https://doi.org/10.1038/nrd.2017.243.

The term, “lipoplex” or “RNA lipoplex” refers to a complex of lipids and nucleic acids such as RNA. Lipoplexes are formed spontaneously when cationic lipids and/or liposomes, which often also include a neutral “helper” lipid, are mixed with nucleic acids.

Where the disclosure refers to a charge such as a positive charge, negative charge or neutral charge or a cationic compound, negative compound or neutral compound this generally refers to the charge mentioned is present at a selected pH, such as a physiological pH. For example, the term “cationic lipid” means a lipid having a net positive charge at a selected pH, such as a physiological pH. The term “neutral lipid” means a lipid having no net positive or negative charge and can be present in the form of a non-charge or a neutral amphoteric ion at a selected pH, such as a physiological pH. By “physiological pH” herein is meant a pH of about 7.5.

The lipid carriers described herein for use in the present invention include any substances or vehicles with which RNA can be associated, e.g. by forming complexes with the RNA or forming vesicles in which the RNA is enclosed or encapsulated. This may result in increased stability of the RNA compared to naked RNA. In particular, stability of the RNA in blood may be increased.

Cationic lipids, cationic polymers and other substances with positive charges may form complexes with negatively charged nucleic acids. These cationic molecules can be used to complex nucleic acids, thereby forming e.g. so-called lipoplexes or polyplexes, respectively.

Liposomes are microscopic lipidic vesicles often having one or more bilayers of a vesicle-forming lipid, such as a phospholipid, and are capable of encapsulating a drug or nucleic acid molecule, such as RNA. Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation and the selection of the type of vesicles to be used will depend on the preferred mode of administration. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state.

For formation of liposomes, any suitable method of forming liposomes can be used so long as it provides liposomes suitable for manufacturing the envisaged RNA lipoplexes. Liposomes may be formed using standard methods such as the reverse evaporation method (REV), the ethanol injection method, the dehydration-rehydration method (DRV), sonication or other suitable methods. After liposome formation, the liposomes can be sized to obtain a population of liposomes having a substantially homogeneous size range.

Bilayer-forming lipids have typically two hydrocarbon chains, particularly acyl chains, and a head group, either polar or nonpolar. Bilayer-forming lipids are either composed of naturally-occurring lipids or of synthetic origin, including the phospholipids, such as phosphatidylcholine, phosphatidylethanolamine, phosphatide acid, phosphatidylinositol, and sphingomyelin, where the two hydrocarbon chains are typically between about 14-22 carbon atoms in length, and have varying degrees of unsaturation. Other suitable lipids for use in the composition of the present invention include glycolipids and sterols such as cholesterol and its various analogs which can also be used in the liposomes.

Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. Most preferred is DOTMA.

### II. Overview

Provided herein are peristaltic pump systems that include a dampener for reducing the pulsations or oscillations of the flowrate from the peristaltic pumps in the system, and methods of use of these systems to produce, mix, transfer and/or manufacture pharmaceutical compositions and formulations, including, e.g., compositions and formulations comprising RNA and lipids, including lipoplexes or liposomes. In some embodiments, the pharmaceutical compositions and formulations comprise an RNA, RNA molecule or RNA vaccine.

The disclosure also provides a peristaltic pump, dampener and tubing kit system suitable for use with two fluid sources, including, e.g., the pharmaceutical compositions described herein, and in particular, a first pharmaceutical composition comprising RNA, RNA molecules or RNA vaccine, and a second pharmaceutical composition comprising one or more lipids described herein, which can be mixed or transferred to create or manufacture a final pharmaceutical composition comprising RNA-lipoplexes, RNA liposomes or a RNA vaccine.

### III. Peristaltic Pumps, Dampeners & Tubing Kit Systems

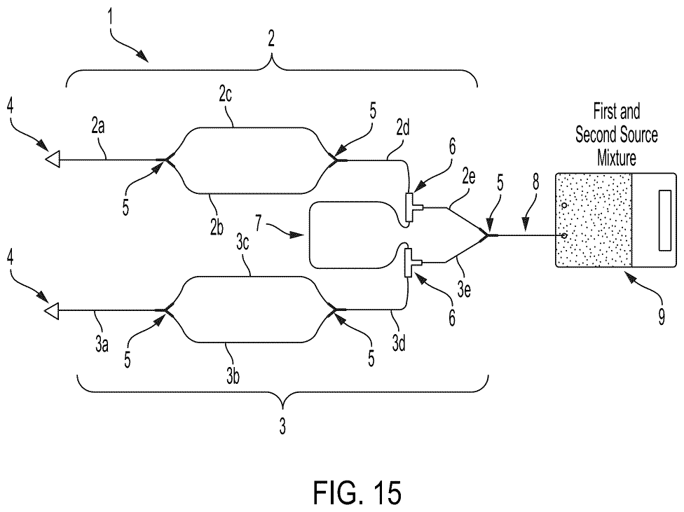

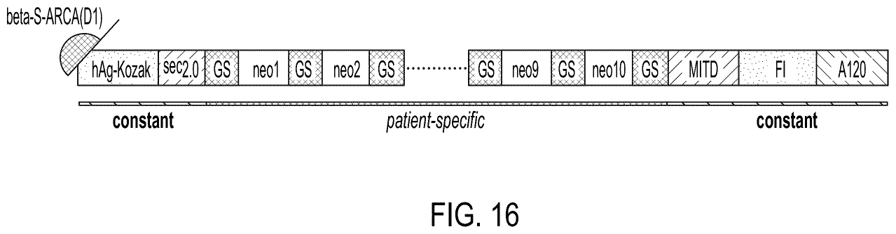

Provided herein are tubing kits for use with a peristaltic pump system. In some embodiments, the peristaltic pump can be a Masterflex pump (HV-77921-75) which has four rollers and can be adapted to have multiple number of pump heads. In some embodiments, the peristaltic pump can be a Watson Marlow. In some embodiments, the tubing kit can be used for forming a pharmaceutical composition, formulation and/or mixture. In some embodiments, the tubing kit can be used for mixing in-line to form a pharmaceutical composition, formulation and/or mixture. In some embodiments, a pharmaceutical composition, formulation and/or mixture can be formed by mixing a first pharmaceutical composition with a second pharmaceutical composition. FIG. 15 illustrates an example of tubing kit 1. The tubing kit can include a first portion of tubing configured to be fluidly connected to a container. An example of a first portion of tubing is shown in FIG. 15 as portion of tubing 2.

Portion of tubing 2 can include inlet tubing 2a, pump tubing 2b and 2c, pump outlet tubing 2d, and post-dampener tubing 2e. Inlet tubing 2a can be fluidly connected to a fluid source or container. In some embodiments, the first portion of tubing can include a connector 4 for fluidly connecting the first portion of tubing to a fluid source or container. In some embodiments, the connector fluidly connecting the first portion of tubing to a fluid source or container can be an aseptic connector. In some embodiments, any connector from container to tubing can work. In some embodiments, the container can include a first fluid or composition. In some embodiments, the first composition can be a first pharmaceutical composition, including, e.g., a lipid or RNA containing composition. In some embodiments, the container can be a fluid container such as a bag, bottle, and/or vessel.

The first portion of tubing can include a first segment of tubing and a second segment of tubing. In some embodiments, the first segment of tubing and the second segment of tubing are fluidly connected in parallel. For example, first portion of tubing includes pump tubing 2b and pump tubing 2c as shown in FIG. 15. In some embodiments, the first segment of tubing is configured to be connected or fitted to a first head of a peristaltic pump and the second segment of tubing is configured to be connected or fitted to a second head of the peristaltic pump.

In some embodiments, inlet tubing 2a can be fluidly connected to pump tubing 2b and 2c through a connector 5. In some embodiments, the connector fluidly connecting the inlet tubing to the pump tubing is a Y-connector or a mixer (e.g., static, helical, etc.). In some embodiments, the pump tubing does not need to be split into parallel pump tubes as shown in FIG. 15. As such, a connector may not be necessary and the inlet tubing and pump tubing can be one in the same. In some embodiments, the first portion of tubing can be configured to be connected or fitted to a peristaltic pump. In some embodiments, the peristaltic pump can be a multi-head peristaltic pump such as a dual head peristaltic pump. In some embodiments, the pump tubing 2b and 2c are connected or fitted to the first peristaltic pump. For example, if the peristaltic pump is a dual head peristaltic pump, pump tubing 2b can be connected or fitted to one pump head and pump tubing 2ccan be connected or fitted to another pump head. The same logic can be employed to pumps with more than two heads. In embodiments, the first peristaltic pump is a single head peristaltic pump. As such, only one of pump tubing 2b or 2c are connected or fitted to the first peristaltic pump or the single inlet tubing/pump tubing can be connected or fitted to the first peristaltic pump.

In some embodiments, pump tubing or inlet/pump tubing can be fluidly connected to pump outlet tubing. In some embodiments, pump tubing 2b and 2c are fluidly connected to pump outlet tubing 2d through connector 5 (e.g., a Y-connector). In some embodiment, the pump tubing does not need to be split into parallel pump tubes. Thus, a connector after the pump may not be necessary and the pump tubing and the pump outlet tubing can be one in the same. For example, tubing can be the inlet, pump, and pump outlet tubing.

In some embodiments, the tubing kit can include a dampener fluidly connected to the first portion of tubing. The first portion of the tubing can be fluidly connected to the dampener through a connector. In some embodiments, such a connector can be a tee connector, a 4-way connector, or various other types of connectors. FIG. 15 illustrates first portion of tubing 2 fluidly connected to dampener 7 through connector 6. Specifically, pump outlet tubing 2d is fluidly connected to dampener 7 through connector 6. In some embodiments, first portion of tubing 2 can be fluidly connected to its own first dampener. In some embodiments, the dampener can also be fluidly connected to post-dampener tubing. In some embodiments, the pump outlet tubing is fluidly connected to post-dampener tubing through a connector (e.g., connector 5 as shown in FIG. 15). In some embodiments, the dampener, the pump outlet tubing, the connector, and the post-dampener tubing are fluidly connected.

The tubing kit can also include a second portion of tubing configured to be fluidly connected to a container. An example of a second portion of tubing is shown in FIG. 15 as portion of tubing 3. In some embodiments, the first portion of tubing and the second portion of tubing are fluidly connected in parallel.

Portion of tubing 3 can include inlet tubing 3a, pump tubing 3b and 3c, pump outlet tubing 3d, and post-dampener tubing 3e. Inlet tubing 3a can be fluidly connected to a fluid source or container. In some embodiments, the second portion of tubing can include a connector 4 for fluidly connecting the second portion of tubing to a fluid source or container. In some embodiments, the connector fluidly connecting the second portion of tubing to a fluid source or container can be an aseptic connector. In some embodiments, any connector from container to tubing can work. In some embodiments, the container can include a second fluid or composition different from the first fluid or composition. In some embodiments, the container can include a fluid or composition that is the same as the first fluid or composition. In some embodiments, the second composition can be a second pharmaceutical composition including, e.g., a lipid or RNA containing composition. In some embodiments, the container can be a fluid container such as a bag, bottle, and/or vessel.

The second portion of tubing can include a third segment of tubing and a fourth segment of tubing. In some embodiments, the third segment of tubing and the fourth segment of tubing are fluidly connected in parallel. For example, second portion of tubing includes pump tubing 3b and pump tubing 3c as shown in FIG. 15. In some embodiments, the third segment of tubing is configured to be connected or fitted to a first head of a peristaltic pump and the fourth segment of tubing is configured to be connected or fitted to a second head of the peristaltic pump. In some embodiments, the peristaltic pump connected or fitted to the second portion of tubing is different than the peristaltic pump connected or fitted to the first portion of tubing. In some embodiments, the same peristaltic pump is connected or fitted to the first portion of tubing and the second portion of tubing. As such, the first portion of tubing can be connected or fitted to a first head or heads of the peristaltic pump and the second portion of tubing can be connected or fitted to a second head or heads of the peristaltic pump.

In some embodiments, inlet tubing 3a can be fluidly connected to pump tubing 3b and 3c through a connector 5. In some embodiments, the connector fluidly connecting the inlet tubing to the pump tubing is a Y-connector or a mixer (e.g., static, helical, etc.). In some embodiments, the pump tubing does not need to be split into parallel pump tubes as shown in FIG. 15. As such, a connector may not be necessary and the inlet tubing and pump tubing can be one in the same. In some embodiments, the second portion of tubing can be configured to be connected or fitted to a second peristaltic pump. In some embodiments, the peristaltic pump can be a multi-head peristaltic pump such as a dual head peristaltic pump. In some embodiments, the pump tubing 3b and 3c are connected or fitted to the second peristaltic pump. For example, if the peristaltic pump is a dual head peristaltic pump, pump tubing 3b can be connected or fitted to one pump head and pump tubing 3c can be connected or fitted to another pump head. The same logic can be employed to pumps with more than two heads. In some embodiments, the second peristaltic pump is a single head peristaltic pump. As such, only one of pump tubing 3b or 3c are connected or fitted to the second peristaltic pump or the single inlet tubing/pump tubing can be connected or fitted to the second peristaltic pump.

In some embodiments, the first peristaltic pump is the same pump as the second peristaltic pump and the various portions of tubing are configured to be connected or fitted to separate heads of the pump. As such, pump tubing 2b can be connected or fitted to a first pump head, pump tubing 2c can be connected or fitted to a second pump head, pump tubing 3c can be connected or fitted to a third pump head, and pump tubing 3b can be connected or fitted to a fourth pump head. Or, only one of pump tubing 2b or 2c can be connected or fitted to a first pump head or the first single inlet tubing/pump tubing can be connected or fitted to a first pump head and only one of pump tubing 3b or 3c can be connected or fitted to a second pump head or the second single inlet tubing/pump tubing can be connected or fitted to a second pump head.

In some embodiments, pump tubing or inlet/pump tubing can be fluidly connected to pump outlet tubing. In some embodiments, pump tubing 3b and 3c are fluidly connected to pump outlet tubing 3d through connector 5 (e.g., a Y-connector). In some embodiments, the pump tubing does not need to be split into parallel pump tubes. Thus, a connector after the pump may not be necessary and the pump tubing and the pump outlet tubing can be one in the same. For example, tubing can be the inlet, pump, and pump outlet tubing.

In some embodiments, the tubing kit can include a dampener fluidly connected to the second portion of tubing. In some embodiments, the dampener is fluidly connected to the first portion of tubing and fluidly connected to the second portion of tubing. In some embodiments, the dampener is fluidly connected to the first portion of tubing and/or the second portion of tubing through a connector such as a tee connector, 4-way connector, etc.

The dampener can be any device in which the dampening is performed by an enclosed volume of fluid. In some embodiments, the volume of fluid in the dampener can depend on the flowrate and/or the exposed surface area. In some embodiments, the dampener dampens pulsations by an enclosed volume of air. In some embodiments, the dampener can be a syringe dampener, a membrane dampener (e.g., flexible membrane dampener), or a tubing dampener. In some embodiments, the tubing dampener can be a dead ended tubing dampener such that one end of the dampener is fluidly connected to the first portion of tubing or the second portion of tubing and the other end of the dampener is closed. In some embodiments, the other end of the dampener can be closed by a clamp, end cap, connected to another dampening line, or another part capable of enclosing gas from the environment. In some embodiments, the tubing dampener is made out of silicone tubing.

In some embodiments, one end of the tubing dampener is fluidly connected to the first portion of tubing and the other end of the tubing dampener is fluidly connected to the second portion of tubing, thereby forming a loop tubing dampener. In some embodiments, the loop tubing dampener is above the first portion of tubing and the second portion of tubing such that minimal fluid from the first and/or second portion of tubing does not enter the dampener and air remains in the dampener. For example, if the solution is flowing horizontally on a surface, the dampener can be place vertically at a height greater than the intended fluid path, such that an air pocket stays above the liquid level during pumping. In some embodiments, the loop tubing dampener can be placed or mounted above the first portion of tubing and the second portion of tubing. In some embodiments, the loop tubing dampener can be mounted on a horizontal bar or attached (i.e., taped) above the first portion of tubing and the second portion of tubing.

The second portion of the tubing can be fluidly connected to the dampener through a connector. In some embodiments, such a connector can be a tee connector, a 4-way connector, or various other types of connectors. FIG. 15 illustrates second portion of tubing 3 fluidly connected to dampener 7 through connector 6. Specifically, pump outlet tubing 3d is fluidly connected to dampener 7 through connector 6. In some embodiments, second portion of tubing 3 can be fluidly connected to its own second dampener. In some embodiments, pump outlet tubing 2d is fluidly connected to its own dampener and pump outlet tubing 3d is connected to its own different dampener. In some embodiments, the dampener can also be fluidly connected to post-dampener tubing. In some embodiments, the pump outlet tubing is fluidly connected to post-dampener tubing through a connector (e.g., connector 5 as shown in FIG. 15). In some embodiments, the dampener, the pump outlet tubing, the connector, and the post-dampener tubing are fluidly connected.

In some embodiments, the tubing kits disclosed herein can include a mixer for mixing the first fluid or composition from the first portion of tubing and a second fluid or composition from the second portion of tubing. As such, the first portion of tubing and the second portion of tubing can be fluidly connected to the mixer. In some embodiments, the first portion of tubing and the second portion of tubing are fluidly connected to the mixer downstream from the dampener. In some embodiments, the post-dampener tubing (i.e., a first mixer input portion of tubing) of the first portion of tubing and the post-dampener tubing (i.e., a second mixer input portion of tubing) of the second portion of tubing are fluidly connected to the mixer. In some embodiments, a connector (e.g., connector 6) fluidly connects the dampener, the first portion of tubing, and a first mixer input portion of tubing. In some embodiments, a connector (e.g., connector 6) fluidly connects the dampener, the second portion of tubing, and a second mixer input portion of tubing. In some embodiments, a first dampener connector fluidly connects the first portion of tubing to the dampener and to the mixer and a second dampener connector fluidly connects the second portion of tubing to the dampener and to the mixer.

In some embodiments, the mixer includes an input fluidly connected to the first portion of tubing, an input fluidly connected to the second portion of tubing, and an output. In some embodiments, the mixer can be a Y connector, a helical mixer, or a static mixer. In some embodiments, the output can be fluidly connected to tubing (e.g., output tubing 8). In some embodiments, the mixer includes an output fluidly connected to a mixture container (e.g., first and second source mixture container 9). The mixture container can collect the mixed first fluid or composition and the second fluid or composition from the mixer. In some embodiments, the mixture container can be a fluid container such as a bag, bottle, and/or vessel.

In some embodiments, the first portion of tubing is configured to be connected or fitted to a first peristaltic pump or pump head for pumping a first fluid or composition from a container to the mixture container. In some embodiments, the second portion of tubing is configured to be connected or fitted to a second peristaltic pump or pump head for pumping a second fluid or composition from a container to the mixture container. In some embodiments, the fluid or compositions pumped through the tubing kits are pharmaceutical compositions including, e.g., a lipid or RNA containing composition. The pharmaceutical compositions disclosed herein can include nucleic acids (including, e.g, RNA or mRNA), one or more lipids, proteins, buffers, small molecules, amino acids, and/or polypeptides. In some embodiments, the nucleic acids can be RNA (including, e.g., mRNA) and/or DNA. In some embodiments, the one or more lipids can be in the form of liposomes or lipoplexes. In some embodiments, the pharmaceutical compositions can be components of a personalized cancer vaccine or RNA vaccine, including nucleic acids and lipids together forming lipoplexes.

The tubing kits, methods and/or systems disclosed herein include a dampener for reducing the pulsations or oscillations of the flowrate from the peristaltic pumps with fluids coming from one or two sources. In some embodiments, the level of pulsation (“LoP”) of the tubing kits, methods and/or systems disclosed herein used with peristaltic pumps with fluids coming from one or two sources is less than about 40, less than about 35, less than about 30, less than about 25, less than about 20, less than about 15, less than about 12, less than about less than about 9, less than about 8, less than about 7, less than about 6, less than about 5, less than about 4, less than about 3, less than about 2, or less than about 1. In some embodiments, the level of pulsation (“LoP”) of the tubing kits methods and/or systems disclosed herein used with peristaltic pumps with fluids coming from one or two sources is between about 7 and about 40 or between about 10 and about 20. In some embodiments, the level of pulsation (“LoP”) of the tubing kits methods and/or systems disclosed herein used with peristaltic pumps with fluids coming from one or two sources can be about 7, about 8, about about 15, about 20 or about 25.

In some embodiments, the level of reduction in the pulsation (“LoP”) of peristaltic pumps with fluids coming from one or two sources using the tubing kits, methods and/or systems disclosed herein is about 98%, about 95%, about 90%, about 85%, about 80%, about 75%, about 70%, about 65%, about 60%, about 55%, about 50%, about 45%, about 40%, about 35%, about 30%, about 25%, or about 20% as compared to the LoP of peristaltic pumps with fluids coming from one or two sources lacking a dampener as described herein.

Although most of the examples and description discuss using the dampener for pharmaceutical compositions and formulations, the dampeners and pump systems disclosed herein are not limited to use in pharmaceutical compositions and formulations. For example, the pump systems disclosed herein can be used for filling operations or simply moving solutions between vessels using peristaltic pumps where it may be advantageous to have a consistent flow rate. The systems disclosed herein can be used with any pump besides a peristaltic pump including, but not limited to, piston, diaphragm, screw, etc; with any flow rate; with any sort of dampener; and any tubing dimensions and tubing materials (e.g., pipes, plastic, stainless). In some embodiments, because the tubing system is product contacting, the tubing system can be sterilized prior to sue. Such sterilization can be done with autoclave, gamma ray, etc.

### IV. Hydropneumatic Pulsation Dampener

As explained above, when using cyclic pumping systems such as peristaltic pumps, fluctuations in pressure and in flow rate may occur in the fluidic path downstream the pump. This so-called pulsation can be reduced by using different technical solutions including hydropneumatic pulsation dampeners (HPPD), which can absorb the pulsations by the compression of a trapped fluid (e.g., gaseous) cushion resulting in a reduced pulsation and a steady, smooth fluid flow.

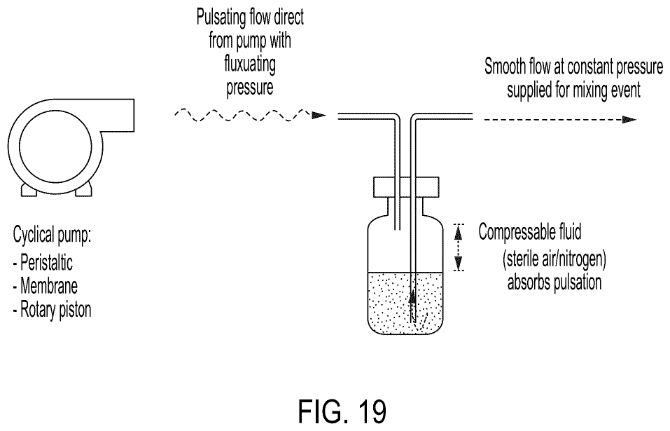

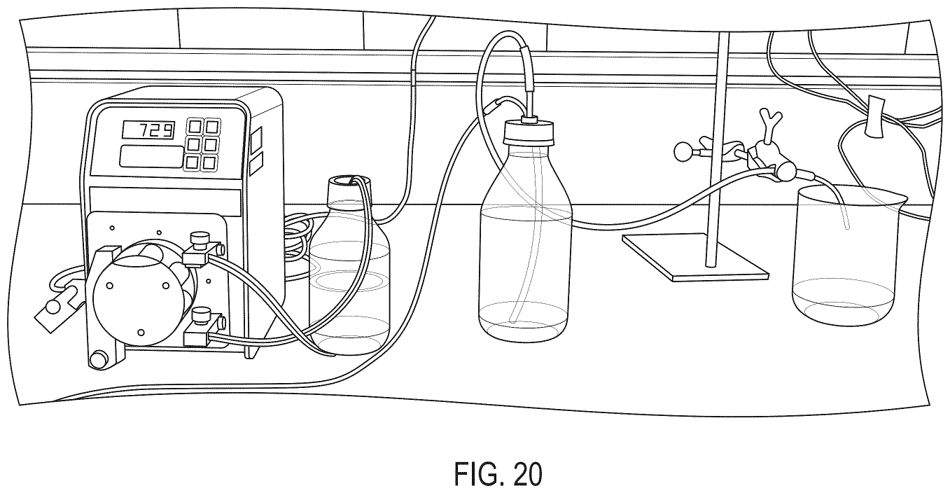

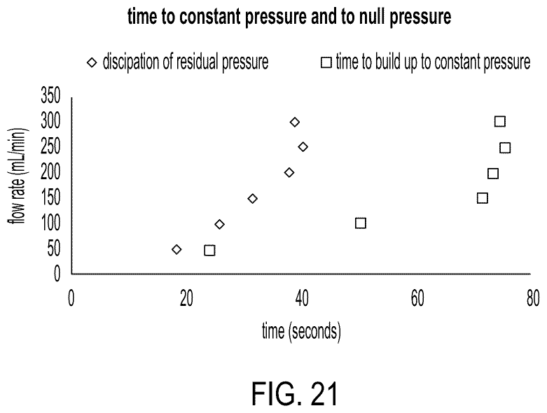

In an initial experiment, this concept was shown to be effective using a glass bottle with an inlet and outlet submerged in the pumped fluid (e.g., water) to entrap a compressible gas (e.g., air) to absorb the pulsation. This concept is shown in FIG. 19. To simulate a sterile manufacturing scenario, the HPPD made from a 500 mL Laboratory bottle was tested at various flow rates and evaluated for the amount of time required to run up to constant pressure and run down from the pressure following stoppage of the pump (i.e., time required to normalize fluid path pressure). FIG. 20 illustrates the initial HDDP setup consisting of a fluid (water) reservoir, peristaltic pump, 500 mL laboratory flask closed with a cap consisting of two ports (one of which is submerged in the pumpable fluid), and a collection vessel at the outlet of the fluid path. As shown in FIG. 21, at lower flow rates, the time build up pressure increased rapidly until a flow rate of approximately 150 mL/min, at which the run up and run down time remained comparable at higher flow rates. This was about 75 and 40 seconds to build up to constant pressure and to dissipate back to zero, respectively.

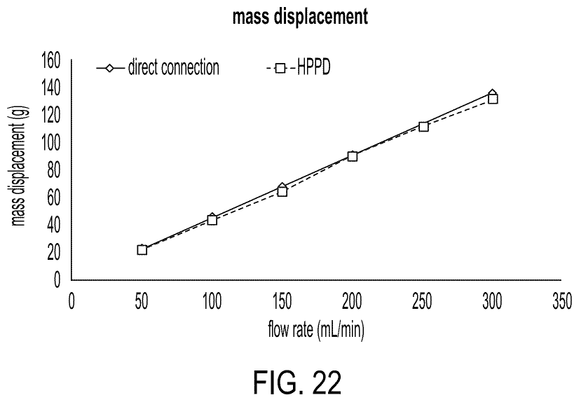

The effect of residual backpressure on the displacement rate was also measured for this glass bottle dampener. FIG. 22 confirms that, though some significant time is required to pressurize and depressurize the HPPD system, the actual displacement of the HPPD-integrated system is constant and equivalent to a simple pump-to-outlet setup.

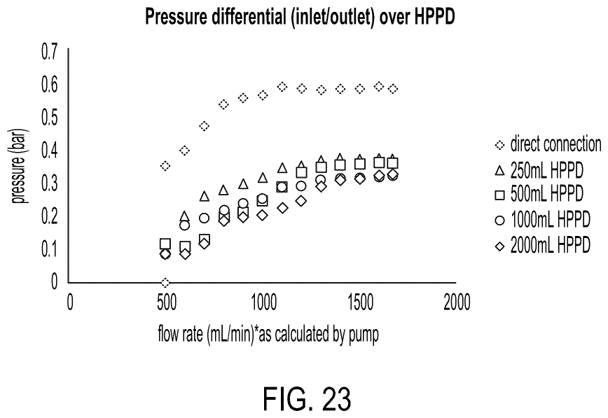

The effect of the bottle size on pressure differential was also investigated. Specifically, the effect of air pocket size was investigated by pumping at a constant rate through HPPDs created using laboratory bottles from 250 to 2000 mL in size. FIG. 23 demonstrates that smaller air volumes result in higher back pressure at inlet but lower pressure losses over the HPPD device overall relative to the direct connection of the pump to the outlet. It was observed that this pressure loss did not affect the smoothness of the flow at the outlet.

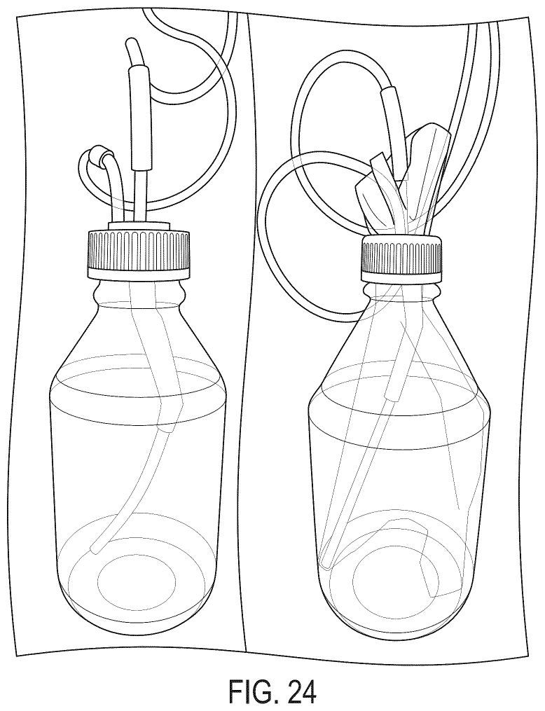

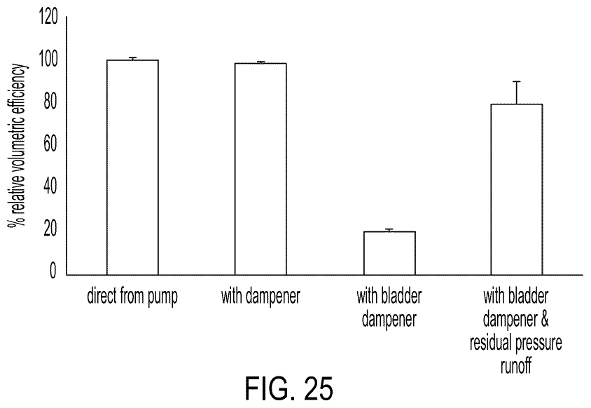

To test the concept for a single use design of the HPPD, a liner was inserted into the laboratory flask, thereby permitting the contact surface and fluid paths to be exchanged after each use. This is shown in FIG. 24. It was seen that the addition of this liner significantly reduced the throughput of the HPPD due to the increased malleability of the flexible liner, as shown in FIG. 25. The liner dampener (i.e., bladder dampener) also worked well with respect to the normalization of the fluid flow and was advantageous since it ensured that the fluid flow would not come into contact with the air during the pumping process. However, the significant reduction in pumping efficiency in the form of residual pressure and fluid runoff after the pump was stopped caused a significant reduction in efficiency to the system.

Accordingly, the glass bottle dampener demonstrated a successful reduction of pulsation. In addition, it had the advantage that relatively large commonly available laboratory bottles can result in a large dead volume and large air pocket which can increase compressibility of the system and thus reduce the outlet pressure through energy losses. The disadvantages were that, without a liner, the glass bottle is not single-use compatible and requires prior assembly. In addition, long priming and run-off times were needed to equilibrate and stop the system, resulting in a high dead volume.

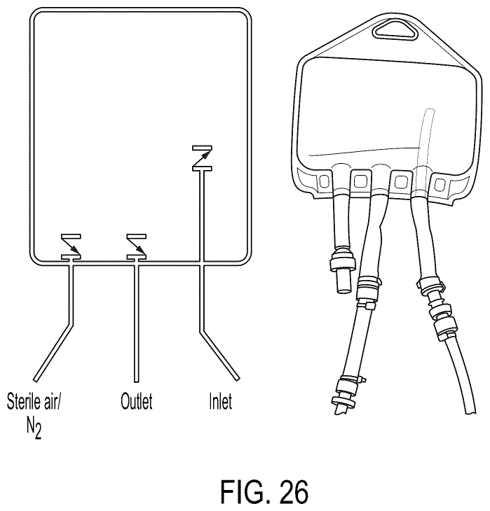

To further reduce the dead volume and to achieve a robust single-use Good Manufacturing Process (GMP) design, the glass bottle was exchanged for a bioprocessing bag, such as a 50 mL FLEXBOY® bioprocessing bag shown in FIG. 26. The FLEXBOY® bag can be modified to include an elevated inlet tube. The inlet of this inlet tube can be located towards the middle of the bag away from the perimeter of the bag as shown in FIG. 26. In contrast, the gas inlet (e.g., sterile air, nitrogen gas) and outlet can be located at the perimeter of the bag. Having the inlet tube to the bioprocessing bag be located towards the middle of the bag can help ensure no back washing of the pumped fluid in the event of high backpressure. In addition, the gas inlet can permit the insertion of gas to prefill the bag with a gas cushion to improve priming efficiency of the HPPD.

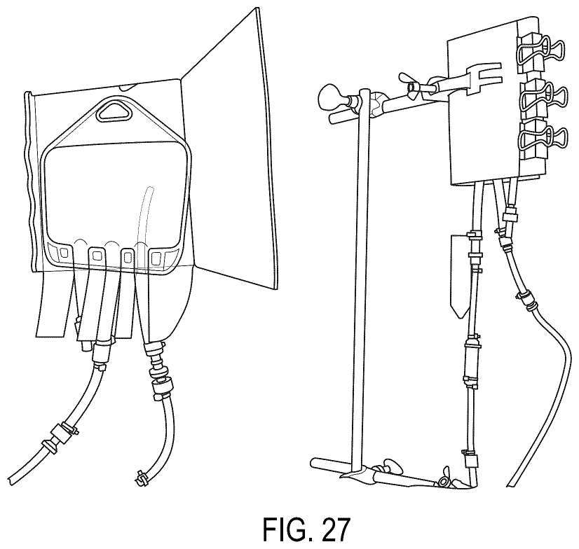

A prototype casing was also made out of carton as shown in FIG. 27 to add rigidity to the bag and minimize its expansion with increasing pump flow rate and pressure, thereby improving the through-flow of the pumped fluid. Specifically, a FLEXBOY® bag was fixed in a carton casing as shown in FIG. 27. This helped ensure that the bag could not expand any further and a gas cushion could be created. It was found that this can help lead to a reduction in dead volume and a shorter primer time. However, due to the weak material of the carton, the HPPD was only stable up to a certain pressure. As such, Applicant discovered a replacement for the carton with a more rigid housing.

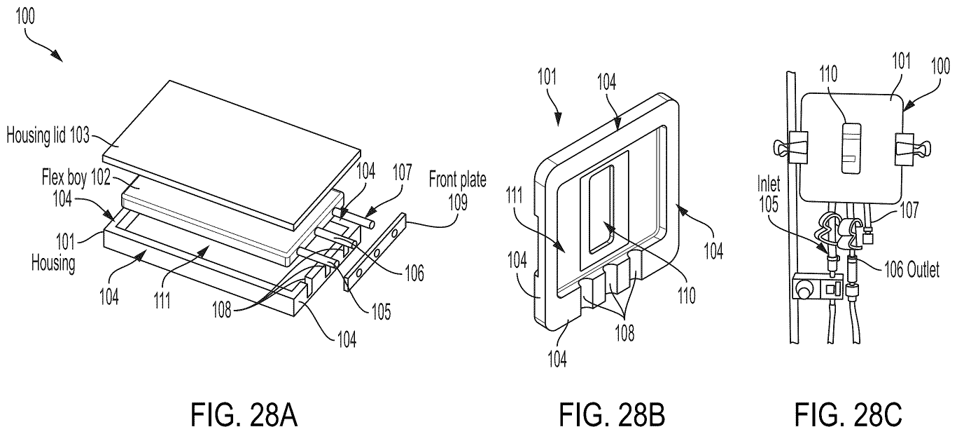

Specifically, FIG. 28A illustrates an exploded view of a pulsation dampener 100 for a fluid pump. In some embodiments, the pulsation dampener can include a bioprocessing bag 102. The bioprocessing bag can be a FLEXBOY® bag. For example, the bioprocessing bag can be a 50 mL FLEXBOY® bag, but other sized bioprocessing bags (e.g., anywhere between 5 mL and 50 L) can also be used. The bioprocessing bag can include a fluid inlet 105 and a fluid outlet 106. In some embodiments, the fluid inlet and/or fluid outlet can be fluidly connected to tubing. In some embodiments, the fluid inlet and/or fluid outlet can include the tubing itself. In some embodiments, the fluid inlet and/or fluid outlet or fluid tubing connected to the fluid inlet/outlet can be located towards the middle of the bag away from the perimeter of the bag as shown in FIG. 26. In some embodiments, the fluid inlet can be fluidly connected downstream of a fluid pump such as a cyclic pump (e.g., peristaltic pump). In some embodiments, the fluid outlet can be fluidly connected to a fluid storage container. In some embodiments, the fluid outlet can include a check valve. In some embodiments, the check valve can have a breakthrough resistance of about 0.05-0.5 bar, about 0.05-0.4 bar, about 0.05-0.3 bar, about 0.1-0.2 bar, or about 0.14 bar.

In some embodiments, the bioprocessing bag can include a gas inlet 107. The gas inlet can be configured to be fluidly connected to a gas source. In some embodiments, the gas source is air (e.g., sterile air) and/or nitrogen. The gas inlet can provide gas to the bioprocessing bag such that the bag includes a gaseous cushion for pulsation dampening.

In some embodiments, the pulsation dampener 100 can include a housing 101. FIG. 28B illustrates housing 101 without the other components of the pulsation dampener. The housing can be configured to receive the bioprocessing bag. In some embodiments, the housing can include a base 111. The base can be a base plate. In some embodiments, the housing can include a plurality of sidewalls 104. In some embodiments, the plurality of sidewalls can extend away from the base along the base's perimeter. In some embodiments, the plurality of sidewalls can be connected to the perimeter of the base. In some embodiments, the plurality of sidewalls and the base can be a single integral component. In some embodiments, the base and plurality of sidewalls can form a cavity which is configured to receive/hold the bioprocessing bag.

In some embodiments, at least one sidewall of the housing can have one or more notches 108. As shown in FIG. 28B, housing 101 includes three notches 108 in sidewall 104. The notch or notches can be an indent or recess into the at least one sidewall of the housing. In some embodiments, the one or more notches can be configured to provide access to the fluid inlet, the fluid outlet, and/or gas inlet of the bioprocessing bag. In some embodiments, the one or more notches can be configured to receive the fluid inlet, the fluid outlet, and/or gas inlet of the bioprocessing bag. As described in more detail below, the housing can be closed via a housing lid. Thus, any fluid or fluid tubing that accesses the bioprocessing bag can enter/exit through the one or more notches in the at least one sidewall of the housing.

In some embodiments, the housing can include a window 110. In some embodiments, the base of the housing can include a window. In some embodiments, the window can be an opening in the base of the housing. In some embodiments, the window can be a transparent material (e.g., glass or clear plastic) in the base of the housing. The window can permit visual inspection of the bioproces sing bag during use.

In some embodiments, dampener 100 can include a housing lid 103. The housing can be configured to close the housing such that the bioprocessing bag is encased by the housing and housing lid. In some embodiments, the housing lid is configured to be attached to the plurality of sidewalls of the housing. In some embodiments, the housing lid can be attached to the housing via any attachment mechanism (e.g., adhesives, screws, nails, bolts, Velcro, clips (as shown in FIG. 28C), locking mechanisms among others). In some embodiments, the housing lid can be attached to at least one sidewall such that the housing lid acts as a door (i.e., hinge mechanism) for the housing. In some embodiments, the housing lid can include a window. In some embodiments, the window can be an opening in the housing lid. Similar to any window in the base of the housing, the window can be a transparent material (e.g., glass or clear plastic) in the base of the housing.

In some embodiments, dampener 100 can include front plate 109. In some embodiments, the front plate can be connected to at least one sidewall 104 of housing 101 and/or housing lid 103. The front plate can include at least one aperture configured to receive the fluid inlet, the fluid outlet, and/or gas inlet of the bioprocessing bag. The front plate can be included to ensure that any inlet/outlet of the bioprocessing bag or corresponding tubing is kept in position within the housing of the dampener 100. In some embodiments, the front plate can be included to ensure that the inlet/outlet of the bioprocessing bag or corresponding tubing is under constant homogeneous pressure throughout its use.

In some embodiments, the housing and/or housing lid can be made of a rigid material (e.g., plastic/polymer, metal, ceramic). In some embodiments, the housing and/or housing lid can be 3D-printed. In some embodiments, the internal dimensions of dampener 100 can be 90×80×10 mm and can be designed to fit a 50 mL FLEXBOY® bioprocessing bag. This dampener can be a single-use assembly for fluid flow systems which can absorb occurring pulsation by means of a gaseous cushion to reduce fluctuations in flow rate to a minimum. In some embodiments, the dead volume can depend on relative position in height of the bioprocessing bag in the housing. Thus, the height of the dampener can be defined to ensure constant dead volume and/or optimal dampening effect relative to requirements of the fluid path (e.g., flow rate and flow velocity, magnitude of the pulsation created by the pump head, system backpressure, etc.).

The pulsation dampener can include two fluids: (1) the displaced, pulsating fluid (e.g., water); and (2) a compressible fluid (e.g., air). When the displaced fluid is pumped through the bioprocessing bag, the trapped compressible fluid can absorb the pulsation within the displaced fluid and can pump on only a steady, pulsation free flow.

The pulsation dampening abilities of dampener disclosed in this section were systematically tested at different flow rates using a peristaltic pump with and without the dampener and a reference setup using a syringe pump. As explained in more detail below, it was shown that the dampener significantly decreases pulsation to an even lower level achieved with the syringe pump system, which was assumed to be pulsation free.

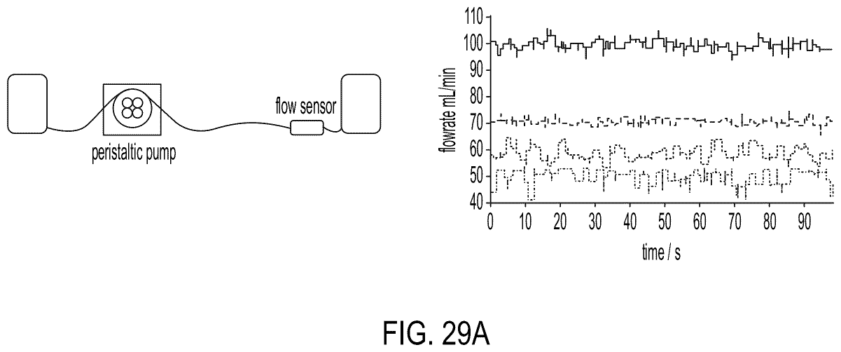

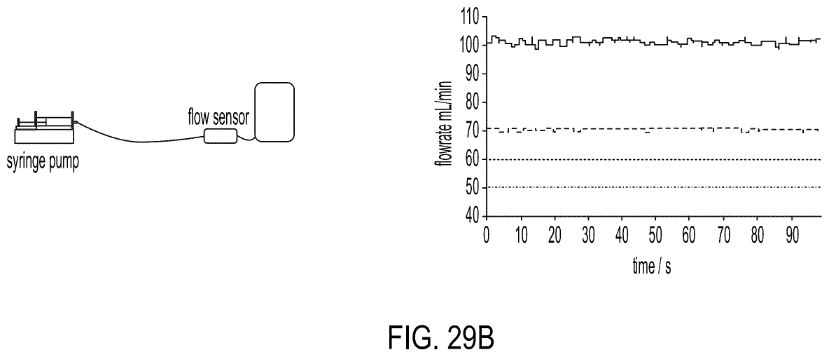

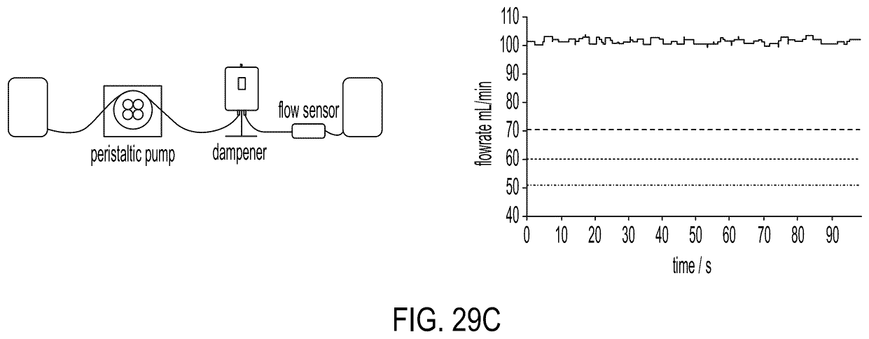

FIG. 29A illustrates a setup of a peristaltic pump using tubing with an inner diameter of 3.6 mm. Pulsation was measured with an ultrasonic flow sensor (Levitronix) with a time resolution of 0.1 seconds and a value resolution of 0.8 mL/min. Water was pumped through the system. FIG. 29A also shows the flow profile of the tested system over a total time period of 100 seconds. The system consisted of a peristaltic pump and the flow sensor. The tested flow rates were 50, 60, 70, and 100 mL/min. A high pulsation was observed due to the rolls of the peristaltic pump.

FIG. 29B illustrates a setup that consists of a syringe pump using tubing with an inner diameter of 3.6 mm. Pulsation was measured with the ultrasonic flow sensor with a time resolution of 0.1 seconds and a value resolution of 0.8 mL/min. Water was pumped through the system. FIG. 29B also shows the flow profile of the tested system over a total time period of 100 seconds. The tested flow rates were 50, 60, 70, and 100 mL/min and a low pulsation was measured.

FIG. 29C illustrates a setup that consists of a peristaltic pump using tubing with an inner diameter of 3.6 mm and a HPPD dampener as disclosed herein connected to that. The outlet of the HPPD dampener was connected with a check valve which had a breakthrough resistance of 0.14 bar. Pulsation was measure with the ultrasonic flow sensor with a time resolution of 0.1 seconds and a value resolution of 0.8 mL/min. Water was pumped through the system. FIG. 29C also shows the flow profile with the HPPD dampener. As shown, there was a significant decreased pulsation (lower amplitude). The HPPD effect was successfully (numerically) observed. In addition, the HPPD setup produced even less fluctuation in flow rate than the syringe pump.

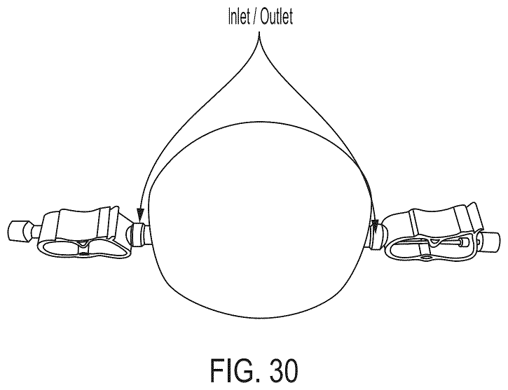

In order to test this rigid dampener, a comparability study was carried out between the HPPD dampener disclosed in this section and a Cole-Parmer HPPD, as shown in FIG. 30. The dampening principle of the Cole-Parmer HPPD is based on compression of a trapped air pocket. Specifically, the internal volume of the Cole-Parmer HPPD is approximately 190 mL. The dead volume while pumping is approximately 40-50 mL. This is a higher dead volume when compared to the HPPD dampener disclosed in this section while using the 50 mL bioprocessing bag. In addition, the Cole-Parmer HPPD takes longer to prime and must be placed in a fixed horizontal position to create the pulsation dampening effect. In contrast, the HPPD device disclosed in this section does not need to be horizontal but can be oriented in any direction during use. As explained in more detail below, the HPPD dampener described in this section has a much lower and more predictable dead volume and reduced priming time while showing even higher dampening efficiency at various flow rates when compared to the Cole-Parmer HPPD.

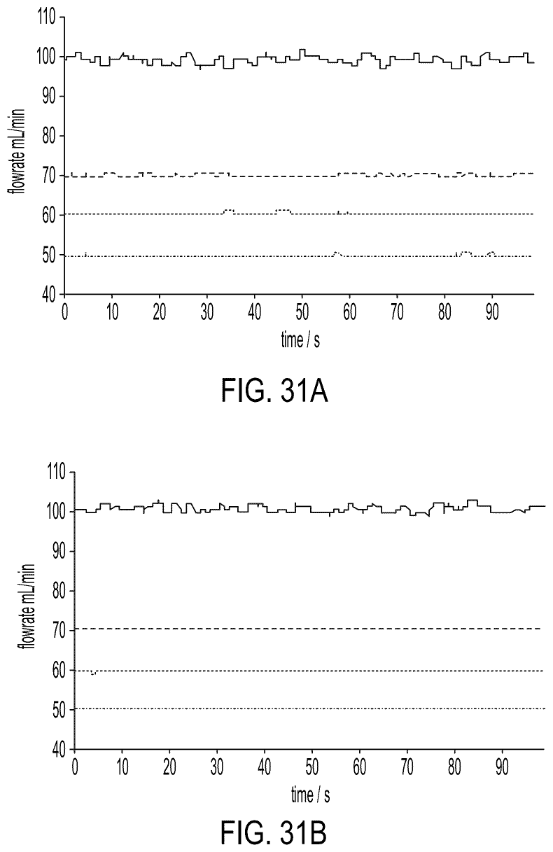

FIG. 31A illustrates the flow profile with the Cole-Parmer HPPD over a total time period of 100 seconds. The system consists of a peristaltic pump, the Cole-Parmer HPPD, and a flow sensor. Water was pumped at testing flow rates of 50, 60, 70, and 100 mL/min. The water was pumped with a peristaltic pump through a tubing with an inner diameter of 3.6 mm. Pulsation was measured with an ultrasonic flow sensor with a time resolution of 0.1 seconds and a value resolution of 0.8 mL/min.

FIG. 31B illustrates the flow profiles of the tested system (FIG. 29C) over a total period of 100 seconds. Water was tested at flow rates at 50, 60, 70, and 100 mL/min. Water was pumped with a peristaltic pump through a tubing with inner diameter of 3.6 mm. The outlet of the HPPD was connected with a check valve which had a breakthrough resistance of 0.14 bar. Again, pulsation was measured with the ultrasonic flow sensor with a resolution of 0.1 seconds and a value resolution of 0.8 mL/min. No significant difference of pulsation dampening between Cole-Parmer and the HPPD disclosed in this section were found.

Next, the filling volume and pressure was recorded as a function of the pumping flow rate after priming when the system has reached its operating equilibrium. It was shown that the dead volume of the HPPD dampener disclosed herein can stay constant after reaching a flow rate of approximately 70 mL/min and higher. To test, water was pumped with a peristaltic pump connected to the HPPD dampener disclosed in this section. A pressure sensor was installed on the inlet of the HPPD dampener and on the outlet with a check valve with 0.14 bar resistance. The flow rate was measured with an ultrasonic flow sensor with a time resolution of 0.1 seconds and a value resolution of 0.8 mL/min.

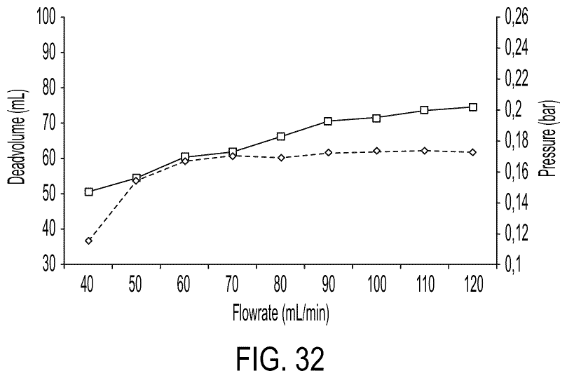

FIG. 32 illustrates the dead volume of the HPPD dampener disclosed in this section and the pressure at dampener inlet with increasing flow rate. Specifically, the pressure at the inlet of the dampener proportionally increases with the flow rate. The dead volume stabilizes at a flow rate of 70 mL/min. It can be decreased by reducing the total volume of the bioprocessing bag. However, the rigid housing played a big part of achieving the stable dampening effect. Additional parameter that might influence the dead volume and pressure can be changes in the hydrodynamic pressure, which can be adjusted by the relative positions of the pump, the dampener, and the fluid containers. It can influence the pressure equilibrium in the dampener and so the dead volume in the dampener. This effect may be negligible at higher flow rates (e.g., 200 mL/min). Another parameter can be the volume of the empty tubing between origin-fluid-container, the pump, and the dampener. The amount of air inside the dampener can be determined by the volume of the air in the empty tubing which is pumped into the dampener while the initial filling step of the system occurs. In FIG. 32, the diamond shaped icons represent the values of the dead volume and the squares represent the measured pressure.

Next, the influence of tubing diameter and length on pulsation was studied. The dampening effect is obtained by the absorption of the pulsation by the elasticity of the silicone tubing. Three different tubing diameters were investigated at a constant flow rate using a peristaltic pump. It was shown that with decreasing tubing diameter, the measured pulsation was significantly reduced. To measure, water was pumped with a peristaltic pump (model Watson Marlow 323) connected to different inner diameter tubing with a constant length of 1 meter. With increased tubing inner diameter, pumping RPM was decreased in order to achieve constant flux. Pulsation was measured with a MASTERFLEX® ultrasonic flow sensor with a measuring interval of 20 ms. For analysis, 300 measure points were analyzed per setup and arithmetic mean and standard deviation were calculated. Reduction in the flow rate standard deviation indicates improved pulsation dampening.

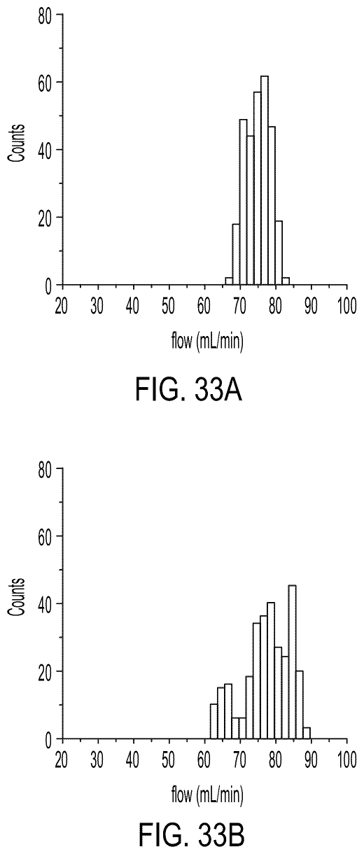

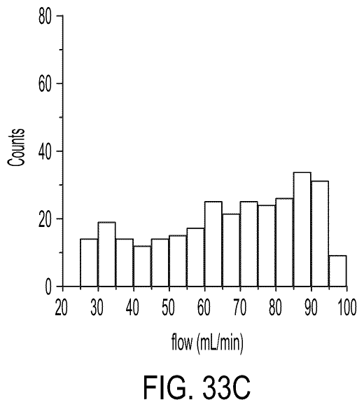

FIG. 33A illustrates the investigation of the pulsation for tubing with an inner diameter of 1.6 mm and fixed length of 1 meter with a pump speed of 285 rpm. Arithmetic mean is 75.6 mL/min and standard deviation is 3.4 mL/min. FIG. 33B illustrates the investigation of the pulsation for tubing with an inner diameter of 3.2 mm and fixed length of 1 meter with a pump speed of 70 rpm. Arithmetic mean is 77.3 mL/min and standard deviation is 6.7 mL/min. FIG. 33C illustrates the investigation of the pulsation for tubing with an inner diameter of 6 mm and fixed length of 1 meter with pump speed of 20 rpm. Arithmetic mean is 77.3 mL/min and standard deviation is 6.7 mL/min. With use of smaller tubing inner diameter and respective higher pumping speeds, flow rate standard deviation was significantly decreased and, at the same time, pulsation reduced.

Next, the pulsation dampening effect of increasing tubing length was investigated. This could demonstrate in addition to the reduction of the tubing diameter, increasing the tubing length can further lower pulsation. However, this leads to increased pressure loss. To test, water was pumped with a peristaltic pump connected to different tubing length and a constant inner diameter of 1.6 mm. Due to high back pressure, pumping RPM was increased with a tubing length of 20 meters in order to reach a flow rate comparable to the other setups. Pulsation was measured with the ultrasonic flow sensor with a measuring interval of 20 ms. For analysis, 300 measurement points were analyzed per setup and arithmetic mean and standard deviation were calculated. Reduction in flow rate standard deviation indicates improved pulsation dampening.

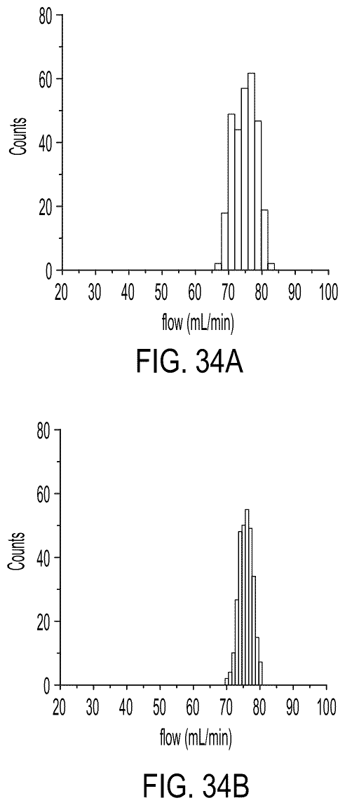

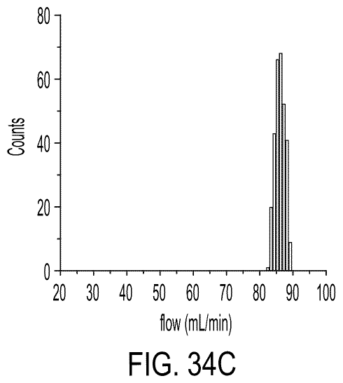

FIG. 34A illustrates the investigation of the pulsation for tubing with fixed length of 1 meter, an inner diameter of 1.6 mm, and a pump speed of 285 rpm. Arithmetic mean is mL/min and standard deviation is 3.4 mL/min. FIG. 34B illustrates the investigation of the pulsation for tubing with fixed length of 2 meters, an inner diameter of 1.6 mm, pump speed of 285 rpm. Arithmetic mean is 77.7 mL/min and standard deviation is 2.0 mL/min. FIG. 34C illustrates the investigation of the pulsation for tubing with fixed length of 20 meters, an inner diameter of 1.6 mm, and pump speed of 400 rpm. Arithmetic mean is 85.8 mL/min and standard deviation is 1.6 mL/min. With use of longer tubing and respective higher pumping speeds, flow rate standard deviation can significantly decrease and pulsation reduced. Compared to utilizing an HPPD dampener disclosed herein, the dead volume may be further decreased and a separate priming step could be skipped when using only tubing for pulsation dampening. However, pressure losses across the extended length of the tubing was much greater than it was when utilizing the HPPD dampener.

Materials used for the above experiments are in the following Table:

Material

Peristaltic pump; ISMATEC IP 65, MCP-process, Model: ISM915A
Syringe pump; kd Scientific, Model: Legato210
Tubing 1.6 mm; silicone tubing 1.6 × 4.8
Tubing 3.2 mm; silicone tubing 3.2 × 4.8
Tubing 6 mm; silicone tubing
Double check valve; DCV 125-001
Ultrasonic flow sensor; Levitronix
Water B.Braun
Pressure sensor; SciLog; SciPress Pressure; Flow Cell S1-240141-1019
Glass bottle; Schott
Flexboy; 50 mL bag
Cole parmer dampener; Masterflex; number: GZ-07596-20

### V. RNA Vaccines

Certain aspects of the present disclosure relate to the production, mixing or manufacture of pharmaceutical compositions comprising a personalized cancer vaccine (PCV). In some embodiments, the PCV is an RNA vaccine, including e.g, mRNA vaccines. Features of exemplary RNA vaccines are described infra. In some embodiments, the present disclosure provides an RNA polynucleotide or RNA molecule comprising one or more of the features/sequences of the RNA vaccines described infra. In some embodiments, the RNA polynucleotide or RNA molecule is a single-stranded mRNA polynucleotide. In other embodiments, the present disclosure provides a DNA polynucleotide encoding an RNA molecule comprising one or more of the features/sequences of the RNA vaccines described infra.

Personalized cancer vaccines comprise individualized neoantigens (i.e., tumor-associated antigens (TAAs) that are specifically expressed in the patient's cancer) identified as having potential immunostimulatory activities. In the embodiments described herein, the PCV is a nucleic acid, e.g., messenger RNA. Accordingly, without wishing to be bound by theory, it is believed that upon administration, the personalized cancer vaccine is taken up and translated by antigen presenting cells (APCs) and the expressed protein is presented via major histocompatibility complex (MHC) molecules on the surface of the APCs. This leads to an induction of both cytotoxic T-lymphocyte (CTL)-and memory T-cell-dependent immune responses against cancer cells expressing the TAA(s).

PCVs typically include multiple neoantigen epitopes (“neoepitopes”), e.g., 2, 3, 4, 6, 7, 8, 9, 10, 11, 12, 13, 14, 15, 16, 17, 18, 19, 20, 21, 22, 23, 24, 25, 26, 27, 28, 28, 29, or neoepitopes, or at least 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 neoepitopes, optionally with linker sequences between the individual neoepitopes. In some embodiments, a neoepitope as used herein refers to a novel epitope that is specific for a patient's cancer but not found in normal cells of the patient. In some embodiments, the neoepitope is presented to T cells when bound to MHC. In some embodiments, the PCV also includes a 5′ mRNA cap analogue, a 5′ UTR, a signal sequence, a domain to facilitate antigen expression, a 3′ UTR, and/or a polyA tail. In some embodiments, the RNA vaccine or RNA molecule which can be used with the methods and systems of the present disclosure comprises one or more polynucleotides encoding 10-20 neoepitopes resulting from cancer-specific somatic mutations present in the tumor specimen. In some embodiments, the RNA vaccine or RNA molecule comprises one or more polynucleotides encoding at least 5 neoepitopes resulting from cancer-specific somatic mutations present in the tumor specimen. In some embodiments, the RNA vaccine or RNA molecule comprises one or more polynucleotides encoding 5-20 neoepitopes resulting from cancer-specific somatic mutations present in the tumor specimen. In some embodiments, the RNA vaccine or RNA molecule comprises one or more polynucleotides encoding 5-10 neoepitopes resulting from cancer-specific somatic mutations present in the tumor specimen.

In some embodiments, the RNA vaccine or RNA molecule which can be used with the methods and systems of the present disclosure comprises one or more polynucleotide sequences encoding an amino acid linker. For example, amino acid linkers can be used between 2 patient-specific neoepitope sequences, between a patient-specific neoepitope sequence and a fusion protein tag (e.g., comprising sequence derived from an MHC complex polypeptide), or between a secretory signal peptide and a patient-specific neoepitope sequence. In some embodiments, the RNA vaccine or RNA molecule encodes multiple linkers. In some embodiments, the RNA vaccine or RNA molecule comprises one or more polynucleotides encoding 5-20 neoepitopes resulting from cancer-specific somatic mutations present in the tumor specimen, and the polynucleotides encoding each epitope are separated by a polynucleotide encoding a linker sequence. In some embodiments, the RNA vaccine or RNA molecule comprises one or more polynucleotides encoding 5-10 neoepitopes resulting from cancer-specific somatic mutations present in the tumor specimen, and the polynucleotides encoding each epitope are separated by a polynucleotide encoding a linker sequence. In some embodiments, polynucleotides encoding linker sequences are also present between the polynucleotides encoding an N-terminal fusion tag (e.g., a secretory signal peptide) and a polynucleotide encoding one of the neoepitopes and/or between a polynucleotide encoding one of the neoepitopes and the polynucleotides encoding a C-terminal fusion tag (e.g., comprising a portion of an MHC polypeptide). In some embodiments, two or more linkers encoded by the RNA vaccine or RNA molecule comprise different sequences. In some embodiments, the RNA vaccine or RNA molecule encodes multiple linkers, all of which share the same amino acid sequence.

A variety of linker sequences are known in the art. In some embodiments, the linker is a flexible linker. In some embodiments, the linker comprises G, S, A, and/or T residues. In some embodiments, the linker consists of glycine and serine residues. In some embodiments, the linker is between about 5 and about 20 amino acids or between about 5 and about 12 amino acids in length, e.g., about 5, about 6, about 7, about 8, about 9, about 10, about 11, about 12, about 13, about 14, about 15, about 16, about 17, about 18, about 19, or about 20 amino acids in length. In some embodiments, the linker comprises the sequence GGSGGGGSGG (SEQ ID NO:21). In some embodiments, the linker of the RNA vaccine or RNA molecule comprises the sequence GGCGGCUCUGGAGGAGGCGGCUCCGGAGGC (SEQ ID NO:19). In some embodiments, the linker of the RNA vaccine or RNA molecule is encoded by DNA comprising the sequence GGCGGCTCTGGAGGAGGCGGCTCCGGAGGC (SEQ ID NO:20).

In some embodiments, the RNA vaccine or RNA molecule which can be used with the methods and systems of the present disclosure comprises a 5′ cap. The basic mRNA cap structure is known to contain a 5′-5′ triphosphate linkage between 2 nucleosides (e.g., two guanines) and a 7-methyl group on the distal guanine, i.e., m⁷ GpppG. Exemplary cap structures can be found, e.g., in U.S. Pat. Nos. 8,153,773 and 9,295,717 and Kuhn, A. N. et al. (2010) Gene Ther. 17:961-971. In some embodiments, the 5′ cap has the structure m₂^{7,2′-O}GppₛpG. In some embodiments, the 5′ cap is a beta-S-ARCA cap. The S-ARCA cap structure includes a 2′-O methyl substitution (e.g., at the C2′ position of the m⁷G) and an S-substitution at one or more of the phosphate groups. In some embodiments, the 5′ cap comprises the structure:

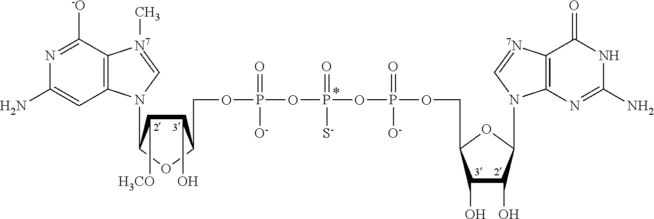

In some embodiments, the 5′ cap is the D1 diastereoisomer of beta-S-ARCA (see, e.g., U.S. Pat. No. 9,295,717). The * in the above structure indicates a stereogenic P center, which can exist in two diastereoisomers (designated D1 and D2). The D1 diastereomer of beta-S-ARCA or beta-S-ARCA(D1) is the diastereomer of beta-S-ARCA which elutes first on an HPLC column compared to the D2 diastereomer of beta-S-ARCA (beta-S-ARCA(D2)) and thus exhibits a shorter retention time. The HPLC preferably is an analytical HPLC. In one embodiment, a Supelcosil LC-18-T RP column, preferably of the format: 5 μm, 4.6×250 mm is used for separation, whereby a flow rate of 1.3 ml/min can be applied. In one embodiment, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

In some embodiments, the RNA vaccine or RNA molecule which can be used with the methods and systems of the present disclosure comprises a 5′ UTR. Certain untranslated sequences found 5′ to protein-coding sequences in mRNAs have been shown to increase translational efficiency. See, e.g., Kozak, M. (1987) J. Mol. Biol. 196:947-950. In some embodiments, the 5′ UTR comprises sequence from the human alpha globin mRNA. In some embodiments, the RNA vaccine or RNA molecule comprises a 5′ UTR sequence of UUCUUCUGGUCCCCACAGACUCAGAGAGAACCCGCCACC (SEQ ID NO:5). In some embodiments, the 5′ UTR sequence of the RNA vaccine or RNA molecule is encoded by DNA comprising the sequence TTCTTCTGGTCCCCACAGACTCAGAGAGAACCCGCCACC (SEQ ID NO:6). In some embodiments, the 5′ UTR sequence of RNA vaccine or RNA molecule comprises the sequence

(SEQ ID NO: 3)
GGCGAACUAGUAUUCUUCUGGUCCCCACAGACUCAGAGAGAACCCGCC
ACC.

In some embodiments, the 5′ UTR sequence of RNA vaccine or RNA molecule is encoded by DNA comprising the sequence

(SEQ ID NO: 4)
GGCGAACTAGTATTCTTCTGGTCCCCACAGACTCAGAGAGAACCCGCC
ACC.

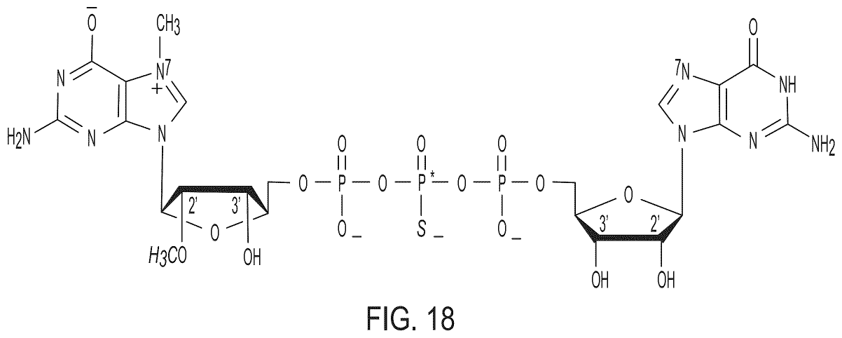

In some embodiments of the methods provided herein, the constant region of an exemplary RNA vaccine comprises the ribonucleotide sequence (5′->3′) of SEQ ID NO: 24. The linkage between the first two G residues is the unusual bond (5′→5′)-ppₛp-, e.g., as shown in Table 6 and in FIG. 18 for the 5′ capping structure. “N” refers to the position of polynucleotide sequence(s) encoding one or more (e.g., 1-20) neoepitopes (separated by optional linkers). The insertion site for tumor-specific sequences (C131-A132; marked in bold text) is depicted in bold text. See Table 6 for the modified bases and uncommon links in the exemplary RNA sequence.

TABLE 6

| Type | Location | Description |
|---|---|---|
| Modified Base | G1 | m₂^{7•2′•O} G |
| Uncommon | G1-G2 | (5′→5′)-ppₛp- |
| Link | | |
| Uncommon | C131-A132 | Insertion site for tumor-specific |
| Link | | sequences |

In some embodiments, the RNA vaccine or RNA molecule which can be used with the methods and systems of the present disclosure comprises a polynucleotide sequence encoding a secretory signal peptide. As is known in the art, a secretory signal peptide is an amino acid sequence that directs a polypeptide to be trafficked from the endoplasmic reticulum and into the secretory pathway upon translation. In some embodiments, the signal peptide is derived from a human polypeptide, such as an MHC polypeptide. See, e.g., Kreiter, S. et al. (2008) J. Immunol. 180:309-318, which describes an exemplary secretory signal peptide that improves processing and presentation of MHC Class I and II epitopes in human dendritic cells. In some embodiments, upon translation, the signal peptide is N-terminal to one or more neoepitope sequence(s) encoded by the RNA vaccine. In some embodiments, the secretory signal peptide comprises the sequence MRVMAPRTLILLLSGALALTETWAGS (SEQ ID NO:9). In some embodiments, the secretory signal peptide of the RNA vaccine or RNA molecule comprises the sequence AUGAGAGUGAUGGCCCCCAGAACCCUGAUCCUGCUGCUGUCUGGCGCCCUGGC CCUGACAGAGACAUGGGCCGGAAGC (SEQ ID NO:7). In some embodiments, the secretory signal peptide of the RNA vaccine or RNA molecule is encoded by DNA comprising the sequence

(SEQ ID NO: 8)
ATGAGAGTGATGGCCCCCAGAACCCTGATCCTGCTGCTGTCTGGCGCC
CTGGCCCTGACAGAGACATGGGCCGGAAGC.

In some embodiments, the RNA vaccine or RNA molecule which can be used with the methods and systems of the present disclosure comprises a polynucleotide sequence encoding at least a portion of a transmembrane and/or cytoplasmic domain. In some embodiments, the transmembrane and/or cytoplasmic domains are from the transmembrane/cytoplasmic domains of an MHC molecule. The term “major histocompatibility complex” and the abbreviation “MHC” relate to a complex of genes which occurs in all vertebrates. The function of MHC proteins or molecules in signaling between lymphocytes and antigen-presenting cells in normal immune responses involves them binding peptides and presenting them for possible recognition by T-cell receptors (TCR). MHC molecules bind peptides in an intracellular processing compartment and present these peptides on the surface of antigen-presenting cells to T cells. The human MHC region, also referred to as HLA, is located on chromosome 6 and comprises the class I region and the class II region. The class I alpha chains are glycoproteins having a molecular weight of about 44 kDa. The polypeptide chain has a length of somewhat more than 350 amino acid residues. It can be divided into three functional regions: an external, a transmembrane and a cytoplasmic region. The external region has a length of 283 amino acid residues and is divided into three domains, alpha1, alpha2 and alpha3. The domains and regions are usually encoded by separate exons of the class I gene. The transmembrane region spans the lipid bilayer of the plasma membrane. It consists of 23 usually hydrophobic amino acid residues which are arranged in an alpha helix. The cytoplasmic region, i.e. the part which faces the cytoplasm and which is connected to the transmembrane region, typically has a length of 32 amino acid residues and is able to interact with the elements of the cytoskeleton. The alpha chain interacts with beta2-microglobulin and thus forms alpha-beta2 dimers on the cell surface. The term “MHC class II” or “class II” relates to the major histocompatibility complex class II proteins or genes. Within the human MHC class II region there are the DP, DQ and DR subregions for class II alpha chain genes and beta chain genes (i.e. DPalpha, DPbeta, DQalpha, DQbeta, DRalpha and DRbeta). Class II molecules are heterodimers each consisting of an alpha chain and a beta chain. Both chains are glycoproteins having a molecular weight of 31-34 kDa (a) or 26-29 kDA (beta). The total length of the alpha chains varies from 229 to 233 amino acid residues, and that of the beta chains from 225 to 238 residues. Both alpha and beta chains consist of an external region, a connecting peptide, a transmembrane region and a cytoplasmic tail. The external region consists of two domains, alpha1 and alpha2 or beta1 and beta2. The connecting peptide is respectively beta and 9 residues long in alpha and beta chains. It connects the two domains to the transmembrane region which consists of 23 amino acid residues both in alpha chains and in beta chains. The length of the cytoplasmic region, i.e. the part which faces the cytoplasm and which is connected to the transmembrane region, varies from 3 to 16 residues in alpha chains and from 8 to 20 residues in beta chains. Exemplary transmembrane/cytoplasmic domain sequences are described in U.S. Pat. Nos. 8,178,653 and 8,637,006. In some embodiments, upon translation, the transmembrane and/or cytoplasmic domain is C-terminal to one or more neoepitope sequence(s) encoded by the RNA vaccine. In some embodiments, the transmembrane and/or cytoplasmic domain of the MHC molecule comprises the sequence

(SEQ ID NO: 12)
IVGIVAGLAVLAVVVIGAVVATVMCRRKSSGGKGGSYSQAASSDSAQG
SDVSLTA.

In some embodiments, the transmembrane and/or cytoplasmic domain of the MHC molecule comprises the sequence AUCGUGGGAAUUGUGGCAGGACUGGCAGUGCUGGCCGUGGUGGUGAUCGGAG CCGUGGUGGCUACCGUGAUGUGCAGACGGAAGUCCAGCGGAGGCAAGGGCGGC AGCUACAGCCAGGCCGCCAGCUCUGAUAGCGCCCAGGGCAGCGACGUGUCACU GACAGCC (SEQ ID NO:10). In some embodiments, the transmembrane and/or cytoplasmic domain of the MHC molecule is encoded by DNA comprising the sequence

(SEQ ID NO: 11)
ATCGTGGGAATTGTGGCAGGACTGGCAGTGCTGGCCGTGGTGGTGATC
GGAGCCGTGGTGGCTACCGTGATGTGCAGACGGAAGTCCAGCGGAGGC
AAGGGCGGCAGCTACAGCCAGGCCGCCAGCTCTGATAGCGCCCAGGGC
AGCGACGTGTCACTGACAGCC.

In some embodiments, the RNA vaccine or RNA molecule which can be used with the methods and systems of the present disclosure comprises both a polynucleotide sequence encoding a secretory signal peptide that is N-terminal to the one or more neoepitope sequence(s) and a polynucleotide sequence encoding a transmembrane and/or cytoplasmic domain that is C-terminal to the one or more neoepitope sequence(s). Combining such sequences has been shown to improve processing and presentation of MHC Class I and II epitopes in human dendritic cells. See, e.g., Kreiter, S. et al. (2008) J. Immunol. 180:309-318.

In myeloid DCs, the RNA is released into the cytosol and translated into a poly-neoepitopic peptide. The polypeptide contains additional sequences to enhance antigen presentation. In some embodiments, a signal sequence (sec) from the MHCI heavy chain at the N-terminal of the polypeptide is used to target the nascent molecule to the endoplasmic reticulum, which has been shown to enhance MHCI presentation efficiency. Without wishing to be bound by theory, it is believed that the transmembrane and cytoplasmic domains of MHCI heavy chain guide the polypeptide to the endosomal/lysosomal compartments that were shown to improve MHCII presentation.

In some embodiments, the RNA vaccine or RNA molecule which can be used with the methods and systems of the present disclosure comprises a 3′UTR. Certain untranslated sequences found 3′ to protein-coding sequences in mRNAs have been shown to improve RNA stability, translation, and protein expression. Polynucleotide sequences suitable for use as 3′ UTRs are described, for example, in PG Pub. No. US20190071682. In some embodiments, the 3′ UTR comprises the 3′ untranslated region of AES or a fragment thereof and/or the non-coding RNA of the mitochondrially encoded 12S RNA. The term “AES” relates to Amino- Terminal Enhancer Of Split and includes the AES gene (see, e.g., NCBI Gene ID:166). The protein encoded by this gene belongs to the groucho/TLE family of proteins, can function as a homooligomer or as a heteroologimer with other family members to dominantly repress the expression of other family member genes. An exemplary AES mRNA sequence is provided in NCBI Ref. Seq. Accession NO. NM_198969. The term “MT_RNR1” relates to Mitochondrially Encoded 12S RNA and includes the MT_RNR1 gene (see, e.g., NCBI Gene ID:4549). This RNA gene belongs to the Mt_rRNA class. Diseases associated with MT-RNR1 include restrictive cardiomyopathy and auditory neuropathy. Among its related pathways are Ribosome biogenesis in eukaryotes and CFTR translational fidelity (class I mutations). An exemplary MT_RNR1 RNA sequence is present within the sequence of NCBI Ref. Seq. Accession NO. NC_012920. In some embodiments, the 3′ UTR of the RNA vaccine or RNA molecule comprises the sequence CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACU CACCACCUCUGCUAGUUCCAGACACCUCC (SEQ ID NO:15). In some embodiments, the 3′ UTR of the RNA vaccine or RNA molecule comprises the sequence CAAGCACGCAGCAAUGCAGCUCAAAACGCUUAGCCUAGCCACACCCCCACGGG AAACAGCAGUGAUUAACCUUUAGCAAUAAACGAAAGUUUAACUAAGCUAUAC UAACCCCAGGGUUGGUCAAUUUCGUGCCAGCCACACCG (SEQ ID NO:17). In some embodiments, the 3′ UTR of the RNA vaccine or RNA molecule comprises the sequence CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACU CACCACCUCUGCUAGUUCCAGACACCUCC (SEQ ID NO:15) and the sequence CAAGCACGCAGCAAUGCAGCUCAAAACGCUUAGCCUAGCCACACCCCCACGGG AAACAGCAGUGAUUAACCUUUAGCAAUAAACGAAAGUUUAACUAAGCUAUAC UAACCCCAGGGUUGGUCAAUUUCGUGCCAGCCACACCG (SEQ ID NO:17). In some embodiments, the 3′ UTR of the RNA vaccine or RNA molecule comprises the sequence CUCGAGCUGGUACUGCAUGCACGCAAUGCUAGCUGCCCCUUUCCCGUCCUGGG UACCCCGAGUCUCCCCCGACCUCGGGUCCCAGGUAUGCUCCCACCUCCACCUGC CCCACUCACCACCUCUGCUAGUUCCAGACACCUCCCAAGCACGCAGCAAUGCA GCUCAAAACGCUUAGCCUAGCCACACCCCCACGGGAAACAGCAGUGAUUAACC UUUAGCAAUAAACGAAAGUUUAACUAAGCUAUACUAACCCCAGGGUUGGUCA AUUUCGUGCCAGCCACACCGAGACCUGGUCCAGAGUCGCUAGCCGCGUCGCU (SEQ ID NO:13). In some embodiments, the 3′ UTR of the RNA vaccine or RNA molecule is encoded by DNA comprising the sequence CTGGTACTGCATGCACGCAATGCTAGCTGCCCCTTTCCCGTCCTGGGTACCCCGA GTCTCCCCCGACCTCGGGTCCCAGGTATGCTCCCACCTCCACCTGCCCCACTCAC CACCTCTGCTAGTTCCAGACACCTCC (SEQ ID NO:16). In some embodiments, the 3′ UTR of the RNA vaccine or RNA molecule is encoded by DNA comprising the sequence CAAGCACGCAGCAATGCAGCTCAAAACGCTTAGCCTAGCCACACCCCCACGGGA AACAGCAGTGATTAACCTTTAGCAATAAACGAAAGTTTAACTAAGCTATACTAA CCCCAGGGTTGGTCAATTTCGTGCCAGCCACACCG (SEQ ID NO:18). In some embodiments, the 3′ UTR of the RNA vaccine or RNA molecule is encoded by DNA comprising the sequence CTGGTACTGCATGCACGCAATGCTAGCTGCCCCTTTCCCGTCCTGGGTACCCCGA GTCTCCCCCGACCTCGGGTCCCAGGTATGCTCCCACCTCCACCTGCCCCACTCAC CACCTCTGCTAGTTCCAGACACCTCC (SEQ ID NO:16) and the sequence CAAGCACGCAGCAATGCAGCTCAAAACGCTTAGCCTAGCCACACCCCCACGGGA AACAGCAGTGATTAACCTTTAGCAATAAACGAAAGTTTAACTAAGCTATACTAA CCCCAGGGTTGGTCAATTTCGTGCCAGCCACACCG (SEQ ID NO:18). In some embodiments, the 3′ UTR of the RNA vaccine or RNA molecule is encoded by DNA comprising the sequence

(SEQ ID NO: 14)
CTGGTACTGCATGCACGCAATGCTAGCTGCCCCTTTCCCGTCCTGGGT
ACCCCGAGTCTCCCCCGACCTCGGGTCCCAGGTATGCTCCCACCTCCA
CCTGCCCCACTCACCACCTCTGCTAGTTCCAGACACCTCCCAAGCACG
CAGCAATGCAGCTCAAAACGCTTAGCCTAGCCACACCCCCACGGGAAA
CAGCAGTGATTAACCTTTAGCAATAAACGAAAGTTTAACTAAGCTATA
CTAACCCCAGGGTTGGTCAATTTCGTGCCAGCCACACCGAGACCTGGT
CCAGAGTCGCTAGCCGCGTCGCT.

In some embodiments, the which can be used with the methods and systems of the present disclosure comprises the general structure (in the 5′ 43′ direction): (1) a 5′ cap; (2) a 5′ untranslated region (UTR); (3) a polynucleotide sequence encoding a secretory signal peptide; (4) a polynucleotide sequence encoding at least a portion of a transmembrane and cytoplasmic domain of a major histocompatibility complex (MHC) molecule; (5) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (6) a poly(A) sequence. In some embodiments, an RNA vaccine or an RNA molecule which can be used with the methods and systems of the present disclosure comprises, in the 5′→3′ direction: the polynucleotide sequence

GGCGAACUAGUAUUCUUCUGGUCCCCACAGACUCAGAGAGAACCCGCCACCAU GAGAGUGAUGGCCCCCAGAACCCUGAUCCUGCUGCUGUCUGGCGCCCUGGCCC UGACAGAGACAUGGGCCGGAAGC (SEQ ID NO:1); and the polynucleotide sequence AUCGUGGGAAUUGUGGCAGGACUGGCAGUGCUGGCCGUGGUGGUGAUCGGAG CCGUGGUGGCUACCGUGAUGUGCAGACGGAAGUCCAGCGGAGGCAAGGGCGGC AGCUACAGCCAGGCCGCCAGCUCUGAUAGCGCCCAGGGCAGCGACGUGUCACU GACAGCCUAGUAACUCGAGCUGGUACUGCAUGCACGCAAUGCUAGCUGCCCCU UUCCCGUCCUGGGUACCCCGAGUCUCCCCCGACCUCGGGUCCCAGGUAUGCUC CCACCUCCACCUGCCCCACUCACCACCUCUGCUAGUUCCAGACACCUCCCAAGC ACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACC CCAGGGUUGGUCAAUUUCGUGCCAGCCACACCGAGACCUGGUCCAGAGUCGCU AGCCGCGUCGCU (SEQ ID NO:2). Advantageously, RNA vaccines comprising this combination and orientation of structures or sequences are characterized by one or more of: improved RNA stability, enhanced translational efficiency, improved antigen presentation and/or processing (e.g., by DCs), and increased protein expression.

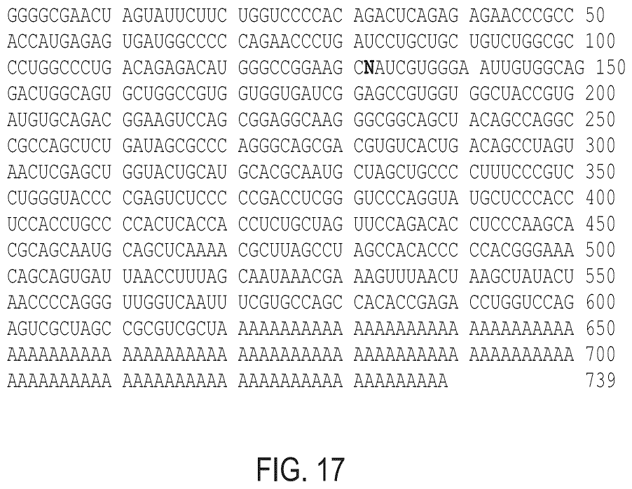

In some embodiments, an RNA vaccine or RNA molecule of the present disclosure comprises the sequence (in the 5′→3′ direction) of SEQ ID NO:24. See, e.g., FIG. 17. In some embodiments, N refers to a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different neoepitopes. In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules). In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules) and an additional amino acid linker at the 3′ end.

In some embodiments, the RNA vaccine or RNA molecule further comprises a polynucleotide sequence encoding at least one neoepitopes; wherein the polynucleotide sequence encoding the at least one neoepitope is between the polynucleotide sequence encoding the secretory signal peptide and the polynucleotide sequence encoding the at least portion of the transmembrane and cytoplasmic domain of the MHC molecule in the 5′ 43′ direction. In some embodiments, the RNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes.

In some embodiments, the RNA vaccine or an RNA molecule which can be used with the methods and systems of the present disclosure further comprises, in the 5′ 43′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′ 43′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the MHC molecule, or between the sequences of SEQ ID NO:1 and SEQ ID NO:2, in the 5′→3′ direction. In some embodiments, the RNA vaccine or molecule comprises 2, 3, 4, 5, 6, 7, 8, 9, 11, 12, 13, 14, 15, 16, 17, 18, 19, 20, 21, 22, 23, 24, 25, 26, 27, 28, 28, 29, or 30 linker-epitope modules. In some embodiments, each of the linker-epitope modules encodes a different neoepitope. In some embodiments, the RNA vaccine or molecule comprises 2, 3, 4, 5, 6, 7, 8, 9, 10, 11, 12, 13, 14, 15, 16, 17, 18, 19, or 20 linker-epitope modules, and the RNA vaccine or molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA vaccine or molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

In some embodiments, the RNA vaccine or an RNA molecule which can be used with the methods and systems of the present disclosure is at least 800 nucleotides, at least 1000 nucleotides, or at least 1200 nucleotides in length. In some embodiments, the RNA vaccine is less than 2000 nucleotides in length. In some embodiments, the RNA vaccine is at least 800 nucleotides but less than 2000 nucleotides in length, at least 1000 nucleotides but less than 2000 nucleotides in length, at least 1200 nucleotides but less than 2000 nucleotides in length, at least 1400 nucleotides but less than 2000 nucleotides in length, at least 800 nucleotides but less than 1400 nucleotides in length, or at least 800 nucleotides but less than 2000 nucleotides in length. For example, the constant regions of an RNA vaccine comprising the elements described above are approximately 800 nucleotides in length. In some embodiments, an RNA vaccine comprising 5 patient-specific neoepitopes (e.g., each encoding 27 amino acids) is greater than 1300 nucleotides in length. In some embodiments, an RNA vaccine comprising patient-specific neoepitopes (e.g., each encoding 27 amino acids) is greater than 1800 nucleotides in length.

### Lipoplexes/Liposomes

In some embodiments, the RNA vaccine or an RNA molecule which can be used with the methods and systems of the present disclosure is formulated in a lipoplex nanoparticle or liposome. In some embodiments, a lipoplex nanoparticle formulation for the RNA (RNA-Lipoplex) is used to enable IV delivery of an RNA vaccine of the present disclosure. In some embodiments, a lipoplex nanoparticle formulation for the RNA cancer vaccine comprising the synthetic cationic lipid (R)-N,N,N-trimethyl-2,3-dioleyloxy-1-propanaminium chloride (DOTMA) and the phospholipid 1,2-dioleoyl-sn-glycero-3-phosphoethanolamine (DOPE) is used, e.g., to enable IV delivery. The DOTMA/DOPE liposomal component has been optimized for IV delivery and targeting of antigen-presenting cells in the spleen and other lymphoid organs.

In some embodiments, RNA molecule which can be used with the methods and systems of the present disclosure is mixed with a pharmaceutical composition comprising one or more cationic lipids, including, e.g., (R)-N,N,N-trimethyl-2,3-dioleyloxy-1-propanaminium chloride (DOTMA) and the phospholipid 1,2-dioleoyl-sn-glycero-3-phosphoethanolamine (DOPE). In one embodiment, the pharmaceutical composition comprises at least one lipid. In one embodiment, the pharmaceutical composition comprises at least one cationic lipid. The cationic lipid can be monocationic or polycationic. Any cationic amphiphilic molecule, eg, a molecule which comprises at least one hydrophilic and lipophilic moiety is a cationic lipid within the meaning of the present invention. In one embodiment, the positive charges are contributed by the at least one cationic lipid and the negative charges are contributed by the RNA. In one embodiment, the pharmaceutical composition comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid.

In one embodiment, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof.

In one embodiment, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof.

In one embodiment, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In one embodiment, in this ratio, the molar amount of the cationic lipid results from the molar amount of the cationic lipid multiplied by the number of positive charges in the cationic lipid.

In one embodiment, the lipid is comprised in a vesicle encapsulating said RNA. The vesicle may be a multilamellar vesicle, an unilamellar vesicle, or a mixture thereof. The vesicle may be a liposome.

Lipoplexes or liposomes described herein can be formed by adjusting a positive to negative charge, depending on the (+/−) charge ratio of a cationic lipid to RNA and mixing the RNA and the cationic lipid. The +/− charge ratio of the cationic lipid to the RNA in the pharmaceutical compositions described herein can be calculated by the following equation. (+/− charge ratio)=[(cationic lipid amount (mol))*(the total number of positive charges in the cationic lipid)]:[(RNA amount (mol))*(the total number of negative charges in RNA)]. The RNA amount and the cationic lipid amount can be easily determined by one skilled in the art in view of a loading amount upon preparation of the nanoparticles. For further descriptions of exemplary pharmaceutical compositions, see, e.g., PG Pub. No. US20150086612.

In one embodiment, the overall charge ratio of positive charges to negative charges in the pharmaceutical compositions (e.g., at physiological pH) is between 1.4:1 and 1:8, preferably between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In some embodiments, at physiological pH the overall charge ratio of positive charges to negative charges of the nanoparticles is between 1:1.2 (0.83) and 1:2 (0.5). In some embodiments, at physiological pH the overall charge ratio of positive charges to negative charges of the pharmaceutical compositions is between 1.6:2 (0.8) and 1:2 (0.5) or between 1.6:2 (0.8) and 1.1:2 (0.55). In some embodiments, at physiological pH the overall charge ratio of positive charges to negative charges of the pharmaceutical composition is 1.3:2 (0.65). In some embodiments, at physiological pH the overall charge ratio of positive charges to negative charges of the liposome is not lower than 1.0:2.0. In some embodiments, at physiological pH the overall charge ratio of positive charges to negative charges of the liposome is not higher than 1.9:2.0. In some embodiments, at physiological pH the overall charge ratio of positive charges to negative charges of the liposome is not lower than 1.0:2.0 and not higher than 1.9:2.0. As is apparent to a skilled person, the pharmaceutical compositions of the present disclosure may comprise a first pharmaceutical composition comprising RNA and a second pharmaceutical composition comprising a lipid such than when mixed using the methods and systems of the present disclosure the aforementioned positive to negative charges are achieved.

In one embodiment, the pharmaceutical compositions comprise DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the pharmaceutical compositions comprise DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the pharmaceutical compositions comprise DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the pharmaceutical compositions comprising DOTMA and DOPE in a molar ratio of 2:1 to 1:2, preferably 2:1 to 1:1, and wherein the charge ratio of positive charges in DOTMA to negative charges in the RNA is 1.4:1 or less. In one embodiment, the pharmaceutical compositions comprise DOTMA and cholesterol in a molar ratio of 2:1 to 1:2, preferably 2:1 to 1:1, and wherein the charge ratio of positive charges in DOTMA to negative charges in the RNA is 1.4:1 or less. In one embodiment, the pharmaceutical compositions comprise DOTAP and DOPE in a molar ratio of 2:1 to 1:2, preferably 2:1 to 1:1, and wherein the charge ratio of positive charges in DOTAP to negative charges in the RNA is 1.4:1 or less. As is apparent to a skilled person, the pharmaceutical compositions of the present disclosure may comprise a first pharmaceutical composition comprising RNA and a second pharmaceutical composition comprising a lipid (including, e.g., DOTMA, DOPE, DOTAP and/or cholesterol) such than when mixed using the methods and systems of the present disclosure the aforementioned positive to negative charge ratios and/or molar ratios are achieved.

In one embodiment, the zeta potential of the lipoplexes or liposomes produced or manufactured after combining two or more pharmaceutical compositions described herein following the methods and systems of the disclosure is −5 or less, −10 or less, −15 or less, -20 or less or −25 or less. In various embodiments, the zeta potential of the lipoplexes or liposomes is −35 or higher, −30 or higher or −25 or higher. In one embodiment, the nanoparticles or liposomes have a zeta potential from 0 mV to −50 mV, preferably 0 mV to -40 mV or −10 mV to −30 mV.

In some embodiments, the polydispersity index of the lipoplexes or liposomes produced or manufactured after combining two or more pharmaceutical compositions described herein following the methods and systems of the disclosure is 0.5 or less, 0.4 or less, or 0.3 or less, as measured by dynamic light scattering.

In some embodiments, the nanoparticles or liposomes produced or manufactured after combining two or more pharmaceutical compositions described herein following the methods and systems of the disclosure have an average diameter in the range of about 50 nm to about 1000 nm, from about 100 nm to about 800 nm, from about 200 nm to about 600 nm, from about 250 nm to about 700 nm, or from about 250 nm to about 550 nm, as measured by dynamic light scattering.

Further provided herein are DNA molecules encoding any of the RNA vaccines or RNA molecules which can be used with the methods and systems of the present disclosure. For example, in some embodiments, a DNA molecule of the present disclosure comprises the general structure (in the 5′→3′ direction): (1) a polynucleotide sequence encoding a 5′ untranslated region (UTR); (2) a polynucleotide sequence encoding a secretory signal peptide; (3) a polynucleotide sequence encoding at least a portion of a transmembrane and cytoplasmic domain of a major histocompatibility complex (MHC) molecule; (4) a polynucleotide sequence encoding a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (5) a polynucleotide sequence encoding a poly(A) sequence. In some embodiments, a DNA molecule of the present disclosure comprises, in the 43′ direction: the polynucleotide sequence GGCGAACTAGTATTCTTCTGGTCCCCACAGACTCAGAGAGAACCCGCCACCATG AGAGTGATGGCCCCCAGAACCCTGATCCTGCTGCTGTCTGGCGCCCTGGCCCTGA CAGAGACATGGGCCGGAAGC (SEQ ID NO:22); and the polynucleotide sequence ATCGTGGGAATTGTGGCAGGACTGGCAGTGCTGGCCGTGGTGGTGATCGGAGCC GTGGTGGCTACCGTGATGTGCAGACGGAAGTCCAGCGGAGGCAAGGGCGGCAGC TACAGCCAGGCCGCCAGCTCTGATAGCGCCCAGGGCAGCGACGTGTCACTGACA GCCTAGTAACTCGAGCTGGTACTGCATGCACGCAATGCTAGCTGCCCCTTTCCCG TCCTGGGTACCCCGAGTCTCCCCCGACCTCGGGTCCCAGGTATGCTCCCACCTCC ACCTGCCCCACTCACCACCTCTGCTAGTTCCAGACACCTCCCAAGCACGCAGCAA TGCAGCTCAAAACGCTTAGCCTAGCCACACCCCCACGGGAAACAGCAGTGATTA ACCTTTAGCAATAAACGAAAGTTTAACTAAGCTATACTAACCCCAGGGTTGGTCA ATTTCGTGCCAGCCACACCGAGACCTGGTCCAGAGTCGCTAGCCGCGTCGCT (SEQ ID NO:23).

In some embodiments, the DNA molecule further comprises, in the 5′ 43′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′ 43′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the WIC molecule, or between the sequences of SEQ ID NO:22 and SEQ ID NO:23, in the 5′→3′ direction. In some embodiments, the DNA molecule comprises 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 linker-epitope modules, and each of the linker-epitope modules encodes a different neoepitope. In some embodiments, the DNA molecule comprises 2, 3, 4, 5, 6, 7, 8, 9, 10, 11, 12, 13, 14, 15, 16, 17, 18, 19, or 20 linker-epitope modules, and the DNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or different neoepitopes. In some embodiments, the DNA molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

In some embodiments, an RNA or DNA molecule of the present disclosure comprises a type IIS restriction cleavage site, which allows RNA to be transcribed under the control of a 5′ RNA polymerase promoter and which contains a polyadenyl cassette (poly(A) sequence), wherein the recognition sequence is located 3′ of the poly(A) sequence, while the cleavage site is located upstream and thus within the poly(A) sequence. Restriction cleavage at the type IIS restriction cleavage site enables a plasmid to be linearized within the poly(A) sequence, as described in U.S. Pat. Nos. 9,476,055 and 10,106,800. The linearized plasmid can then be used as template for in vitro transcription, the resulting transcript ending in an unmasked poly(A) sequence. Any of the type IIS restriction cleavage sites described in U.S. Pat. Nos. 9,476,055 and 10,106,800 may be used.

In some embodiments of the methods provided herein, the RNA vaccine or RNA molecule includes one or more polynucleotides encoding 10-20 (e.g., any of 10, 11, 12, 13, 14, 16, 17, 18, 19, or 20) neoepitopes resulting from cancer-specific somatic mutations present in the tumor specimen. In certain embodiments, the RNA vaccine or RNA molecule is formulated in a lipoplex nanoparticle or liposome. In certain embodiments, the lipoplex nanoparticle or liposome includes one or more lipids that form a multilamellar structure that encapsulates the RNA of the RNA vaccine. In certain embodiments, the one or more lipids include at least one cationic lipid and at least one helper lipid. In certain embodiments, the one or more lipids include (R)-N,N,N-trimethyl-2,3-dioleyloxy-1-propanaminium chloride (DOTMA) and 1,2-dioleoyl-sn-glycero-3-phosphoethanolamine (DOPE). In certain embodiments, at physiological pH the overall charge ratio of positive charges to negative charges of the liposome is 1.3:2 (0.65).

In certain embodiments, the RNA vaccine includes an RNA molecule including, in the 5′43′ direction: (1) a 5′ cap; (2) a 5′ untranslated region (UTR); (3) a polynucleotide sequence encoding a secretory signal peptide; (4) a polynucleotide sequence encoding the one or more neoepitopes resulting from cancer-specific somatic mutations present in the tumor specimen; (5) a polynucleotide sequence encoding at least a portion of a transmembrane and cytoplasmic domain of a major histocompatibility complex (MHC) molecule; (6) a 3′ UTR including: (a) a 3′ untranslated region of an Amino-Terminal Enhancer of Split (AES) mRNA or a fragment thereof; and (b) non-coding RNA of a mitochondrially encoded 12S RNA or a fragment thereof; and (7) a poly(A) sequence.

In certain embodiments, the RNA molecule further includes a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction. In certain embodiments, the amino acid linker includes the sequence GGSGGGGSGG (SEQ ID NO: 21). In certain embodiments, the polynucleotide sequence encoding the amino acid linker includes the sequence

(SEQ ID NO: 19)
GGCGGCUCUGGAGGAGGCGGCUCCGGAGGC.

In certain embodiments, the RNA molecule further includes, in the 5′→3′ direction: at least a second linker-epitope module, wherein the at least second linker-epitope module includes a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-epitope module is different from the neoepitope of the second linker-epitope module. In certain embodiments, the RNA molecule includes 5 linker-epitope modules, wherein the 5 linker-epitope modules each encode a different neoepitope. In certain embodiments, the RNA molecule includes 10 linker-epitope modules, wherein the 10 linker-epitope modules each encode a different neoepitope. In certain embodiments, the RNA molecule includes 20 linker-epitope modules, wherein the 20 linker-epitope modules each encode a different neoepitope.

In certain embodiments, the RNA molecule further includes a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

In certain embodiments, the 5′ cap includes a D1 diastereoisomer of the structure:

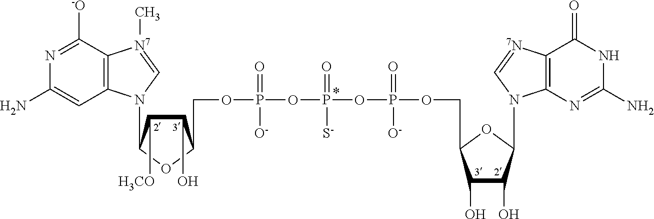

In certain embodiments, the 5′ UTR includes the sequence UUCUUCUGGUCCCCACAGACUCAGAGAGAACCCGCCACC (SEQ ID NO:5). In certain embodiments, the 5′ UTR includes the sequence

(SEQ ID NO: 3)
GGCGAACUAGUAUUCUUCUGGUCCCCACAGACUCAGAGAGAACCCGCC
ACC.

In certain embodiments, the secretory signal peptide includes the amino acid sequence MRVMAPRTLILLLSGALALTETWAGS (SEQ ID NO:9). In certain embodiments, the polynucleotide sequence encoding the secretory signal peptide includes the sequence

(SEQ ID NO: 7)
AUGAGAGUGAUGGCCCCCAGAACCCUGAUCCUGCUGCUGUCUGGCGCC
CUGGCCCUGACAGAGACAUGGGCCGGAAGC.

In certain embodiments, the at least portion of the transmembrane and cytoplasmic domain of the MHC molecule includes the amino acid sequence

(SEQ ID NO: 12)
IVGIVAGLAVLAVVVIGAVVATVMCRRKSSGGKGGSYSQAASSDSAQG
SDVSLTA.

In certain embodiments, the polynucleotide sequence encoding the at least portion of the transmembrane and cytoplasmic domain of the MHC molecule includes the sequence

(SEQ ID NO: 10)
AUCGUGGGAAUUGUGGCAGGACUGGCAGUGCUGGCCGUGGUGGUGAUC
GGAGCCGUGGUGGCUACCGUGAUGUGCAGACGGAAGUCCAGCGGAGGC
AAGGGCGGCAGCUACAGCCAGGCCGCCAGCUCUGAUAGCGCCCAGGGC
AGCGACGUGUCACUGACAGCC.

In certain embodiments, the 3′ untranslated region of the AES mRNA includes the sequence

CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACU CACCACCUCUGCUAGUUCCAGACACCUCC (SEQ ID NO:15). In certain embodiments, the non-coding RNA of the mitochondrially encoded 12S RNA includes the sequence CAAGCACGCAGCAAUGCAGCUCAAAACGCUUAGCCUAGCCACACCCCCACGGG AAACAGCAGUGAUUAACCUUUAGCAAUAAACGAAAGUUUAACUAAGCUAUAC UAACCCCAGGGUUGGUCAAUUUCGUGCCAGCCACACCG (SEQ ID NO:17). In certain embodiments, the 3′ UTR includes the sequence

(SEQ ID NO: 13)
CUCGAGCUGGUACUGCAUGCACGCAAUGCUAGCUGCCCCUUUCCCGUC
CUGGGUACCCCGAGUCUCCCCCGACCUCGGGUCCCAGGUAUGCUCCCA
CCUCCACCUGCCCCACUCACCACCUCUGCUAGUUCCAGACACCUCCCA
AGCACGCAGCAAUGCAGCUCAAAACGCUUAGCCUAGCCACACCCCCAC
GGGAAACAGCAGUGAUUAACCUUUAGCAAUAAACGAAAGUUUAACUAA
GCUAUACUAACCCCAGGGUUGGUCAAUUUCGUGCCAGCCACACCGAGA
CCUGGUCCAGAGUCGCUAGCCGCGUCGCU.

In certain embodiments, the poly(A) sequence includes 120 adenine nucleotides.

In certain embodiments, the RNA vaccine includes an RNA molecule including, in the 5′→3′ direction: the polynucleotide sequence GGCGAACUAGUAUUCUUCUGGUCCCCACAGACUCAGAGAGAACCCGCCACCAU GAGAGUGAUGGCCCCCAGAACCCUGAUCCUGCUGCUGUCUGGCGCCCUGGCCC UGACAGAGACAUGGGCCGGAAGC (SEQ ID NO:1); a polynucleotide sequence encoding the one or more neoepitopes resulting from cancer-specific somatic mutations present in the tumor specimen; and the polynucleotide sequence

(SEQ ID NO: 2)
AUCGUGGGAAUUGUGGCAGGACUGGCAGUGCUGGCCGUGGUGGUGAUC
GGAGCCGUGGUGGCUACCGUGAUGUGCAGACGGAAGUCCAGCGGAGGC
AAGGGCGGCAGCUACAGCCAGGCCGCCAGCUCUGAUAGCGCCCAGGGC
AGCGACGUGUCACUGACAGCCUAGUAACUCGAGCUGGUACUGCAUGCA
CGCAAUGCUAGCUGCCCCUUUCCCGUCCUGGGUACCCCGAGUCUCCCC
CGACCUCGGGUCCCAGGUAUGCUCCCACCUCCACCUGCCCCACUCACC
ACCUCUGCUAGUUCCAGACACCUCCCAAGCACGCAGCAAUGCAGCUCA
AAACGCUUAGCCUAGCCACACCCCCACGGGAAACAGCAGUGAUUAACC
UUUAGCAAUAAACGAAAGUUUAACUAAGCUAUACUAACCCCAGGGUUG
GUCAAUUUCGUGCCAGCCACACCGAGACCUGGUCCAGAGUCGCUAGCC
GCGUCGCU.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims

Before developing the methods and systems disclosed herein, various experiments were performed to understand, develop, and optimize the methods and systems. Specifically, experiments were performed in three phases: (1) to understand peristaltic pump flow rate pulsations (Example 1); (2) to develop and optimize dampener and peristaltic pump configurations to maintain a consistent steady flow rate as measured by level of pulsation (“LoP”) (Example 2); and (3) to optimize the dampener and peristaltic pump configurations to a flow process having fluids from two sources (Example 3). These experiments provided the opportunity to assess and quantify the effectiveness of a large number of parameters that impact the LoP.

The goal of these experiments was to a develop methods and systems that has an acceptable LoP, including, e.g., similarity to and/or improvement over other alternatives such as syringe pumps, and are simple to implement in an aseptic, closed, good manufacturing practices (“GMP”) environment using single use materials (i.e., material of construction and sterilization considerations for use with pharmaceutical formulations described herein). The materials of construction can be important as product contacting surfaces should not leach into the product (nor the product should extract any species from the materials). As such, such materials should be adequate for use in manufacturing or transferring pharmaceutical compositions, including the pharmaceutical compositions and formulations described herein, such as, for example, platinum cured silicone tubing.

### MEASUREMENTS AND CALCULATIONS USED IN THE EXAMPLES

Non product contacting flow meters (e.g., Keyence input model FD-XA1) were used to monitor the flow rate and LoP in the experiments described in the Examples. Sensor head FD-Xs8 was paired with clamp sets FD-XCR2 or FD-XCR1 based on target tubing outside diameters. Each Keyence meter was connected to a Keysight high speed data logger (e.g., U2541A) and the accompanying Keysight software was used to record data every 10 ms. Flow meters were used to measure the dynamic flow rate in the system and the LoP was determined using the dynamic flow rate in the system during steady state operation.

Flow meters were placed at a minimum of 10 inner diameters away from any changes in the flow path such as a dampener, connector, sharp turns, etc., to eliminate entrance effects from the flow measurement. Prior to each experiment, each meter was initialized, which sets a 0 mL/min flow rate. A minimum of seven seconds were recorded with a sample rate of 100 Hz. A representative second, typically the fourth or fifth second, was used for both pulsation calculations and data plots since multiple periods (i.e., the distance between flow rate peaks) were present in a second. In some embodiments, data was taken from a 10 second period once steady state flow was established.

Level of Pulsation: Level of pulsation (“LoP”) calculations were performed on the representative second that was used for data plots. This calculation is as follows:

(Maximum flow rate−Minimum flow rate)/Average flow rate×100.

Maximum and minimum flow rate percent: Maximum and minimum flow rate percentages were calculated as follows:

Maximum Flow Rate Percent=((Maximum flow rate−Average flow rate)/Average flow rate×100

Minimum Flow Rate Percent=((Average flow rate−Minimum flow rate)/Average flow rate×100

For all experiments, a single peristaltic pump was used. For experiments with two pump heads, a single pump was mounted with two pump heads. In experiments where there are four product lines in contact with rollers (dual head pump with two separate streams), a single pump with two heads was used and two lines were fed into a single pump head as opposed to using two separate pumps with two pump heads. Unless stated otherwise, the average flow rate of the pumps described in the Examples was around 50 mL/min.

### Example 1: Understanding Peristaltic Pump Flow Rate Pulsations

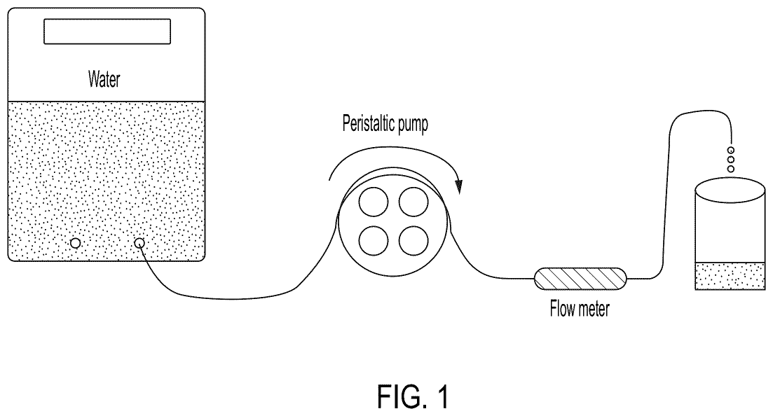

As described herein, the flowrate from a peristaltic pump can pulse or oscillate over time due to the nature of peristaltic pumps. See, e.g., Experiment 1 (as described in Table 1A (set up details of the Experiment) and Table 1B (results of Experiment 1) wherein the flowrate of water moving through a peristaltic pump was measured over a given period of time. Briefly, Experiment 1 was performed using a single pump head without a dampener using a 30 cm length of tubing downstream from the dampener. This setup represented an experimental baseline for the LoP expected for a peristaltic pump system without any attempt to reduce the flow rate variations. FIG. 1 illustrates the experimental setup for Experiment 1. The setup included a container of water, a peristaltic pump, and a flowmeter to measure the flow of water out of the peristaltic pump. The average flow rate of the pump was around 50 mL/min.

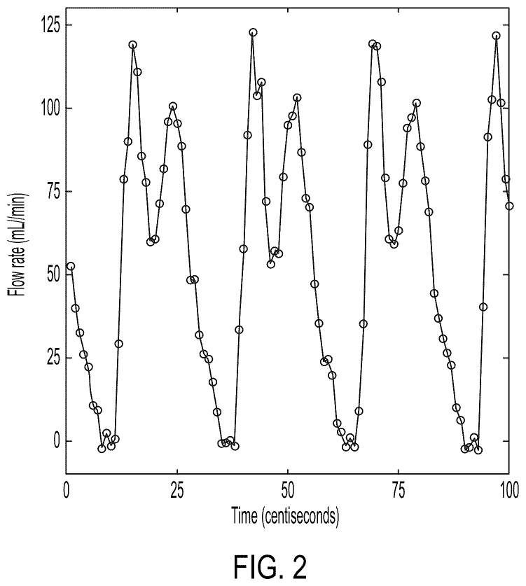

FIG. 2 illustrates the flowrate of the water through the peristaltic pump measured over time. As shown in FIG. 2, the flow rate significantly oscillates over time. Tables 1A and 1B provide additional details and a summary of results of Experiment 1.

TABLE 1A

| | | No. of | No. | | Tubing | Tubing | Tubing | |
|---|---|---|---|---|---|---|---|---|
| | | rollers | pump | | outlet | inlet ID, | outlet ID, | |
| Ex. | Pump | per pump | heads | | length | OD, W in mm | OD, W in mm | Tee used |
| No. | (part #) | head | used | Dampener | (cm) | (part #) | (part #) | (part #) |
| 1 | Masterflex | 4 | 1 | None | 30 | 3.2, 6.4, 1.6 | 3.2, 6.4, 1.6 | N/A |
| | pump with | | | | | (STHT-C- | (STHT-C- | |
| | easy load | | | | | 125-2) | 125-2) | |
| | head (HV- | | | | | | | |
| | 77921-75) | | | | | | | |

TABLE 1B

| | Average | Maximum | Minimum | Maximum | Minimum | | Relative | |
|---|---|---|---|---|---|---|---|---|
| | flow | flow | flow | flow | flow | | standard | |
| Ex. | rate | rate | rate | rate | rate | Standard | deviation | Level of |
| No. | (mL/min) | (mL/min) | (mL/min) | (%) | (%) | deviation | (%) | pulsation |
| 1 | 53.45 | 122.66 | −2.88 | 129.49 | 105.39 | 39 | 72 | 235 |

The minimum flow rate observed in Experiment 1 reached values below 0; however, this was attributed to a combination of: (1) inherent solution suck-back; and (2) small degree of sensor measurement error.

### Example 2: Optimizing Dampener Configurations in Peristaltic Pump Systems to Induce a Consistent Steady Flow Rate

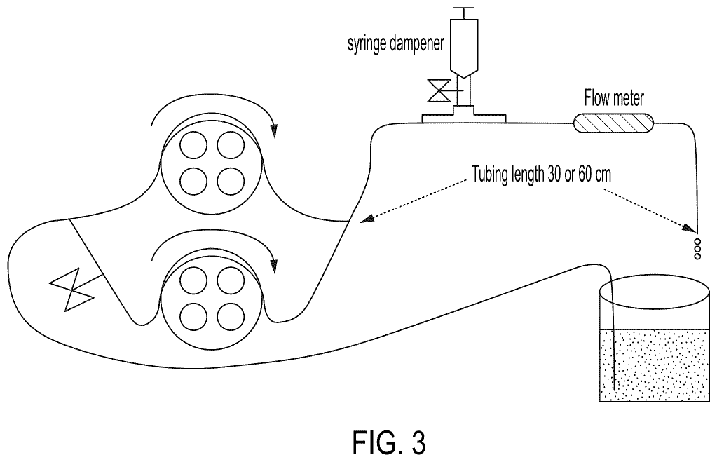

One potential way of reduce the pulsations from a peristaltic pump is to use a dampener. As such, various designs and configurations of dampeners after the peristaltic pump were examined. Air and/or gas can be used as the dampener because it is highly compressible. As such, the designs of the dampeners can be either open directly to air or they can have a membrane in contact with the fluid and the air. FIG. 3 illustrates the experimental setup for measuring flowrates using one or two peristaltic pump heads and a dampener after the peristaltic pump(s). A dampener can reduce or eliminate the variations in pressure and flow produced by peristaltic pumps. Specifically, a dampener can absorb the extra fluid during the peak flow rate and release it on the downside to smooth the flowrate. For Experiments 1-3, one pump head was used. All other Experiments described herein used two pump heads.

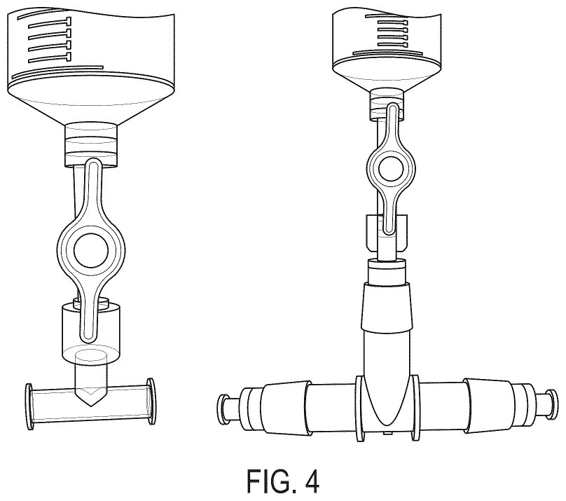

The experimental setup in FIG. 3 includes a dampener after the peristaltic pumps. As shown in FIG. 3, the dampener is a syringe dampener with a tee connector. The tee connector can be a fitting which is T-shaped having two outlets at 90 degrees to the connection to the main line. It can be a short piece of pipe with a lateral outlet. The relative size/opening of the tee can impact dampening efficiency. The dampener and tee connector are fluidly connected to the outlet tubing from the peristaltic pumps as shown in FIG. 3. One example of a dampener is a syringe dampener. Tubing after a peristaltic pump can be fluidly connected to a syringe dampener. In some embodiments, the tubing after a peristaltic pump can be fluidly connected to a tee connector which is fluidly connected to a syringe. Examples of images of a syringe dampener with various tee connectors are shown in FIG. 4. The plunger of the syringe can be pulled back such that the volume of air inside the syringe can be adjusted for a given use. Air inside the syringe can act as a space buffer where extra fluid can be temporarily stored when the peristaltic pump is active. This temporary storage can increase the air pressure, which pushes the fluid out when the flowrate from the peristaltic pump decreases.

Several experiments (i.e. Experiments 2-16) using water in accordance with the experimental setup shown in FIG. 3 were conducted by adjusting the following variables: (1) number of pump heads used (using N number of pump heads can multiply the flow rate by N, roughly, given the same settings on the pump. Most pumps can be tuned to many flow rates depending on the minimum and maximum RPM of the rollers for that pump); (2) syringe dampener or no syringe dampener; (3) the size of the tee connector used (the majority of the time, the tee may not affect the flow rate unless in a rare scenario where the tee has a small opening and is now limiting the flow rate); (4) the tubing outlet length (outlet tubing length can affect pulsations. The longer the outlet, the lower LoP can be because the flexibility of the tubing naturally dampens the system); and (5) the volume of air in the syringe. Additional details and results of these experiments (Experiments 2-9 and 12-16) are summarized in Tables 2A (set up details of the experiments) and Table 2B (results of the experiments).

TABLE 2A

| | | No. of | No. | | Tubing | Tubing | Tubing | |
|---|---|---|---|---|---|---|---|---|
| | | rollers | pump | | outlet | inlet ID, | outlet ID, | |
| Ex. | Pump | per pump | heads | | length | OD, W in mm | OD, W in mm | Tee used |
| No. | (part #) | head | used | Dampener | (cm) | (part #) | (part #) | (part #) |
| 2 | Masterflex | 4 | 1 | 60 mL | 30 | 3.2, 6.4, 1.6 | 3.2, 6.4, | Cole |
| | pump with | | | Syringe | | (STHT-C- | 1.6 | Parmer |
| | easy load | | | (volume of air | | 125-2) | (STHT-C- | Female- |
| | head (HV- | | | of 60 mL) | | | 125-2) | female- |
| | 77921-75) | | | | | | | female |
| | | | | | | | | Luer |
| | | | | | | | | (EW- |
| | | | | | | | | 45511-00) |
| 3 | Masterflex | 4 | 1 | 60 mL | 60 | 3.2, 6.4, 1.6 | 3.2, 6.4, | Cole |
| | pump with | | | Syringe | | (STHT-C- | 1.6 | Parmer |
| | easy load | | | (volume of | | 125-2) | (STHT-C- | Female- |
| | head (HV- | | | air of 60 mL) | | | 125-2) | female- |
| | 77921-75) | | | | | | | female |
| | | | | | | | | Luer |
| | | | | | | | | (EW- |
| | | | | | | | | 45511-00) |
| 4 | Masterflex | 4 | 2 | None | 30 | 3.2, 6.4, 1.6 | 3.2, 6.4, | N/A |
| | pump with | | | | | (STHT-C- | 1.6 | |
| | easy load | | | | | 125-2) | (STHT-C- | |
| | head (HV- | | | | | | 125-2) | |
| | 77921-75) | | | | | | | |
| 5 | Masterflex | 4 | 2 | 60 mL | 30 | 3.2, 6.4, 1.6 | 3.2, 6.4, | Cole |
| | pump with | | | Syringe | | (STHT-C- | 1.6 | Parmer |
| | easy load | | | (volume of | | 125-2) | (STHT-C- | Female- |
| | head (HV- | | | air of 60 mL) | | | 125-2) | female- |
| | 77921-75) | | | | | | | female |
| | | | | | | | | Luer |
| | | | | | | | | (EW- |
| | | | | | | | | 45511-00) |
| 6 | Masterflex | 4 | 2 | 60 mL | 60 | 3.2, 6.4, 1.6 | 3.2, 6.4, | Cole |
| | pump with | | | Syringe | | (STHT-C- | 1.6 | Parmer |
| | easy load | | | (volume of air | | 125-2) | (STHT-C- | Female- |
| | head (HV- | | | of 60 mL) | | | 125-2) | female- |
| | 77921-75) | | | | | | | female |
| | | | | | | | | Luer |
| | | | | | | | | (EW- |
| | | | | | | | | 45511-00) |
| 7 | Masterflex | 4 | 2 | 60 mL | 60 | 3.2, 6.4, 1.6 | 3.2, 6.4, | Cole |
| | pump with | | | Syringe | | (STHT-C- | 1.6 | Parmer |
| | easy load | | | (volume of air | | 125-2) | (STHT-C- | Female- |
| | head (HV- | | | of 60 mL) | | | 125-2) | female- |
| | 77921-75) | | | | | | | female |
| | | | | | | | | Luer |
| | | | | | | | | (EW- |
| | | | | | | | | 45511-00) |
| 8 | Masterflex | 4 | 2 | 60 mL | 60 | 3.2, 6.4, 1.6 | 3.2, 6.4, | Cole |
| | pump with | | | Syringe | | (STHT-C- | 1.6 | Parmer |
| | easy load | | | (volume of air | | 125-2) | (STHT-C- | Female- |
| | head (HV- | | | of 60 mL) | | | 125-2) | female- |
| | 77921-75) | | | | | | | female |
| | | | | | | | | Luer |
| | | | | | | | | (EW- |
| | | | | | | | | 45511-00) |
| 9 | Masterflex | 4 | 2 | 60 mL | 60 | 3.2, 6.4, 1.6 | 3.2, 6.4, | US Plastic |
| | pump with | | | Syringe | | (STHT-C- | 1.6 | ⅜-⅜- |
| | easy load | | | (volume of air | | 125-2) | (STHT-C- | ⅜″ tee |
| | head (HV- | | | of 60 mL) | | | 125-2) | (62210) |
| | 77921-75) | | | | | | | |
| 12 | Masterflex | 4 | 2 | 60 mL | 60 | 3.2, 6.4, 1.6 | 3.2, 6.4, | US Plastic |
| | pump with | | | Syringe | | (STHT-C- | 1.6 | ⅜-⅜- |
| | easy load | | | (volume of air | | 125-2) | STHT-C- | ⅜″ tee |
| | head (HV- | | | of 60 mL) | | | 125-2) | (62210) |
| | 77921-75) | | | | | | | |
| 13 | Masterflex | 4 | 2 | 60 mL | 60 | 3.2, 6.4, 1.6 | 3.2, 6.4, | US Plastic |
| | pump with | | | Syringe | | (STHT-C- | 1.6 | ⅜-⅜- |
| | easy load | | | (syringe at 40 | | 125-2) | (STHT-C- | ⅜″ tee |
| | head (HV- | | | mL position) | | | 125-2) | (62210) |
| | 77921-75) | | | | | | | |
| 14 | Masterflex | 4 | 2 | 60 mL | 60 | 3.2, 6.4, 1.6 | 3.2, 6.4, | US Plastic |
| | pump with | | | Syringe | | (STHT-C- | 1.6 | ⅜-⅜- |
| | easy load | | | (syringe at 20 | | 125-2) | (STHT-C- | ⅜″ tee |
| | head (HV- | | | mL position) | | | 125-2) | (62210) |
| | 77921-75) | | | | | | | |
| 15 | Masterflex | 4 | 2 | 60 mL | 60 | 3.2, 6.4, 1.6 | 3.2, 6.4, | US Plastic |
| | pump with | | | Syringe | | (STHT-C- | 1.6 | ⅜-⅜- |
| | easy load | | | (syringe at 10 | | 125-2) | (STHT-C- | ⅜″ tee |
| | head (HV- | | | mL position) | | | 125-2) | (62210) |
| | 77921-75) | | | | | | | |
| 16 | Masterflex | 4 | 2 | 60 mL | 60 | 3.2, 6.4, 1.6 | 3.2, 6.4, | US Plastic |
| | pump with | | | Syringe | | (STHT-C- | 1.6 | ⅜-⅜- |
| | easy load | | | (syringe at 5 | | 125-2) | (STHT-C- | ⅜″ tee |
| | head (HV- | | | mL position) | | | 125-2) | (62210) |
| | 77921-75) | | | | | | | |

TABLE 2B

| | Average | Maximum | Minimum | Maximum | Minimum | | Relative | |
|---|---|---|---|---|---|---|---|---|
| | flow | flow | flow | flow | flow | | standard | |
| Ex. | rate | rate | rate | rate | rate | Standard | deviation | Level of |
| No | (mL/min) | (mL/min) | (mL/min) | (%) | (%) | deviation | (%) | pulsation |
| 2 | 52.16 | 62.28 | 44.22 | 19.40 | 15.22 | 5 | 10 | 35 |
| 3 | 52.19 | 58.19 | 44.97 | 11.50 | 13.84 | 3 | 6 | 25 |
| 4 | 52.35 | 76.31 | 5.92 | 45.77 | 88.69 | 20 | 38 | 134 |
| 5 | 52.8 | 58.54 | 46.41 | 10.88 | 12.10 | 3 | 6 | 23 |
| 6 | 52.75 | 57.96 | 46.98 | 9.88 | 10.94 | 3 | 5 | 21 |
| 7 | 52.02 | 57.85 | 43.36 | 11.19 | 16.67 | 3 | 6 | 28 |
| 8 | 52.11 | 56.01 | 45.89 | 7.48 | 11.94 | 2 | 5 | 19 |
| 9 | 52.02 | 55.32 | 46.06 | 6.35 | 11.45 | 2 | 4 | 18 |
| 12 | 50.85 | 54.34 | 43.24 | 6.87 | 14.96 | 2 | 4 | 22 |
| 13 | 51.31 | 55.72 | 45.20 | 8.59 | 11.92 | 2 | 4 | 21 |
| 14 | 50.8 | 56.41 | 45.2 | 11.04 | 11.03 | 3 | 5 | 22 |
| 15 | 51.37 | 57.96 | 42.32 | 12.83 | 17.62 | 3 | 6 | 30 |
| 16 | 50.19 | 59.86 | 36.46 | 19.26 | 27.37 | 6 | 12 | 47 |

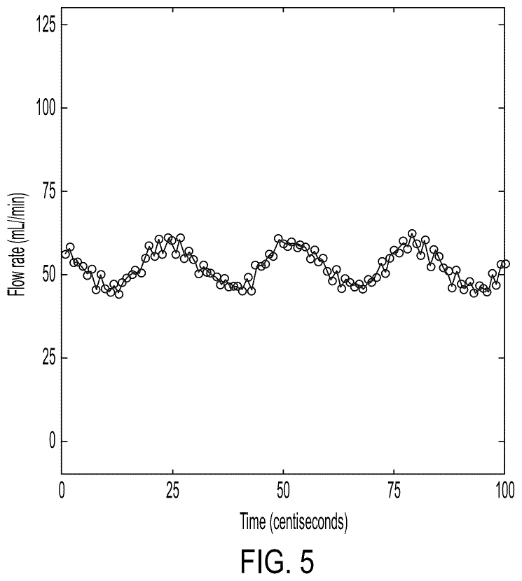

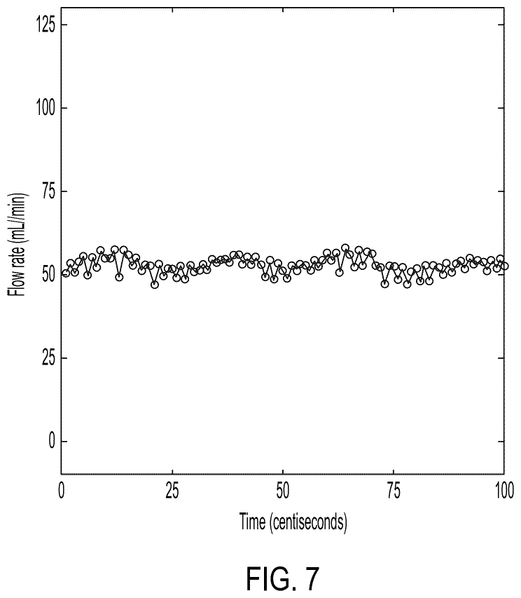

The flow rate over time achieved in Experiments 2, 4, and 6 are shown in FIGS. 5, 6, and 7, respectively. As shown in these figures, the flowrate of Experiment 6 (FIG. 7) using two pump heads, a 60 mL syringe dampener at the largest air volume (i.e., 60 mL), and a tubing outlet length of 60 cm significantly reduced pulsations throughout the experiment.

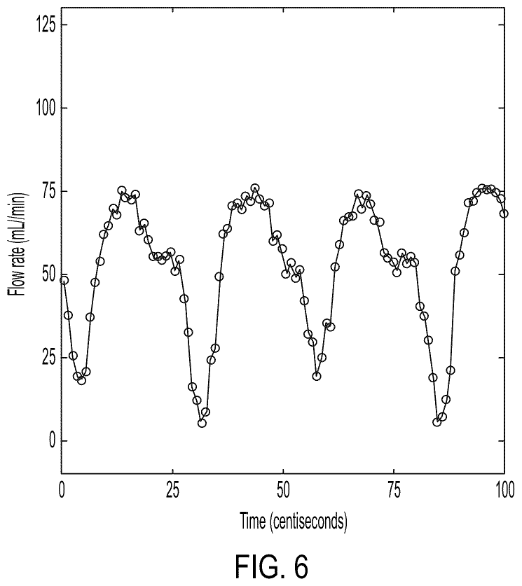

Experiment 4 assessed the impact of a second pump head (e.g., a single pump with two pump heads attached driven by the same motor) This was conducted by opening a valve adjacent to the second pump head. See, FIG. 3. Although the LoP decreased to 134, there were still flow rate variations as shown in FIG. 6. Adding subsequent pump heads would likely further decrease LoP; however, this would increase complication of the product flow path and tubing kit for the system and many peristaltic pumps only have two fixed heads.

Experiments 2 and 5 added a syringe dampener. This was accomplished by opening the valve attached to the tee and 60 mL syringe. See, FIG. 3. Prior to opening the valve, the syringe was adjusted to the largest position (60 mL mark), connected to a luer lock tee, and the tee was connected inline to the outlet tubing in which the solution would flow without any sharp turns. The LoP values greatly decreased to values of 35 and 23 for Experiments 2 and 5, respectively. As such, the syringe dampener proved effective, but slight pulsations were still observed.

Another potential method to reduce flow rate variations is to increase the outlet tubing length. In Experiments 3 and 6, the outlet tubing length was changed from 30 cm to 60 cm when compared to that of Experiments 2 and 5. The LoP values decreased from 35 and 23 (see Table 2B Experiments 2 and 5, respectively) to 25 and 21 (see Table 2B, Experiments 3 and 6, respectively).

Experiments 12-16 explored the volume of air within the 60 mL syringe dampener to better understand the minimum volume that can effectively dampen the flow. For these experiments, the position of the plunger was adjusted to the volumetric tick marks of 60, 40, 10, and 5 cc, and the LoPs were 22, 21, 22, 30, and 47, respectively, as reported in Table 2B. As such, there was no significant difference between LoPs until the plunger position was adjusted to 10 cc and 5 cc in which the dampener became less effective.

During the experiments, the effectiveness of the dampener was impacted by the location of the air-liquid interface in the dampener/tee system. Accordingly, Experiment 7 was conducted using the same setup as Experiment 6, but unlike Experiment 6 in which the solution remained in the tee during testing, it was observed during Experiment 7 that solution crept up past the tee into the valve where it remained for the duration of the experiment. The LoP increased from 21 to 28 between Experiments 6 and 7 (see Table 2B) suggesting that the air-liquid interface surface area could impact the effectiveness of the dampener. The inner diameter of the luer tee is 3.1 mm while the inner diameter of the luer valve is 4.1 mm. The luer tee was determined to be not robust so a larger tee with an inner diameter of 9.5 mm was tested in Experiment 9. The LoP decreased to 18 confirming that the air-liquid interface surface area in the dampener/tee system is a parameter to control.

The ability to implement a peristaltic pump dampener can depend on many variables including the material of construction, ability to conduct sterilization, and geometry (i.e., how quickly the air pressure will pressurize in respond to fluid pulses). Experiments 1-16 d escribed above demonstrated that flow rate pulsations can be reduced using a dampener, but a syringe dampener may not meet the criteria (e.g., LoP and/or sterility robustness for GMP use as explained below) for use with the pharmaceutical compositions described herein, including, e.g., pharmaceutical compositions comprising RNA or lipids, such as an RNA vaccine. Experiments 1-16 provided proof of concept that LoP can be reduced and additional parameters were further optimized to increase the observed reductions in LoP. One such parameter was to develop a dampener that could be easily implemented for use with the pharmaceutical formulations comprising RNA or lipids, such as an RNA vaccine.

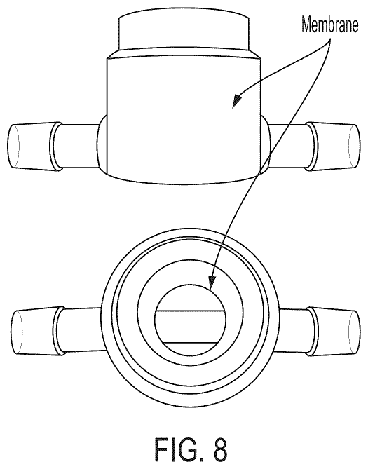

Besides a syringe dampener, another type of pulsation dampener that can be used is a membrane dampener. As such, the tubing after a peristaltic pump can be fluidly connected to a membrane dampener. In some embodiments, the tubing after a peristaltic pump can be fluidly connected to a tee connector which is fluidly connected to a membrane. An exemplary membrane dampener is shown in FIG. 8. Membrane dampeners are similar to other dampeners disclosed herein in that the compressible gas is performing the dampener with the membrane as a barrier. In some cases, an enclosed system with a compressible gas dampens with the membrane acting as a barrier between the gas and solution can be used. For the experiments described herein, the gas was the atmosphere so while it was not enough in this scenario; an enclosed system can be designed with a compressible gas and a flexible membrane. Fine tuning of the membrane (flexibility, surface area, durometers, etc.) and of the compressible gas can help reduce the LoP.

To examine the potential impact of a membrane dampener, an experiment using water in accordance with the experimental setup shown in FIG. 3 was conducted but replaced the syringe dampener with a membrane dampener. Additional details and results of this experiment (Experiment 10) are summarized in Tables 3A (set up details of the experiment) and Table 3B (results of the experiment).

TABLE 3A

| | | No. of | No. | | Tubing | Tubing | Tubing | |
|---|---|---|---|---|---|---|---|---|
| | | rollers | pump | | outlet | inlet ID, | outlet ID, | |
| Ex. | Pump | per pump | heads | | length | OD, W in mm | OD, W in mm | Tee used |
| No. | (part #) | head | used | Dampener | (cm) | (part #) | (part #) | (part #) |
| 10 | Masterflex | 4 | 2 | Flexible | 60 | 3.2, 6.4, 1.6 | 3.2, 6.4, 1.6 | N/A |
| | pump with | | | membrane | | (STHT-C- | (STHT-C- | |
| | easy load | | | (Part was | | 125-2) | 125-2) | |
| | head (HV- | | | taken from | | | | |
| | 77921-75) | | | an existing | | | | |
| | | | | kit, part # | | | | |
| | | | | unknown) | | | | |

TABLE 3B

| | | Maximum | Minimum | Maximum | Minimum | | Relative | |
|---|---|---|---|---|---|---|---|---|
| | Average | flow | flow | flow | flow | | standard | |
| Ex | flow rate | rate | rate | rate | rate | Standard | deviation | Level of |
| No. | (mL/min) | (mL/min) | (mL/min) | (%) | (%) | deviation | (%) | pulsation |
| 10 | 50.66 | 65.21 | 32.32 | 28.72 | 36.21 | 9 | 17 | 65 |

Experiment 10 assessed a dampener of a tee with a flexible membrane. In theory, the membrane in combination with atmospheric pressure should act as a dampener. However, the LoP of this system was 65 suggesting that the membrane was only slightly effective at reducing flow rate pulsations, and still too high for use with the pharmaceutical compositions described herein, including, e.g., pharmaceutical compositions comprising RNA or lipids, such as an RNA vaccine.

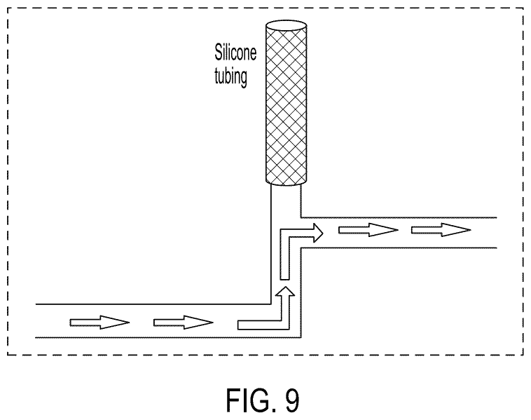

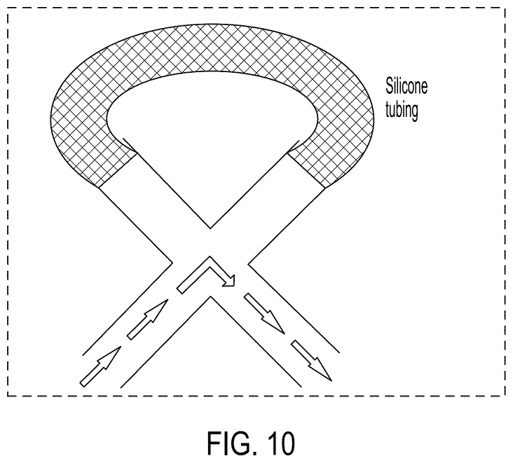

Besides a membrane or a syringe, gas-filled tubing itself can potentially be used as a pulsation dampener. As such, the tubing after a peristaltic pump can be fluidly connected to a tubing dampener. Exemplary configurations of tubing dampeners are shown in FIGS. 9 and 10. As illustrated in FIG. 9, a tubing dampener can be a tee connector tubing dampener. When such a tee connector tubing dampener is used, the tubing after a peristaltic pump can be fluidly connected to a tee connector which is fluidly connected to the tubing dampener. The tubing of the tubing dampener can be made out of the same or different material as the tubing after the peristaltic pump. In some embodiments, the tubing of the tubing dampener can be silicone. In some embodiments, the tubing dampener can include a clamp or other object such that the end of the tubing dampener opposite the end that is fluidly connected to the tee connector is closed. The tubing dampener works similarly to other dampeners in that the enclosed gas can perform the dampening.

FIG. 10 illustrates an example of a tubing dampener with a cross or 4-way connector. In place of a tee connector, the 4-way connector (dampening effectiveness can depend on the valve opening dimensions) in FIG. 10 allows for both ends of the tubing dampener to be connected to the 4-way connector instead of using a clamp or other device to close one end of the tubing dampener. When a 4-way tubing dampener is used, the tubing after a peristaltic pump can be fluidly connected to a 4-way connector which is fluidly connected to the tubing dampener. As such, fluid from the peristaltic pump can enter one of the openings of the 4-way connector and exit another opening, whereas the tubing dampener can be connected to the other two unused openings such that both ends of the tubing dampener are fluidly connected to the 4-way connector. The 4-way tubing dampener can work similarly to the other dampeners in that the gas enclosed can perform the dampening.

To examine the potential impact of a tee connector tubing dampener (as shown in FIG. 9) or a 4-way tubing dampener (as shown in FIG. 10) experiments using water in accordance with the experimental setup shown in FIG. 3 were conducted but replaced the syringe dampener with a tee connector tubing dampener or a 4-way connector tubing dampener. Additional details and results of these experiments (Experiments 11, 17 and 18) are summarized in Tables 4A (set up details of the experiments) and Table 4B (results of the experiments).

TABLE 4A

| | | No. of | No. | | Tubing | Tubing | Tubing | |
|---|---|---|---|---|---|---|---|---|
| | | rollers | pump | | outlet | inlet ID, | outlet ID, | |
| Ex. | Pump | per pump | heads | | length | OD, W in mm | OD, W in mm | Tee used |
| No. | (part #) | head | used | Dampener | (cm) | (part #) | (part #) | (part #) |
| 11 | Masterflex | 4 | 2 | 30 cm | 60 | 3.2, 6.4, 1.6 | 3.2, 6.4, 1.6 | N/A |
| | pump with | | | legnth thin- | | (STHT-C- | (STHT-C- | |
| | easy load | | | wall tubing | | 125-2) | 125-2) | |
| | head (HV- | | | 5/16″ OD, | | | | |
| | 77921-75) | | | ¼″ ID | | | | |
| | | | | (STHT-C- | | | | |
| | | | | 250-1) | | | | |
| 17 | Masterflex | 4 | 2 | 42 cm | 60 | 3.2, 6.4, 1.6 | 3.2, 6.4, 1.6 | US Plastic |
| | pump with | | | length of | | (STHT-C- | (STHT-C- | ⅜-⅜-⅜″ |
| | easy load | | | ⅜″ ID Si | | 125-2) | 125-2) | tee |
| | head (HV- | | | tubing and | | | | (62210) |
| | 77921-75) | | | tube clamp | | | | |
| | | | | (STHT-C- | | | | |
| | | | | 375-4 | | | | |
| | | | | [tubing]) | | | | |
| | | | | (EW- | | | | |
| | | | | 06833-00 | | | | |
| | | | | [clamp]) | | | | |
| 18 | Masterflex | 4 | 2 | 42 cm | 60 | 3.2, 6.4, 1.6 | 3.2, 6.4, 1.6 | US Plastic |
| | pump with | | | length of | | (STHT-C- | (STHT-C- | ⅜″ 4-way |
| | easy load | | | ⅜″ ID Si | | 125-2) | 125-2) | connector |
| | head (HV- | | | tubing in | | | | (64104) |
| | 77921-75) | | | loop | | | | |
| | | | | (STHT-C- | | | | |
| | | | | 375-4 | | | | |
| | | | | [tubing]) | | | | |

TABLE 4B

| | Average | Maximum | Minimum | Maximum | Minimum | | Relative | |
|---|---|---|---|---|---|---|---|---|
| | flow | flow | flow | flow | flow | | standard | |
| Ex. | rate | rate | rate | rate | rate | Standard | deviation | Level of |
| No. | (mL/min) | (mL/min) | (mL/min) | (%) | (%) | deviation | (%) | pulsation |
| 11 | 49.82 | 86.37 | 10.69 | 73.38 | 78.54 | 23 | 47 | 152 |
| 17 | 51.07 | 55.55 | 46.06 | 8.77 | 9.81 | 2 | 5 | 19 |
| 18 | 50.15 | 55.61 | 45.95 | 10.89 | 8.38 | 2 | 4 | 19 |

As a preliminary matter, Experiment 11 replaced the tee/dampener with 30 cm of thin-walled flexible tubing (7.9 mm OD, 0.8 mm wall thickness). The concept of using thin-walled flexible tubing as a dampener builds upon Experiments 3 and 6 (see Table 2B) in which a longer outlet length of tubing reduced the LoP. Not being bound by theory, the fluid would get dampened by the tubing and as flexibility of the tubing increases (i.e., going from rigid tubing to flexible tubing), so does the dampening effect. From this, a shorter length of thin-walled flexible tubing may achieve the same LoP compared to the tubing used in Experiments 3 and 6. To test this hypothesis, Experiment 11 was performed and with 30 cm of thin-walled tubing, the LoP increased from 134 (see Table 2B, Experiment 4, no dampener) to 152 (see Table 3B). Although an increase in LoP was initially observed by using thin-walled flexible tubing, the concept behind using tubing as a dampener was further explored as described further in Experiments 17 and 18. If an enclosed volume of air within a piece of tubing could replace that of a syringe and effectively reduce the LoP, many process requirements, such as sterilization and solution hold up, could be met.

Accordingly, Experiment 17 utilized a dead-ended (i.e, clamped) piece of silicone tubing (i.e. a tee connector tubing dampener as shown in FIG. 9), in place of the syringe, in which the tubing length was 42 cm (approximately 30 cc of air within the tubing). In addition, the position of the tee was changed to make it easier to mount, but this was hypothesized to have no effect on the dampener. As reported in Table 3B, the observed LoP was 19, which was comparable to the values with a syringe dampener (see, e.g., Table 2B). These results showed that the level of dampening of a dead-ended tubing is comparable to that of a syringe dampener. As previously described, the elegance of using a dead-ended tubing, instead of a syringe, is that such a system can meet the GMP process requirement for pharmaceutical compositions and formulations. In some embodiments, the dampener can be an open-ended (i.e., open to atmosphere) tubing dampener.

Experiment 18 replaced the tee with a four-way connector positioned like an “X” or “cross” (as shown in FIG. 10) in which two ports were connected with a single piece of silicone tubing forming a loop and the other two ports were used for fluid flow. The goal of Experiment 18 was to assess if there was any advantage between this dampener as compared to Experiment 17. The LoP of Experiment 18 was identical to that of Experiment 17. See Table 3B.

Although we observed significant reductions in LoP using in several dampener and tubing kit configurations—even achieving LoP values as lows as 18 to 22—the level of pulsation and oscillation is still not optimal for mixing and/or manufacturing the pharmaceutical compositions described herein, including, e.g., pharmaceutical compositions comprising RNA or lipids, including lipoplexes or liposomes, such as an RNA vaccine, and generally higher than the LoP values observed when using alternative syringe pump configurations (See Tables 5A and 5B, Examples 24-26). Accordingly, additional parameters were evaluated to reduce LoP values even further. Notwithstanding our goals to reduce LoP values, a skilled person would appreciate that the reduction of LoP observed in Example 2 may be suitable for other applications and pharmaceutical compositions, including, e.g., transferring and/or filing of pharmaceutical compositions into containers such as bags or vials.

### Example 3: Application of Dampener Configurations to Flow Processes Having Two Fluid Sources

Experiments 1-18 were mostly directed towards systems with one fluid source. However, peristaltic pumps can also be used in systems with more than one fluid source. One such example is when two pharmaceutical compositions are mixed or combined to form a final pharmaceutical composition, including, e.g., when a first pharmaceutical composition comprising an RNA or RNA vaccine is combined with a second pharmaceutical composition comprising one or more lipids to form a final pharmaceutical composition comprising RNA-lipoplexes or RNA liposomes. Because pharmaceutical compositions can include delicate and contain expensive ingredients, the amount of these ingredients used in the final pharmaceutical composition or formulation can be critical to whether the final pharmaceutical composition will be effective, safe, and cost-effective. Because the ingredients of a final pharmaceutical composition originate from different sources or containers, it can be important that the flowrates of these ingredients or intermediate pharmaceutical compositions in a peristaltic pump system are not pulsed such that the ingredients or intermediate pharmaceutical compositions can not be effectively mixed with the proper proportions for the final pharmaceutical composition comprising the mixture of the intermediate pharmaceutical compositions to be effective.

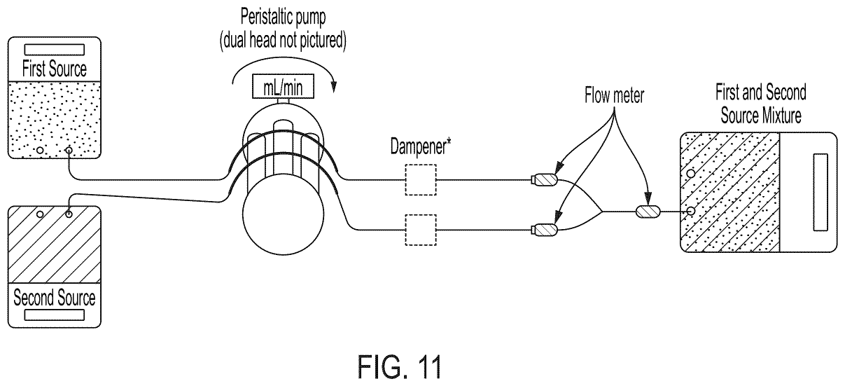

Because the pharmaceutical compositions described herein, including, e.g., pharmaceutical compositions comprising RNA or lipids, such as an RNA vaccine, are often mixed to create a final pharmaceutical composition comprising RNA-lipoplexes or RNA liposomes, experiments evaluating two different fluid sources with two peristaltic pumps were performed. In particular, experiments were conducted to evaluate the type of dampener that could achieve consistent flow rates across both the peristaltic pumps. FIG. 11 illustrates the experimental setup for measuring flowrates of a two fluid source system using one peristaltic pump and a dampener(s) after the peristaltic pump(s). Although there is only one peristaltic pump shown in FIG. 11, in some embodiments, the peristaltic pump can be a dual head peristaltic pump or a peristaltic pump with more than one head. As such, tubing from each fluid source can be attached to a head of the dual head peristaltic pump such that only one peristaltic pump is required. In some embodiments, each of the fluid sources can have their own peristaltic pump. However, for the experiments conducted in Experiments 19-20 the inlet from each source was split into two streams such that the each pump head of the dual pump head for each of the peristaltic pumps was used. Experiments 19 and 20 used a single peristaltic pump but the pump had two heads driven by the motor. In Experiments 21-22, only one of the pump heads of each dual head peristaltic pump was used.

Besides a different setup when compared to FIG. 3, additional changes were made to the experimental setup of FIG. 11. First, the peristaltic pump used was a Watson Marlow Flexicon PD121 and the tubing outlet dimensions were 2.4 mm ID instead of 3.2 mm ID. Experiment 20 established a baseline for the LoP observed in this setup without the use of a dampener for the two inlet lines. The minimum flow rate was around 80 mL/min for each inlet, which is different from the around 50 mL/min previously used. As shown in FIG. 11, flow meters were placed downstream of both inlet pumps as well as downstream of the Y-connector.

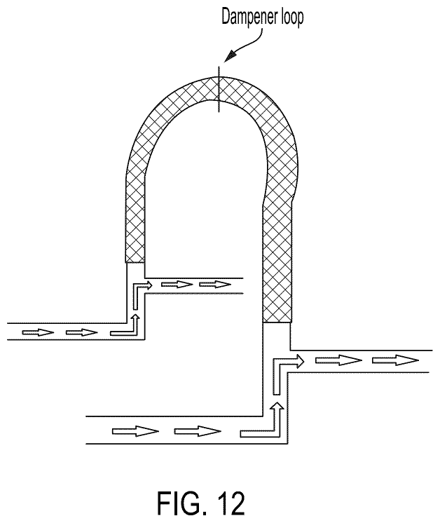

Experiment 19 implemented a dampener. In some embodiments, the dampeners could be two individual dead-ended tubing dampeners. However, Applicants discovered that a single tubing dampener can be used simultaneously on both inlets in the forming of dampening tubing loop. FIG. 12 illustrates an example of a dampener loop. The dampening loop can be connected to both tee connectors on the post-pump inlet lines. In addition, the dampening loop was mounted above the flow path to prevent solution from entering the loop.

Experiments 21 and 22 assessed the possibility of simplifying the tubing kit even further by utilizing a single pump head for each inlet as opposed to having a dual pump head. This can eliminate the need for a Y-connector directly upstream and downstream of the pump heads. However, the Y-connector involved in mixing the two separate inlets would still be required.

Additional details and results of Experiments 19-22 using the setup in FIG. 11 are summarized in Tables 5A (set up details of the experiments) and Table 5B (results of the experiments).

Experiments 24, 25, and 26 were conducted using two types of syringe pumps with the goal to directly compare LoP values to those observed in Experiment 19 (peristaltic pump with loop dampener). Additional details and results of Experiments 24-26 are summarized in Tables 5A (set up details of the experiments) and Table 5B (results of the experiments).

TABLE 5A

| | | No. of | No. | | Tubing | Tubing | Tubing | |
|---|---|---|---|---|---|---|---|---|
| | | rollers | pump | | outlet | inlet ID, | outlet ID, | |
| Ex. | Pump | per pump | heads | | length | OD, W in mm | OD, W in mm | Tee used |
| No. | (part #) | head | used | Dampener | (cm) | (part #) | (part #) | (part #) |
| 19.1 | Watson | 6 | 2 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 19.2 | Watson | 6 | 2 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 19.3 | Watson | 6 | 2 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 20.1 | Watson | 6 | 2 | None | 30 | 3.2, 6.4, 1.6 | 2.4, 5.6, 1.6 | N/A |
| | Marlow | | | | | (STHT-C- | (STHT-C- | |
| | Flexicon | | | | | 125-2) | 093-2) | |
| | (PD12I) | | | | | | | |
| 20.2 | Watson | 6 | 2 | None | 30 | 3.2, 6.4, 1.6 | 2.4, 5.6, 1.6 | N/A |
| | Marlow | | | | | (STHT-C- | (STHT-C- | |
| | Flexicon | | | | | 125-2) | 093-2) | |
| | (PD12I) | | | | | | | |
| 20.3 | Watson | 6 | 2 | None | 30 | 3.2, 6.4, 1.6 | 2.4, 5.6, 1.6 | N/A |
| | Marlow | | | | | (STHT-C- | (STHT-C- | |
| | Flexicon | | | | | 125-2) | 093-2) | |
| | (PD12I) | | | | | | | |
| 21.1 | Watson | 6 | 1 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 21.2 | Watson | 6 | 1 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 21.3 | Watson | 6 | 1 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 22.1 | Watson | 6 | 1 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 22.2 | Watson | 6 | 1 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 22.3 | Watson | 6 | 1 | 90 cm | 30 | 3.2, 6.4, 1.6 | 2.4, 5.6, 1.6 | US Plastic |
| | Marlow | | | length of | | (STHT-C- | (STHT-C- | ⅜-⅜-⅜″ |
| | Flexicon | | | ⅜″ ID Si | | 125-2) | 093-2) | tee |
| | (PD12I) | | | tubing in | | | | (62210) |
| | | | | loop | | | | |
| | | | | (STHT-C- | | | | |
| | | | | 375-4 | | | | |
| | | | | [tubing]) | | | | |
| 24.1 | Chemyx | None | None | None | 30 | 3.2, 6.4, 1.6 | 2.4, 5.6, 1.6 | N/A |
| | Dual | | | | | (STHT-C- | (STHT-C- | |
| | Syringe | | | | | 125-2) | 093-2) | |
| | Pump | | | | | | | |
| | (Fusion | | | | | | | |
| | 200) | | | | | | | |
| 24.2 | Chemyx | None | None | None | 30 | 3.2, 6.4, 1.6 | 2.4, 5.6, 1.6 | N/A |
| | Dual | | | | | (STHT-C- | (STHT-C- | |
| | Syringe | | | | | 125-2) | 093-2) | |
| | Pump | | | | | | | |
| | (Fusion | | | | | | | |
| | 200) | | | | | | | |
| 24.3 | Chemyx | None | None | None | 30 | 3.2, 6.4, 1.6 | 2.4, 5.6, 1.6 | N/A |
| | Dual | | | | | (STHT-C- | (STHT-C- | |
| | Syringe | | | | | 125-2) | 093-2) | |
| | Pump | | | | | | | |
| | (Fusion | | | | | | | |
| | 200) | | | | | | | |
| 25.1 | Chemyx | None | None | None | 30 | 3.2, 6.4, 1.6 | 2.4, 5.6, 1.6 | N/A |
| | Dual | | | | | (STHT-C- | (STHT-C- | |
| | Syringe | | | | | 125-2) | 093-2) | |
| | Pump | | | | | | | |
| | (Fusion | | | | | | | |
| | 200) | | | | | | | |
| 25.2 | Chemyx | None | None | None | 30 | 3.2, 6.4, 1.6 | 2.4, 5.6, 1.6 | N/A |
| | Dual | | | | | (STHT-C- | (STHT-C- | |
| | Syringe | | | | | 125-2) | 093-2) | |
| | Pump | | | | | | | |
| | (Fusion | | | | | | | |
| | 200) | | | | | | | |
| 25.3 | Chemyx | None | None | None | 30 | 3.2, 6.4, 1.6 | 2.4, 5.6, 1.6 | N/A |
| | Dual | | | | | (STHT-C- | (STHT-C- | |
| | Syringe | | | | | 125-2) | 093-2) | |
| | Pump | | | | | | | |
| | (Fusion | | | | | | | |
| | 200) | | | | | | | |
| 26.1 | Syringe | None | None | None | 30 | 3.2, 6.4, 1.6 | 2.4, 5.6, 1.6 | N/A |
| | Pump | | | | | (STHT-C- | (STHT-C- | |
| | (KD | | | | | 125-2) | 093-2) | |
| | Scientific | | | | | | | |
| | Legato | | | | | | | |
| | 200) | | | | | | | |
| 26.2 | Syringe | None | None | None | 30 | 3.2, 6.4, 1.6 | 2.4, 5.6, 1.6 | N/A |
| | Pump | | | | | (STHT-C- | (STHT-C- | |
| | (KD | | | | | 125-2) | 093-2) | |
| | Scientific | | | | | | | |
| | Legato | | | | | | | |
| | 200) | | | | | | | |
| 26.3 | Syringe | None | None | None | 30 | 3.2, 6.4, 1.6 | 2.4, 5.6, 1.6 | N/A |
| | Pump | | | | | (STHT-C- | (STHT-C- | |
| | (KD | | | | | 125-2) | 093-2) | |
| | Scientific | | | | | | | |
| | Legato | | | | | | | |
| | 200) | | | | | | | |

TABLE 5B

| | Average | Maximum | Minimum | Maximum | Minimum | | Relative | |
|---|---|---|---|---|---|---|---|---|
| | flow | flow | flow | flow | flow | | standard | |
| Ex. | rate | rate | rate | rate | rate | Standard | deviation | Level of |
| No. | (mL/min) | (mL/min) | (mL/min) | (%) | (%) | deviation | (%) | pulsation |
| 19.1 | 82.37 | 84.36 | 78.55 | 2.41 | 4.64 | 1 | 2 | 7 |
| 19.2 | 79.34 | 82.03 | 76.69 | 3.4 | 3.34 | 1 | 1 | 7 |
| 19.3 | 161.28 | 165.66 | 153.35 | 2.72 | 4.91 | 3 | 2 | 8 |
| 20.1 | 80.65 | 114.27 | 49.74 | 41.68 | 38.32 | 21 | 27 | 80 |
| 20.2 | 77.6 | 109.98 | 42.41 | 41.73 | 45.34 | 22 | 28 | 87 |
| 20.3 | 156.51 | 216.66 | 91.88 | 38.43 | 41.29 | 41 | 26 | 80 |
| 21.1 | 41.66 | 44.91 | 38.3 | 7.81 | 8.06 | 1 | 3 | 16 |
| 21.2 | 41.83 | 44.89 | 38.96 | 7.31 | 6.85 | 1 | 3 | 14 |
| 21.3 | 91.27 | 96.43 | 83.32 | 5.65 | 8.71 | 3 | 3 | 14 |
| 22.1 | 70.77 | 74.47 | 67.28 | 5.23 | 4.93 | 2 | 2 | 10 |
| 22.2 | 74.40 | 77.72 | 71.86 | 4.47 | 3.42 | 1 | 2 | 8 |
| 22.3 | 160.92 | 167.84 | 153.35 | 4.3 | 4.7 | 3 | 2 | 9 |
| 24.1 | 50.37 | 56.01 | 43.13 | 11.20 | 14.37 | 3 | 6 | 26 |
| 24.2 | 51.79 | 58.74 | 45.69 | 13.43 | 11.77 | 3 | 6 | 25 |
| 24.3 | 111.29 | 124.08 | 96.66 | 11.50 | 13.15 | 7 | 6 | 25 |
| 25.1 | 26.31 | 29.50 | 22.14 | 12.14 | 15.85 | 2 | 7 | 28 |
| 25.2 | 25.99 | 29.47 | 22.57 | 13.39 | 13.16 | 1 | 5 | 27 |
| 25.3 | 56.57 | 60.83 | 48.87 | 7.54 | 13.60 | 3 | 6 | 21 |
| 26.1 | 66.08 | 71.68 | 59.49 | 8.47 | 9.98 | 2 | 4 | 18 |
| 26.2 | 71.84 | 75.50 | 67.62 | 5.09 | 5.87 | 2 | 3 | 11 |
| 26.3 | 140.65 | 146.53 | 132.67 | 4.18 | 5.68 | 3 | 2 | 10 |

The results of Experiments 19.1, 20.1, 21.1, and 22.1 are all from the flow meter attached to the first source inlet. The results of Experiments 19.2, 20.2, 21.2, and 22.2 are all from the flow meter attached to the second source inlet. The results of Experiments 19.3, 20.3, 21.3, and 22.3 are all from the flow meter after the Y-connector or mixer of the combined first and second source. As reported in Table 5B, the flow rates, measured after both inlets were mixed, were approximately 91 mL/min and 161 mL/min and their corresponding LoP were around 15 (14-16) and around 9 (8-10) for Experiments 21 and 22, respectively. Although the LoP at the higher flow rate (Experiment 22) was 10 or less, a setup with a single pump head may not be robust across a wide variety of flow rates since the LoP of Experiment 21 increased.

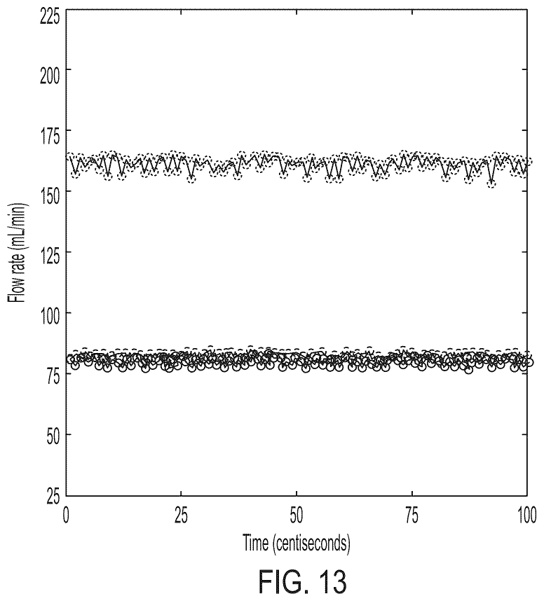

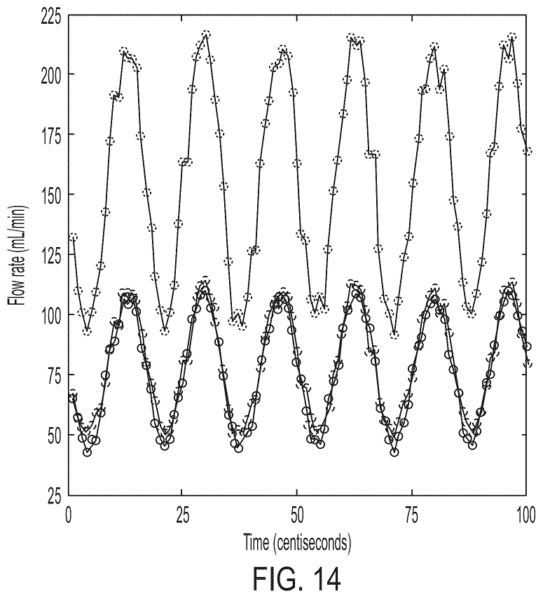

As reported in Table 5B, the LoP of each inlet for Experiment 20 (no dampening) was 80 and 87, and the LoP of the outlet was 80. In contrast, the LoPs of Experiment 19 (dampening) using the loop dampener were both 7 and the LoP at the outlet was 8. These results meet the acceptable goal for producing, mixing, transferring and/or manufacturing the pharmaceutical compositions and formulations described herein, including, e.g., pharmaceutical compositions comprising RNA or lipids, such as an RNA vaccine, of achieving an LoP of less than 10 (e.g., to adequately control flow rates of multiple pumps and/or fluid sources to ensure appropriate mixing of pharmaceutical compositions, and to achieve equivalent or better LoP values than generally achieved with syringe pumps), and the dampening loop meets all of the GMP process goals outlined earlier, while being simple and cost-effective to implement and compatible with single use sourcing. FIG. 13 illustrates the flowrate of water through the peristaltic pump system in accordance with Experiment 19 and FIG. 14 illustrates the flowrate of water through the peristaltic pump in accordance with the experiment of Experiment 20.

The results of Experiments 24.1, 25.1, and 26.1 are all from the flow meter attached to the first source inlet. The results of Experiments 14.2, 25.2, and 26.2 are all from the flow meter attached to the second source inlet. The results of Experiments 24.3, 25.3, 26.3 are all from the flow meter after the Y-connector or mixer of the combined first and second source.

As reported in Table 5B, the flow rates, measured after both inlets were mixed, were approximately 111 mL/min and 57 mL/min and their corresponding LoP were around an average of 25.5 (25-26) and around 24.5 (21-28) for Experiments 24 and 25, respectively. These experiments used an off-the-shelf syringe pump that was not specially designed to reduce pump pulsations but demonstrate that standard units do not meet the acceptable goal with respect to achieving a LoP value of less than 10. Experiment 26 also used an off-the-shelf syringe pump. The pulsation in commercially available syringe pumps may vary but this type of system is not pulsation-free. As reported in Table 5B, the flow rate, measured after both inlets were mixed, was approximately 141 mL/min and the corresponding LoP was an average around 14 (10-18). These results confirmed that Experiment 19 (loop dampener) achieved better LoP values than both syringe pump systems.

FIG. 15 describes a peristaltic pump, dampener and tubing kit system capable of achieving an LoP of less than 10 from two fluid sources, including, e.g., the pharmaceutical compositions described herein, and in particular, the pharmaceutical compositions comprising a pharmaceutical composition comprising RNA, RNA molecules or RNA vaccine, and another pharmaceutical composition comprising one or more lipids, which can be mixed to create, transfer or manufacture a final pharmaceutical composition comprising RNA-lipoplexes or RNA liposomes.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The present invention is not intended to be limited in scope to the particular disclosed embodiments, which are provided, for example, to illustrate various aspects of the invention. Various modifications to the device and methods described will become apparent from the description and teachings herein. Such variations may be practiced without departing from the true scope and spirit of the disclosure and are intended to fall within the scope of the present disclosure.

### INFORMAL SEQUENCE LISTING

All polynucleotide sequences are depicted in the 5′→3 direction. All polypeptide sequences are depicted in the N-terminal to C-terminal direction.

Full PCV RNA 5′ constant sequence
(SEQ ID NO: 1)
GGCGAACUAGUAUUCUUCUGGUCCCCACAGACUCAGAGAGAACCCGCC
ACCAUGAGAGUGAUGGCCCCCAGAACCCUGAUCCUGCUGCUGUCUGGC
GCCCUGGCCCUGACAGAGACAUGGGCCGGAAGC
Full PCV RNA 3′ constant sequence
(SEQ ID NO: 2)
AUCGUGGGAAUUGUGGCAGGACUGGCAGUGCUGGCCGUGGUGGUGAUC
GGAGCCGUGGUGGCUACCGUGAUGUGCAGACGGAAGUCCAGCGGAGGC
AAGGGCGGCAGCUACAGCCAGGCCGCCAGCUCUGAUAGCGCCCAGGGC
AGCGACGUGUCACUGACAGCCUAGUAACUCGAGCUGGUACUGCAUGCA
CGCAAUGCUAGCUGCCCCUUUCCCGUCCUGGGUACCCCGAGUCUCCCC
CGACCUCGGGUCCCAGGUAUGCUCCCACCUCCACCUGCCCCACUCACC
ACCUCUGCUAGUUCCAGACACCUCCCAAGCACGCAGCAAUGCAGCUCA
AAACGCUUAGCCUAGCCACACCCCCACGGGAAACAGCAGUGAUUAACC
UUUAGCAAUAAACGAAAGUUUAACUAAGCUAUACUAACCCCAGGGUUG
GUCAAUUUCGUGCCAGCCACACCGAGACCUGGUCCAGAGUCGCUAGCC
GCGUCGCU
Full PCV Kozak RNA
(SEQ ID NO: 3)
GGCGAACUAGUAUUCUUCUGGUCCCCACAGACUCAGAGAGAACCCGCC
ACC
Full PCV Kozak DNA
(SEQ ID NO: 4)
GGCGAACTAGTATTCTTCTGGTCCCCACAGACTCAGAGAGAACCCGCC
ACC
short Kozak RNA
(SEQ ID NO: 5)
UUCUUCUGGUCCCCACAGACUCAGAGAGAACCCGCCACC
short Kozak DNA
(SEQ ID NO: 6)
TTCTTCTGGTCCCCACAGACTCAGAGAGAACCCGCCACC
sec RNA
(SEQ ID NO: 7)
AUGAGAGUGAUGGCCCCCAGAACCCUGAUCCUGCUGCUGUCUGGCGCC
CUGGCCCUGACAGAGACAUGGGCCGGAAGC
sec DNA
(SEQ ID NO: 8)
ATGAGAGTGATGGCCCCCAGAACCCTGATCCTGCTGCTGTCTGGCGCC
CTGGCCCTGACAGAGACATGGGCCGGAAGC
sec protein
(SEQ ID NO: 9)
MRVMAPRTLILLLSGALALTETWAGS
MITD RNA
(SEQ ID NO: 10)
AUCGUGGGAAUUGUGGCAGGACUGGCAGUGCUGGCCGUGGUGGUGAUC
GGAGCCGUGGUGGCUACCGUGAUGUGCAGACGGAAGUCCAGCGGAGGC
AAGGGCGGCAGCUACAGCCAGGCCGCCAGCUCUGAUAGCGCCCAGGGC
AGCGACGUGUCACUGACAGCC
MITD DNA
(SEQ ID NO: 11)
ATCGTGGGAATTGTGGCAGGACTGGCAGTGCTGGCCGTGGTGGTGATC
GGAGCCGTGGTGGCTACCGTGATGTGCAGACGGAAGTCCAGCGGAGGC
AAGGGCGGCAGCTACAGCCAGGCCGCCAGCTCTGATAGCGCCCAGGGC
AGCGACGTGTCACTGACAGCC
MITD protein
(SEQ ID NO: 12)
IVGIVAGLAVLAVVVIGAVVATVMCRRKSSGGKGGSYSQAASSDSAQG
SDVSLTA
Full PCV FI RNA
(SEQ ID NO: 13)
CUCGAGCUGGUACUGCAUGCACGCAAUGCUAGCUGCCCCUUUCCCGUC
CUGGGUACCCCGAGUCUCCCCCGACCUCGGGUCCCAGGUAUGCUCCCA
CCUCCACCUGCCCCACUCACCACCUCUGCUAGUUCCAGACACCUCCCA
AGCACGCAGCAAUGCAGCUCAAAACGCUUAGCCUAGCCACACCCCCAC
GGGAAACAGCAGUGAUUAACCUUUAGCAAUAAACGAAAGUUUAACUAA
GCUAUACUAACCCCAGGGUUGGUCAAUUUCGUGCCAGCCACACCGAGA
CCUGGUCCAGAGUCGCUAGCCGCGUCGCU
Full PCV FI DNA
(SEQ ID NO: 14)
CTGGTACTGCATGCACGCAATGCTAGCTGCCCCTTTCCCGTCCTGGGT
ACCCCGAGTCTCCCCCGACCTCGGGTCCCAGGTATGCTCCCACCTCCA
CCTGCCCCACTCACCACCTCTGCTAGTTCCAGACACCTCCCAAGCACG
CAGCAATGCAGCTCAAAACGCTTAGCCTAGCCACACCCCCACGGGAAA
CAGCAGTGATTAACCTTTAGCAATAAACGAAAGTTTAACTAAGCTATA
CTAACCCCAGGGTTGGTCAATTTCGTGCCAGCCACACCGAGACCTGGT
CCAGAGTCGCTAGCCGCGTCGCT
F element RNA
(SEQ ID NO: 15)
CUGGUACUGCAUGCACGCAAUGCUAGCUGCCCCUUUCCCGUCCUGGGU
ACCCCGAGUCUCCCCCGACCUCGGGUCCCAGGUAUGCUCCCACCUCCA
CCUGCCCCACUCACCACCUCUGCUAGUUCCAGACACCUCC
F element DNA
(SEQ ID NO: 16)
CTGGTACTGCATGCACGCAATGCTAGCTGCCCCTTTCCCGTCCTGGGT
ACCCCGAGTCTCCCCCGACCTCGGGTCCCAGGTATGCTCCCACCTCCA
CCTGCCCCACTCACCACCTCTGCTAGTTCCAGACACCTCC
I element RNA
(SEQ ID NO: 17)
CAAGCACGCAGCAAUGCAGCUCAAAACGCUUAGCCUAGCCACACCCCC
ACGGGAAACAGCAGUGAUUAACCUUUAGCAAUAAACGAAAGUUUAACU
AAGCUAUACUAACCCCAGGGUUGGUCAAUUUCGUGCCAGCCACACCG
I element DNA
(SEQ ID NO: 18)
CAAGCACGCAGCAATGCAGCTCAAAACGCTTAGCCTAGCCACACCCCC
ACGGGAAACAGCAGTGATTAACCTTTAGCAATAAACGAAAGTTTAACT
AAGCTATACTAACCCCAGGGTTGGTCAATTTCGTGCCAGCCACACCG
linker RNA
(SEQ ID NO: 19)
GGCGGCUCUGGAGGAGGCGGCUCCGGAGGC
linker DNA
(SEQ ID NO: 20)
GGCGGCTCTGGAGGAGGCGGCTCCGGAGGC
linker protein
(SEQ ID NO: 21)
GGSGGGGSGG
Full PCV DNA 5′ constant sequence
(SEQ ID NO: 22)
GGCGAACTAGTATTCTTCTGGTCCCCACAGACTCAGAGAGAACCCGCC
ACCATGAGAGTGATGGCCCCCAGAACCCTGATCCTGCTGCTGTCTGGC
GCCCTGGCCCTGACAGAGACATGGGCCGGAAGC
Full PCV DNA 3′ constant sequence
(SEQ ID NO: 23)
ATCGTGGGAATTGTGGCAGGACTGGCAGTGCTGGCCGTGGTGGTGATC
GGAGCCGTGGTGGCTACCGTGATGTGCAGACGGAAGTCCAGCGGAGGC
AAGGGCGGCAGCTACAGCCAGGCCGCCAGCTCTGATAGCGCCCAGGGC
AGCGACGTGTCACTGACAGCCTAGTAACTCGAGCTGGTACTGCATGCA
CGCAATGCTAGCTGCCCCTTTCCCGTCCTGGGTACCCCGAGTCTCCCC
CGACCTCGGGTCCCAGGTATGCTCCCACCTCCACCTGCCCCACTCACC
ACCTCTGCTAGTTCCAGACACCTCCCAAGCACGCAGCAATGCAGCTCA
AAACGCTTAGCCTAGCCACACCCCCACGGGAAACAGCAGTGATTAACC
TTTAGCAATAAACGAAAGTTTAACTAAGCTATACTAACCCCAGGGTTG
GTCAATTTCGTGCCAGCCACACCGAGACCTGGTCCAGAGTCGCTAGCC
GCGTCGCT
Full PCV RNA with 5′ GG from cap
(SEQ ID NO: 24)
GGGGCGAACU AGUAUUCUUC UGGUCCCCAC AGACUCAGAG
AGAACCCGCC ACCAUGAGAG UGAUGGCCCC CAGAACCCUG
AUCCUGCUGC UGUCUGGCGC CCUGGCCCUG ACAGAGACAU
GGGCCGGAAG CNAUCGUGGGA AUUGUGGCAG GACUGGCAGU
GCUGGCCGUG GUGGUGAUCG GAGCCGUGGU GGCUACCGUG
AUGUGCAGAC GGAAGUCCAG CGGAGGCAAG GGCGGCAGCU
ACAGCCAGGC CGCCAGCUCU GAUAGCGCCC AGGGCAGCGA
CGUGUCACUG ACAGCCUAGU AACUCGAGCU GGUACUGCAU
GCACGCAAUG CUAGCUGCCC CUUUCCCGUC CUGGGUACCC
CGAGUCUCCC CCGACCUCGG GUCCCAGGUA UGCUCCCACC
UCCACCUGCC CCACUCACCA CCUCUGCUAG UUCCAGACAC
CUCCCAAGCA CGCAGCAAUG CAGCUCAAAA CGCUUAGCCU
AGCCACACCC CCACGGGAAA CAGCAGUGAU UAACCUUUAG
CAAUAAACGA AAGUUUAACU AAGCUAUACU AACCCCAGGG
UUGGUCAAUU UCGUGCCAGC CACACCGAGA CCUGGUCCAG
AGUCGCUAGC CGCGUCGCUA AAAAAAAAAA AAAAAAAAAA
AAAAAAAAAA AAAAAAAAAA AAAAAAAAAA AAAAAAAAAA
AAAAAAAAAA AAAAAAAAAA AAAAAAAAAA AAAAAAAAAA
AAAAAAAAAA AAAAAAAAA

1. A tubing kit for forming a mixture comprising:
a first portion of tubing configured to be fluidly connected to a container containing a first composition;
a second portion of tubing configured to be fluidly connected to a container containing a second composition;
a dampener fluidly connected to the first portion of tubing and fluidly connected to the second portion of tubing a mixer for mixing the first composition from the first portion of tubing and the second composition from the second portion of tubing;
a mixture container for collecting the mixed first composition and second composition from the mixer,
wherein the first portion of tubing is configured to be connected to at least one peristaltic pump head for pumping the first composition from the container containing the first composition to the mixture container, and the second portion of tubing is configured to be connected to at least one peristaltic pump head for pumping the second composition from the container containing the second composition to the mixture container.

2. The tubing kit of claim 1, wherein the dampener comprises an enclosed volume of fluid.

3. The tubing kit of claim 2, wherein the fluid is air.

4. The tubing kit of claim 1, wherein the dampener is a tubing dampener.

5. The tubing kit of claim 1, wherein the dampener comprises a flexible membrane.

6. The tubing kit of claim 1, further comprising a first tee connector that fluidly connects the dampener, the first portion of tubing, and a first mixer input portion of tubing, wherein the first mixer input portion of tubing fluidly connects to the mixer.

7. The tubing kit of claim 6, further comprising a second tee connector that fluidly connects the dampener, the second portion of tubing, and a second mixer input portion of tubing, wherein the second mixer input portion of tubing fluidly connects to the mixer.

8. The tubing kit of claim 1, wherein the first portion of tubing comprises a first segment of tubing and a second segment of tubing, wherein the first segment of tubing and the second segment of tubing are fluidly connected in parallel.

9. The tubing kit of claim 8, wherein the first segment of tubing is configured to be connected to a first peristaltic pump head, and the second segment of tubing is configured to be connected to a second peristaltic pump head.

10. The tubing kit of claim 1, wherein the second portion of tubing comprises a third segment of tubing and a fourth segment of tubing, wherein the third portion of tubing and the fourth portion of tubing are fluidly connected in parallel.

11. The tubing kit of claim 10, wherein the third segment of tubing is configured to be connected to a third peristaltic pump head, and the fourth segment of tubing is configured to be connected to a fourth peristaltic pump head.

12. The tubing kit of claim 1, wherein the mixer comprises an input fluidly connected to the first portion of tubing, an input fluidly connected to the second portion of tubing, and output fluidly connected to the mixture container.

13. The tubing kit of claim 1, wherein the mixer comprises a Y-connector, a helical mixer, or a static mixer.

14. The tubing kit of claim 1, further comprising a first dampener connector that fluidly connects the first portion of tubing to the dampener and to the mixer and a second dampener connector that fluidly connects the second portion of the tubing to the dampener and to the mixer.

15. The tubing kit of claim 1, wherein the mixture container is a bag, vessel, or bottle.

16. A system for forming a pharmaceutical composition or mixture of pharmaceutical compositions, the system comprising:
a first container containing a first pharmaceutical composition;
a second container containing a second pharmaceutical composition;
a first portion of tubing fluidly connected to the first container;
a second portion of tubing fluidly connected to the second container;
a dampener fluidly connected to the first portion of tubing and fluidly connected to the second portion of tubing;
a mixer for mixing the first pharmaceutical composition from the first portion of tubing and the second pharmaceutical composition from the second portion of tubing; and
a mixture container for collecting the mixed first pharmaceutical composition and second pharmaceutical composition from the mixer.

17. The system of claim 16, further comprising at least one peristaltic pump head connected to the first portion of tubing for pumping the first composition from the container containing the first composition to the mixture container, and at least one peristaltic pump connected to the second portion of tubing for pumping the second composition from the container containing the first composition to the mixture container.

18. (canceled)

19. (canceled)

20. (canceled)

21. (canceled)

22. (Canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. A method for transferring pharmaceutical compositions using peristaltic pumps comprising:
pumping a first composition from a first container through a first portion of tubing using at least one peristaltic pump;
pumping a second composition from a second container through a second portion of tubing using at least one peristaltic pump; and
dampening pulse in a fluid flow of the first composition in the first portion of tubing and dampening pulses in a fluid flow of the second composition in the second portion of tubing using a dampener fluid connected to the first portion of tubing and fluidly connected to the second portion of tubing.

49. The method of claim 48, further comprising mixing the first composition from the first portion of tubing and the second composition from the second portion of tubing in a mixer fluidly connected to the first portion of tubing and the second portion of tubing. (Original) The method of claim 49, further comprising depositing the mixture containing the first composition and the second composition into a mixture container that is fluidly connected to the mixture.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)
